SEQUENCE LISTING (152 sequences)

SEQ ID NO: 1 - DNA, 29848 bp, unidentified
  source 1..29848: mol_type = other DNA
aaaggtttataccttcccaggtaacaaaccaaccaactttcgatctcttgtagatctgttctctaaacgaactttaaaatctgtgtggctgtcactcggctgcatgcttagtgcactcacgcagtataattaataactaattactgtcgttgacaggacacgagtaactcgtctatcttctgcaggctgcttacggtttcgtccgtgttgcagccgatcatcagcacatctaggtttcgtccgggtgtgaccgaaaggtaagatggagagccttgtccctggtttcaacgagaaaacacacgtccaactcagtttgcctgttttacaggttcgcgacgtgctcgtacgtggcttttcagaggcacgtcaacatcttaaagatggcacttgtggcttagtagaagttgaaaaaggcgttttgcctcaacttgaacagccctatgtgttcatcaaacgttcggatgctcgaactgcacctcatggtcatgttatggttgagctggtagcagaactcgaaggcattcagtacggtcgtagtggtgagacacttggtgtccttgtccctcatgtgggcgaaataccagtggcttaccgcaaggttcttcttcgtaagaacggtaataaaggagctggtggccatagttacggcgccgatctaaagtcatttgacttaggcgacgagcttggcactgatccttatgaagattttcaagaaaactggaacactaaacatagcagtggtgttacccgtgaactcatgcgtgagcttaacggaggggcatacactcgctatgtcgataacaacttctgtggccctgatggctaccctcttgagtgcattaaagaccttctagcacgtgctggtaaagcttcatgcactttgtccgaacaactggactttattgacactaagaggggtgtatactgctgccgtgaacatgagcatgaaattgcttggtacacggaacgttctgaaaagagctatgaattgcagacaccttttgaaattaaattggcaaagaaatttgacaccttcaatggggaatgtccaaattttgtatttcccttaaattccataatcaagactattcaaccaagggttgaaaagaaaaagcttgatggctttatgggtagaattcgatctgtctatccagttgcgtcaccaaatgaatgcaaccaaatgtgcctttcaactctcatgaagtgtgatcattgtggtgaaacttcatggcagacgggcgattttgttaaagccacttgcgaattttgtggcactgagaatttgactaaagaaggtgccactacttgtggttacttaccccaaaatgctgttgttaaaatttattgtccagcatgtcacaattcagaagtaggacctgagcatagtcttgccgaataccataatgaatctggcttgaaaaccattcttcgtaagggtggtcgcactattgcctttggaggctgtgtgttctcttatgttggttgccataacaagtgtgcctattgggttccacgtgctagcgctaacataggttgtaaccatacaggtgttgttggagaaggttccgaaggtcttaatgacaaccttcttgaaatactccaaaaagagaaagtcaacatcaatattgttggtgactttaaacttaatgaagagatcgccattattttggcatctttttctgcttccacaagtgcttttgtggaaactgtgaaaggtttggattataaagcattcaaacaaattgttgaatcctgtggtaattttaaagttacaaaaggaaaagctaaaaaaggtgcctggaatattggtgaacagaaatcaatactgagtcctctttatgcatttgcatcagaggctgctcgtgttgtacgatcaattttctctcgcactcttgaaactgctcaaaattctgtgcgtgttttacagaaggccgctataacaatactagatggaatttcacagtattcactgagactcattgatgctatgatgttcacatctgatttggctactaacaatctagttgtaatggcctacattacaggtggtgttgttcagttgacttcgcagtggctaactaacatctttggcactgtttatgaaaaactcaaacccgtccttgattggcttgaagagaagtttaaggaaggtgtagagtttcttagagacggttgggaaattgttaaatttatctcaacctgtgcttgtgaaattgtcggtggacaaattgtcacctgtgcaaaggaaattaaggagagtgttcagacattctttaagcttgtaaataaatttttggctttgtgtgctgactctatcattattggtggagctaaacttaaagccttgaatttaggtgaaacatttgtcacgcactcaaagggattgtacagaaagtgtgttaaatccagagaagaaactggcctactcatgcctctaaaagccccaaaagaaattatcttcttagagggagaaacacttcccacagaagtgttaacagaggaagttgtcttgaaaactggtgatttacaaccattagaacaacctactagtgaagctgttgaagctccattggttggtacaccagtttgtattaacgggcttatgttgctcgaaatcaaagacacagaaaagtactgtgcccttgcacctaatatgatggtaacaaacaataccttcacactcaaaggcggtgcaccaacaaaggttacttttggtgatgacactgtgatagaagtgcaaggttacaagagtgtgaatatcacttttgaacttgatgaaaggattgataaagtacttaatgagaagtgctctgcctatacagttgaactcggtacagaagtaaatgagttcgcctgtgttgtggcagatgctgtcataaaaactttgcaaccagtatctgaattacttacaccactgggcattgatttagatgagtggagtatggctacatactacttatttgatgagtctggtgagtttaaattggcttcacatatgtattgttctttctaccctccagatgaggatgaagaagaaggtgattgtgaagaagaagagtttgagccatcaactcaatatgagtatggtactgaagatgattaccaaggtaaacctttggaatttggtgccacttctgctgctcttcaacctgaagaagagcaagaagaagattggttagatgatgatagtcaacaaactgttggtcaacaagacggcagtgaggacaatcagacaactactattcaaacaattgttgaggttcaacctcaattagagatggaacttacaccagttgttcagactattgaagtgaatagttttagtggttatttaaaacttactgacaatgtatacattaaaaatgcagacattgtggaagaagctaaaaaggtaaaaccaacagtggttgttaatgcagccaatgtttaccttaaacatggaggaggtgttgcaggagccttaaataaggctactaacaatgccatgcaagttgaatctgatgattacatagctactaatggaccacttaaagtgggtggtagttgtgttttaagcggacacaatcttgctaaacactgtcttcatgttgtcggcccaaatgttaacaaaggtgaagacattcaacttcttaagagtgcttatgaaaattttaatcagcacgaagttctacttgcaccattattatcagctggtatttttggtgctgaccctatacattctttaagagtttgtgtagatactgttcgcacaaatgtctacttagctgtctttgataaaaatctctatgacaaacttgtttcaagctttttggaaatgaagagtgaaaagcaagttgaacaaaagatcgctgagattcctaaagaggaagttaagccatttataactgaaagtaaaccttcagttgaacagagaaaacaagatgataagaaaatcaaagcttgtgttgaagaagttacaacaactctggaagaaactaagttcctcacagaaaacttgttactttatattgacattaatggcaatcttcatccagattctgccactcttgttagtgacattgacatcactttcttaaagaaagatgctccatatatagtgggtgatgttgttcaagagggtgttttaactgctgtggttatacctactaaaaaggctggtggcactactgaaatgctagcgaaagctttgagaaaagtgccaacagacaattatataaccacttacccgggtcagggtttaaatggttacactgtagaggaggcaaagacagtgcttaaaaagtgtaaaagtgccttttacattctaccatctattatctctaatgagaagcaagaaattcttggaactgtttcttggaatttgcgagaaatgcttgcacatgcagaagaaacacgcaaattaatgcctgtctgtgtggaaactaaagccatagtttcaactatacagcgtaaatataagggtattaaaatacaagagggtgtggttgattatggtgctagattttacttttacaccagtaaaacaactgtagcgtcacttatcaacacacttaacgatctaaatgaaactcttgttacaatgccacttggctatgtaacacatggcttaaatttggaagaagctgctcggtatatgagatctctcaaagtgccagctacagtttctgtttcttcacctgatgctgttacagcgtataatggttatcttacttcttcttctaaaacacctgaagaacattttattgaaaccatctcacttgctggttcctataaagattggtcctattctggacaatctacacaactaggtatagaatttcttaagagaggtgataaaagtgtatattacactagtaatcctaccacattccacctagatggtgaagttatcacctttgacaatcttaagacacttctttctttgagagaagtgaggactattaaggtgtttacaacagtagacaacattaacctccacacgcaagttgtggacatgtcaatgacatatggacaacagtttggtccaacttatttggatggagctgatgttactaaaataaaacctcataattcacatgaaggtaaaacattttatgttttacctaatgatgacactctacgtgttgaggcttttgagtactaccacacaactgatcctagttttctgggtaggtacatgtcagcattaaatcacactaaaaagtggaaatacccacaagttaatggtttaacttctattaaatgggcagataacaactgttatcttgccactgcattgttaacactccaacaaatagagttgaagtttaatccacctgctctacaagatgcttattacagagcaagggctggtgaagctgctaacttttgtgcacttatcttagcctactgtaataagacagtaggtgagttaggtgatgttagagaaacaatgagttacttgtttcaacatgccaatttagattcttgcaaaagagtcttgaacgtggtgtgtaaaacttgtggacaacagcagacaacccttaagggtgtagaagctgttatgtacatgggcacactttcttatgaacaatttaagaaaggtgttcagataccttgtacgtgtggtaaacaagctacaaaatatctagtacaacaggagtcaccttttgttatgatgtcagcaccacctgctcagtatgaacttaagcatggtacatttacttgtgctagtgagtacactggtaattaccagtgtggtcactataaacatataacttctaaagaaactttgtattgcatagacggtgctttacttacaaagtcctcagaatacaaaggtcctattacggatgttttctacaaagaaaacagttacacaacaaccataaaaccagttacttataaattggatggtgttgtttgtacagaaattgaccctaagttggacaattattataagaaagacaattcttatttcacagagcaaccaattgatcttgtaccaaaccaaccatatccaaacgcaagcttcgataattttaagtttgtatgtgataatatcaaatttgctgatgatttaaaccagttaactggttataagaaacctgcttcaagagagcttaaagttacatttttccctgacttaaatggtgatgtggtggctattgattataaacactacacaccctcttttaagaaaggagctaaattgttacataaacctattgtttggcatgttaacaatgcaactaataaagccacgtataaaccaaatacctggtgtatacgttgtctttggagcacaaaaccagttgaaacatcaaattcgtttgatgtactgaagtcagaggacgcgcagggaatggataatcttgcctgcgaagatctaaaaccagtctctgaagaagtagtggaaaatcctaccatacagaaagacgttcttgagtgtaatgtgaaaactaccgaagttgtaggagacattatacttaaaccagcaaataatagtttaaaaattacagaagaggttggccacacagatctaatggctgcttatgtagacaattctagtcttactattaagaaacctaatgaattatctagagtattaggtttgaaaacccttgctactcatggtttagctgctgttaatagtgtcccttgggatactatagctaattatgctaagccttttcttaacaaagttgttagtacaactactaacatagttacacggtgtttaaaccgtgtttgtactaattatatgccttatttctttactttattgctacaattgtgtacttttactagaagtacaaattctagaattaaagcatctatgccgactactatagcaaagaatactgttaagagtgtcggtaaattttgtctagaggcttcatttaattatttgaagtcacctaatttttctaaactgataaatattataatttggtttttactattaagtgtttgcctaggttctttaatctactcaaccgctgctttaggtgttttaatgtctaatttaggcatgccttcttactgtactggttacagagaaggctatttgaactctactaatgtcactattgcaacctactgtactggttctataccttgtagtgtttgtcttagtggtttagattctttagacacctatccttctttagaaactatacaaattaccatttcatcttttaaatgggatttaactgcttttggcttagttgcagagtggtttttggcatatattcttttcactaggtttttctatgtacttggattggctgcaatcatgcaattgtttttcagctattttgcagtacattttattagtaattcttggcttatgtggttaataattaatcttgtacaaatggccccgatttcagctatggttagaatgtacatcttctttgcatcattttattatgtatggaaaagttatgtgcatgttgtagacggttgtaattcatcaacttgtatgatgtgttacaaacgtaatagagcaacaagagtcgaatgtacaactattgttaatggtgttagaaggtccttttatgtctatgctaatggaggtaaaggcttttgcaaactacacaattggaattgtgttaattgtgatacattctgtgctggtagtacatttattagtgatgaagttgcgagagacttgtcactacagtttaaaagaccaataaatcctactgaccagtcttcttacatcgttgatagtgttacagtgaagaatggttccatccatctttactttgataaagctggtcaaaagacttatgaaagacattctctctctcattttgttaacttagacaacctgagagctaataacactaaaggttcattgcctattaatgttatagtttttgatggtaaatcaaaatgtgaagaatcatctgcaaaatcagcgtctgtttactacagtcagcttatgtgtcaacctatactgttactagatcaggcattagtgtctgatgttggtgatagtgcggaagttgcagttaaaatgtttgatgcttacgttaatacgttttcatcaacttttaacgtaccaatggaaaaactcaaaacactagttgcaactgcagaagctgaacttgcaaagaatgtgtccttagacaatgtcttatctacttttatttcagcagctcggcaagggtttgttgattcagatgtagaaactaaagatgttgttgaatgtcttaaattgtcacatcaatctgacatagaagttactggcgatagttgtaataactatatgctcacctataacaaagttgaaaacatgacaccccgtgaccttggtgcttgtattgactgtagtgcgcgtcatattaatgcgcaggtagcaaaaagtcacaacattgctttgatatggaacgttaaagatttcatgtcattgtctgaacaactacgaaaacaaatacgtagtgctgctaaaaagaataacttaccttttaagttgacatgtgcaactactagacaagttgttaatgttgtaacaacaaagatagcacttaagggtggtaaaattgttaataattggttgaagcagttaattaaagttacacttgtgttcctttttgttgctgctattttctatttaataacacctgttcatgtcatgtctaaacatactgacttttcaagtgaaatcataggatacaaggctattgatggtggtgtcactcgtgacatagcatctacagatacttgttttgctaacaaacatgctgattttgacacatggtttagccagcgtggtggtagttatactaatgacaaagcttgcccattgattgctgcagtcataacaagagaagtgggttttgtcgtgcctggtttgcctggcacgatattacgcacaactaatggtgactttttgcatttcttacctagagtttttagtgcagttggtaacatctgttacacaccatcaaaacttatagagtacactgactttgcaacatcagcttgtgttttggctgctgaatgtacaatttttaaagatgcttctggtaagccagtaccatattgttatgataccaatgtactagaaggttctgttgcttatgaaagtttacgccctgacacacgttatgtgctcatggatggctctattattcaatttcctaacacctaccttgaaggttctgttagagtggtaacaacttttgattctgagtactgtaggcacggcacttgtgaaagatcagaagctggtgtttgtgtatctactagtggtagatgggtacttaacaatgattattacagatctttaccaggagttttctgtggtgtagatgctgtaaatttacttactaatatgtttacaccactaattcaacctattggtgctttggacatatcagcatctatagtagctggtggtattgtagctatcgtagtaacatgccttgcctactattttatgaggtttagaagagcttttggtgaatacagtcatgtagttgcctttaatactttactattccttatgtcattcactgtactctgtttaacaccagtttactcattcttacctggtgtttattctgttatttacttgtacttgacattttatcttactaatgatgtttcttttttagcacatattcagtggatggttatgttcacacctttagtacctttctggataacaattgcttatatcatttgtatttccacaaagcatttctattggttctttagtaattacctaaagagacgtgtagtctttaatggtgtttcctttagtacttttgaagaagctgcgctgtgcacctttttgttaaataaagaaatgtatctaaagttgcgtagtgatgtgctattacctcttacgcaatataatagatacttagctctttataataagtacaagtattttagtggagcaatggatacaactagctacagagaagctgcttgttgtcatctcgcaaaggctctcaatgacttcagtaactcaggttctgatgttctttaccaaccaccacaaacctctatcacctcagctgttttgcagagtggttttagaaaaatggcattcccatctggtaaagttgagggttgtatggtacaagtaacttgtggtacaactacacttaacggtctttggcttgatgacgtagtttactgtccaagacatgtgatctgcacctctgaagacatgcttaaccctaattatgaagatttactcattcgtaagtctaatcataatttcttggtacaggctggtaatgttcaactcagggttattggacattctatgcaaaattgtgtacttaagcttaaggttgatacagccaatcctaagacacctaagtataagtttgttcgcattcaaccaggacagactttttcagtgttagcttgttacaatggttcaccatctggtgtttaccaatgtgctatgaggcccaatttcactattaagggttcattccttaatggttcatgtggtagtgttggttttaacatagattatgactgtgtctctttttgttacatgcaccatatggaattaccaactggagttcatgctggcacagacttagaaggtaacttttatggaccttttgttgacaggcaaacagcacaagcagctggtacggacacaactattacagttaatgttttagcttggttgtacgctgctgttataaatggagacaggtggtttctcaatcgatttaccacaactcttaatgactttaaccttgtggctatgaagtacaattatgaacctctaacacaagaccatgttgacatactaggacctctttctgctcaaactggaattgccgttttagatatgtgtgcttcattaaaagaattactgcaaaatggtatgaatggacgtaccatattgggtagtgctttattagaagatgaatttacaccttttgatgttgttagacaatgctcaggtgttactttccaaagtgcagtgaaaagaacaatcaagggtacacaccactggttgttactcacaattttgacttcacttttagttttagtccagagtactcaatggtctttgttcttttttttgtatgaaaatgcctttttaccttttgctatgggtattattgctatgtctgcttttgcaatgatgtttgtcaaacataagcatgcatttctctgtttgtttttgttaccttctcttgccactgtagcttattttaatatggtctatatgcctgctagttgggtgatgcgtattatgacatggttggatatggttgatactagtttgtctggttttaagctaaaagactgtgttatgtatgcatcagctgtagtgttactaatccttatgacagcaagaactgtgtatgatgatggtgctaggagagtgtggacacttatgaatgtcttgacactcgtttataaagtttattatggtaatgctttagatcaagccatttccatgtgggctcttataatctctgttacttctaactactcaggtgtagttacaactgtcatgtttttggccagaggtattgtttttatgtgtgttgagtattgccctattttcttcataactggtaatacacttcagtgtataatgctagtttattgtttcttaggctatttttgtacttgttactttggcctcttttgtttactcaaccgctactttagactgactcttggtgtttatgattacttagtttctacacaggagtttagatatatgaattcacagggactactcccacccaagaatagcatagatgccttcaaactcaacattaaattgttgggtgttggtggcaaaccttgtatcaaagtagccactgtacagtctaaaatgtcagatgtaaagtgcacatcagtagtcttactctcagttttgcaacaactcagagtagaatcatcatctaaattgtgggctcaatgtgtccagttacacaatgacattctcttagctaaagatactactgaagcctttgaaaaaatggtttcactactttctgttttgctttccatgcagggtgctgtagacataaacaagctttgtgaagaaatgctggacaacagggcaaccttacaagctatagcctcagagtttagttcccttccatcatatgcagcttttgctactgctcaagaagcttatgagcaggctgttgctaatggtgattctgaagttgttcttaaaaagttgaagaagtctttgaatgtggctaaatctgaatttgaccgtgatgcagccatgcaacgtaagttggaaaagatggctgatcaagctatgacccaaatgtataaacaggctagatctgaggacaagagggcaaaagttactagtgctatgcagacaatgcttttcactatgcttagaaagttggataatgatgcactcaacaacattatcaacaatgcaagagatggttgtgttcccttgaacataatacctcttacaacagcagccaaactaatggttgtcataccagactataacacatataaaaatacgtgtgatggtacaacatttacttatgcatcagcattgtgggaaatccaacaggttgtagatgcagatagtaaaattgttcaacttagtgaaattagtatggacaattcacctaatttagcatggcctcttattgtaacagctttaagggccaattctgctgtcaaattacagaataatgagcttagtcctgttgcactacgacagatgtcttgtgctgccggtactacacaaactgcttgcactgatgacaatgcgttagcttactacaacacaacaaagggaggtaggtttgtacttgcactgttatccgatttacaggatttgaaatgggctagattccctaagagtgatggaactggtactatctatacagaactggaaccaccttgtaggtttgttacagacacacctaaaggtcctaaagtgaagtatttatactttattaaaggattaaacaacctaaatagaggtatggtacttggtagtttagctgccacagtacgtctacaagctggtaatgcaacagaagtgcctgccaattcaactgtattatctttctgtgcttttgctgtagatgctgctaaagcttacaaagattatctagctagtgggggacaaccaatcactaattgtgttaagatgttgtgtacacacactggtactggtcaggcaataacagttacaccggaagccaatatggatcaagaatcctttggtggtgcatcgtgttgtctgtactgccgttgccacatagatcatccaaatcctaaaggattttgtgacttaaaaggtaagtatgtacaaatacctacaacttgtgctaatgaccctgtgggttttacacttaaaaacacagtctgtaccgtctgcggtatgtggaaaggttatggctgtagttgtgatcaactccgcgaacccatgcttcagtcagctgatgcacaatcgtttttaaacgggtttgcggtgtaagtgcagcccgtcttacaccgtgcggcacaggcactagtactgatgtcgtatacagggcttttgacatctacaatgataaagtagctggttttgctaaattcctaaaaactaattgttgtcgcttccaagaaaaggacgaagatgacaatttaattgattcttactttgtagttaagagacacactttctctaactaccaacatgaagaaacaatttataatttacttaaggattgtccagctgttgctaaacatgacttctttaagtttagaatagacggtgacatggtaccacatatatcacgtcaacgtcttactaaatacacaatggcagacctcgtctatgctttaaggcattttgatgaaggtaattgtgacacattaaaagaaatacttgtcacatacaattgttgtgatgatgattatttcaataaaaaggactggtatgattttgtagaaaacccagatatattacgcgtatacgccaacttaggtgaacgtgtacgccaagctttgttaaaaacagtacaattctgtgatgccatgcgaaatgctggtattgttggtgtactgacattagataatcaagatctcaatggtaactggtatgatttcggtgatttcatacaaaccacgccaggtagtggagttcctgttgtagattcttattattcattgttaatgcctatattaaccttgaccagggctttaactgcagagtcacatgttgacactgacttaacaaagccttacattaagtgggatttgttaaaatatgacttcacggaagagaggttaaaactctttgaccgttattttaaatattgggatcagacataccacccaaattgtgttaactgtttggatgacagatgcattctgcattgtgcaaactttaatgttttattctctacagtgttcccacctacaagttttggaccactagtgagaaaaatatttgttgatggtgttccatttgtagtttcaactggataccacttcagagagctaggtgttgtacataatcaggatgtaaacttacatagctctagacttagttttaaggaattacttgtgtatgctgctgaccctgctatgcacgctgcttctggtaatctattactagataaacgcactacgtgcttttcagtagctgcacttactaacaatgttgcttttcaaactgtcaaacccggtaattttaacaaagacttctatgactttgctgtgtctaagggtttctttaaggaaggaagttctgttgaattaaaacacttcttctttgctcaggatggtaatgctgctatcagcgattatgactactatcgttataatctaccaacaatgtgtgatatcagacaactactatttgtagttgaagttgttgataagtactttgattgttacgatggtggctgtattaatgctaaccaagtcatcgtcaacaacctagacaaatcagctggttttccatttaataaatggggtaaggctagactttattatgattcaatgagttatgaggatcaagatgcacttttcgcatatacaaaacgtaatgtcatccctactataactcaaatgaatcttaagtatgccattagtgcaaagaatagagctcgcaccgtagctggtgtctctatctgtagtactatgaccaatagacagtttcatcaaaaattattgaaatcaatagccgccactagaggagctactgtagtaattggaacaagcaaattctatggtggttggcacaacatgttaaaaactgtttatagtgatgtagaaaaccctcaccttatgggttgggattatcctaaatgtgatagagccatgcctaacatgcttagaattatggcctcacttgttcttgctcgcaaacatacaacgtgttgtagcttgtcacaccgtttctatagattagctaatgagtgtgctcaagtattgagtgaaatggtcatgtgtggcggttcactatatgttaaaccaggtggaacctcatcaggagatgccacaactgcttatgctaatagtgtttttaacatttgtcaagctgtcacggccaatgttaatgcacttttatctactgatggtaacaaaattgccgataagtatgtccgcaatttacaacacagactttatgagtgtctctatagaaatagagatgttgacacagactttgtgaatgagttttacgcatatttgcgtaaacatttctcaatgatgatactctctgacgatgctgttgtgtgtttcaatagcacttatgcatctcaaggtctagtggctagcataaagaactttaagtcagttctttattatcaaaacaatgtttttatgtctgaagcaaaatgttggactgagactgaccttactaaaggacctcatgaattttgctctcaacatacaatgctagttaaacagggtgatgattatgtgtaccttccttacccagatccatcaagaatcctaggggccggctgttttgtagatgatatcgtaaaaacagatggtacacttatgattgaacggttcgtgtctttagctatagatgcttacccacttactaaacatcctaatcaggagtatgctgatgtctttcatttgtacttacaatacataagaaagctacatgatgagttaacaggacacatgttagacatgtattctgttatgcttactaatgataacacttcaaggtattgggaacctgagttttatgaggctatgtacacaccgcatacagtcttacaggctgttggggcttgtgttctttgcaattcacagacttcattaagatgtggtgcttgcatacgtagaccattcttatgttgtaaatgctgttacgaccatgtcatatcaacatcacataaattagtcttgtctgttaatccgtatgtttgcaatgctccaggttgtgatgtcacagatgtgactcaactttacttaggaggtatgagctattattgtaaatcacataaaccacccattagttttccattgtgtgctaatggacaagtttttggtttatataaaaatacatgtgttggtagcgataatgttactgactttaatgcaattgcaacatgtgactggacaaatgctggtgattacattttagctaacacctgtactgaaagactcaagctttttgcagcagaaacgctcaaagctactgaggagacatttaaactgtcttatggtattgctactgtacgtgaagtgctgtctgacagagaattacatctttcatgggaagttggtaaacctagaccaccacttaaccgaaattatgtctttactggttatcgtgtaactaaaaacagtaaagtacaaataggagagtacacctttgaaaaaggtgactatggtgatgctgttgtttaccgaggtacaacaacttacaaattaaatgttggtgattattttgtgctgacatcacatacagtaatgccattaagtgcacctacactagtgccacaagagcactatgttagaattactggcttatacccaacactcaatatctcagatgagttttctagcaatgttgcaaattatcaaaaggttggtatgcaaaagtattctacactccagggaccacctggtactggtaagagtcattttgctattggcctagctctctactacccttctgctcgcatagtgtatacagcttgctctcatgccgctgttgatgcactatgtgagaaggcattaaaatatttgcctatagataaatgtagtagaattatacctgcacgtgctcgtgtagagtgttttgataaattcaaagtgaattcaacattagaacagtatgtcttttgtactgtaaatgcattgcctgagacgacagcagatatagttgtctttgatgaaatttcaatggccacaaattatgatttgagtgttgtcaatgccagattacgtgctaagcactatgtgtacattggcgaccctgctcaattacctgcaccacgcacattgctaactaagggcacactagaaccagaatatttcaattcagtgtgtagacttatgaaaactataggtccagacatgttcctcggaacttgtcggcgttgtcctgctgaaattgttgacactgtgagtgctttggtttatgataataagcttaaagcacataaagacaaatcagctcaatgctttaaaatgttttataagggtgttatcacgcatgatgtttcatctgcaattaacaggccacaaataggcgtggtaagagaattccttacacgtaaccctgcttggagaaaagctgtctttatttcaccttataattcacagaatgctgtagcctcaaagattttgggactaccaactcaaactgttgattcatcacagggctcagaatatgactatgtcatattcactcaaaccactgaaacagctcactcttgtaatgtaaacagatttaatgttgctattaccagagcaaaagtaggcatactttgcataatgtctgatagagacctttatgacaagttgcaatttacaagtcttgaaattccacgtaggaatgtggcaactttacaagctgaaaatgtaacaggactctttaaagattgtagtaaggtaatcactgggttacatcctacacaggcacctacacacctcagtgttgacactaaattcaaaactgaaggtttatgtgttgacatacctggcatacctaaggacatgacctatagaagactcatctctatgatgggttttaaaatgaattatcaagttaatggttaccctaacatgtttatcacccgcgaagaagctataagacatgtacgtgcatggattggcttcgatgtcgaggggtgtcatgctactagagaagctgttggtaccaatttacctttacagctaggtttttctacaggtgttaacctagttgctgtacctacaggttatgttgatacacctaataatacagatttttccagagttagtgctaaaccaccgcctggagatcaatttaaacacctcataccacttatgtacaaaggacttctttggaatgtagtgcgtataaagattgtacaaatgttaagtgacacacttaaaaatctctctgacagagtcgtatttgtcttatgggcacatggctttgagttgacatctatgaagtattttgtgaaaataggacctgagcgcacctgttgtctatgtgatagacgtgccacatgcttttccactgcttcagacacttatgcctgttggcatcattctattggatttgattacgtctataatccgtttatgattgatgttcaacaatggggttttacaggtaacctacaaagcaaccatgatctgtattgtcaagtccatggtaatgcacatgtagctagttgtgatgcaatcatgactaggtgtctagctgtccacgagtgctttgttaagcgtgttgactggactattgaatatcctataattggtgatgaactgaagattaatgcggcttgtagaaaggttcaacacatggttgttaaagctgcattattagcagacaaattcccagttcttcacgacattggtaaccctaaagctattaagtgtgtacctcaagctgatgtagaatggaagttctatgatgcacagccttgtagtgacaaagcttataaaatagaagaattattctattcttatgccacacattctgacaaattcacagatggtgtatgcctattttggaattgcaatgtcgatagatatcctgctaattccattgtttgtagatttgacactagagtgctatctaaccttaacttgcctggttgtgatggtggcagtttgtatgtaaataaacatgcattccacacaccagcttttgataaaagtgcttttgttaatttaaaacaattaccatttttctattactctgacagtccatgtgagtctcatggaaaacaagtagtgtcagatatagattatgtaccactaaagtctgctacgtgtataacacgttgcaatttaggtggtgctgtctgtagacatcatgctaatgagtacagattgtatctcgatgcttataacatgatgatctcagctggctttagcttgtgggtttacaaacaatttgatacttataacctctggaacacttttacaagacttcagagtttagaaaatgtggcttttaatgttgtaaataagggacactttgatggacaacagggtgaagtaccagtttctatcattaataacactgtttacacaaaagttgatggtgttgatgtagaattgtttgaaaataaaacaacattacctgttaatgtagcatttgagctttgggctaagcgcaacattaaaccagtaccagaggtgaaaatactcaataatttgggtgtggacattgctgctaatactgtgatctgggactacaaaagagatgctccagcacatatatctactattggtgtttgttctatgactgacatagccaagaaaccaactgaaacgatttgtgcaccactcactgtcttttttgatggtagagttgatggtcaagtagacttatttagaaatgcccgtaatggtgttcttattacagaaggtagtgttaaaggtttacaaccatctgtaggtcccaaacaagctagtcttaatggagtcacattaattggagaagccgtaaaaacacagttcaattattataagaaagttgatggtgttgtccaacaattacctgaaacttactttactcagagtagaaatttacaagaatttaaacccaggagtcaaatggaaattgatttcttagaattagctatggatgaattcattgaacggtataaattagaaggctatgccttcgaacatatcgtttatggagattttagtcatagtcagttaggtggtttacatctactgattggactagctaaacgttttaaggaatcaccttttgaattagaagattttattcctatggacagtacagttaaaaactatttcataacagatgcgcaaacaggttcatctaagtgtgtgtgttctgttattgatttattacttgatgattttgttgaaataataaaatcccaagatttatctgtagtttctaaggttgtcaaagtgactattgactatacagaaatttcatttatgctttggtgtaaagatggccatgtagaaacattttacccaaaattacaatctagtcaagcgtggcaaccgggtgttgctatgcctaatctttacaaaatgcaaagaatgctattagaaaagtgtgaccttcaaaattatggtgatagtgcaacattacctaaaggcataatgatgaatgtcgcaaaatatactcaactgtgtcaatatttaaacacattaacattagctgtaccctataatatgagagttatacattttggtgctggttctgataaaggagttgcaccaggtacagctgttttaagacagtggttgcctacgggtacgctgcttgtcgattcagatcttaatgactttgtctctgatgcagattcaactttgattggtgattgtgcaactgtacatacagctaataaatgggatctcattattagtgatatgtacgaccctaagactaaaaatgttacaaaagaaaatgactctaaagagggttttttcacttacatttgtgggtttatacaacaaaagctagctcttggaggttccgtggctataaagataacagaacattcttggaatgctgatctttataagctcatgggacacttcgcatggtggacagcctttgttactaatgtgaatgcgtcatcatctgaagcatttttaattggatgtaattatcttggcaaaccacgcgaacaaatagatggttatgtcatgcatgcaaattacatattttggaggaatacaaatccaattcagttgtcttcctattctttatttgacatgagtaaatttccccttaaattaaggggtactgctgttatgtctttaaaagaaggtcaaatcaatgatatgattttatctcttcttagtaaaggtagacttataattagagaaaacaacagagttgttatttctagtgatgttcttgttaacaactaaacgaacaatgtttgtttttcttgttttattgccactagtctctagtcagtgtgttaatcttacaaccagaactcaattaccccctgcatacactaattctttcacacgtggtgtttattaccctgacaaagttttcagatcctcagttttacattcaactcaggacttgttcttacctttcttttccaatgttacttggttccatgctatacatgtctctgggaccaatggtactaagaggtttgataaccctgtcctaccatttaatgatggtgtttattttgcttccactgagaagtctaacataataagaggctggatttttggtactactttagattcgaagacccagtccctacttattgttaataacgctactaatgttgttattaaagtctgtgaatttcaattttgtaatgatccatttttgggtgtttattaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcaggatgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtgcaggtatatgcgctagttatcagactcagactaattctcctcggcgggcacgtagtgtagctagtcaatccatcattgcctacactatgtcacttggtgcagaaaattcagttgcttactctaataactctattgccatacccacaaattttactattagtgttaccacagaaattctaccagtgtctatgaccaagacatcagtagattgtacaatgtacatttgtggtgattcaactgaatgcagcaatcttttgttgcaatatggcagtttttgtacacaattaaaccgtgctttaactggaatagctgttgaacaagacaaaaacacccaagaagtttttgcacaagtcaaacaaatttacaaaacaccaccaattaaagattttggtggttttaatttttcacaaatattaccagatccatcaaaaccaagcaagaggtcatttattgaagatctacttttcaacaaagtgacacttgcagatgctggcttcatcaaacaatatggtgattgccttggtgatattgctgctagagacctcatttgtgcacaaaagtttaacggccttactgttttgccacctttgctcacagatgaaatgattgctcaatacacttctgcactgttagcgggtacaatcacttctggttggacctttggtgcaggtgctgcattacaaataccatttgctatgcaaatggcttataggtttaatggtattggagttacacagaatgttctctatgagaaccaaaaattgattgccaaccaatttaatagtgctattggcaaaattcaagactcactttcttccacagcaagtgcacttggaaaacttcaagatgtggtcaaccaaaatgcacaagctttaaacacgcttgttaaacaacttagctccaattttggtgcaatttcaagtgttttaaatgatatcctttcacgtcttgacaaagttgaggctgaagtgcaaattgataggttgatcacaggcagacttcaaagtttgcagacatatgtgactcaacaattaattagagctgcagaaatcagagcttctgctaatcttgctgctactaaaatgtcagagtgtgtacttggacaatcaaaaagagttgatttttgtggaaagggctatcatcttatgtccttccctcagtcagcacctcatggtgtagtcttcttgcatgtgacttatgtccctgcacaagaaaagaacttcacaactgctcctgccatttgtcatgatggaaaagcacactttcctcgtgaaggtgtctttgtttcaaatggcacacactggtttgtaacacaaaggaatttttatgaaccacaaatcattactacagacaacacatttgtgtctggtaactgtgatgttgtaataggaattgtcaacaacacagtttatgatcctttgcaacctgaattagactcattcaaggaggagttagataaatattttaagaatcatacatcaccagatgttgatttaggtgacatctctggcattaatgcttcagttgtaaacattcaaaaagaaattgaccgcctcaatgaggttgccaagaatttaaatgaatctctcatcgatctccaagaacttggaaagtatgagcagtatataaaatggccatggtacatttggctaggttttatagctggcttgattgccatagtaatggtgacaattatgctttgctgtatgaccagttgctgtagttgtctcaagggctgttgttcttgtggatcctgctgcaaatttgatgaagacgactctgagccagtgctcaaaggagtcaaattacattacacataaacgaacttatggatttgtttatgagaatcttcacaattggaactgtaactttgaagcaaggtgaaatcaaggatgctactccttcagattttgttcgcgctactgcaacgataccgatacaagcctcactccctttcggatggcttattgttggcgttgcacttcttgctgtttttcagagcgcttccaaaatcataaccctcaaaaagagatggcaactagcactctccaagggtgttcactttgtttgcaacttgctgttgttgtttgtaacagtttactcacaccttttgctcgttgctgctggccttgaagccccttttctctatctttatgctttagtctacttcttgcagagtataaactttgtaagaataataatgaggctttggctttgctggaaatgccgttccaaaaacccattactttatgatgccaactattttctttgctggcatactaattgttacgactattgtataccttacaatagtgtaacttcttcaattgtcattacttcaggtgatggcacaacaagtcctatttctgaacatgactaccagattggtggttatactgaaaaatgggaatctggagtaaaagactgtgttgtattacacagttacttcacttcagactattaccagctgtactcaactcaattgagtacagacactggtgttgaacatgttaccttcttcatctacaataaaattgttgatgagcctgaagaacatgtccaaattcacacaatcgacggttcatccggagttgttaatccagtaatggaaccaatttatgatgaaccgacgacgactactagcgtgcctttgtaagcacaagctgatgagtacgaacttatgtactcattcgtttcggaagagacaggtacgttaatagttaatagcgtacttctttttcttgctttcgtggtattcttgctagttacactagccatccttactgcgcttcgattgtgtgcgtactgctgcaatattgttaacgtgagtcttgtaaaaccttctttttacgtttactctcgtgttaaaaatctgaattcttctagagttcctgatcttctggtctaaacgaactaaatattatattagtttttctgtttggaactttaattttagccatggcagattccaacggtactattaccgttgaagagcttaaaaagctccttgaacaatggaacctagtaataggtttcctattccttacatggatttgtcttctacaatttgcctatgccaacaggaataggtttttgtatataattaagttaattttcctctggctgttatggccagtaactttagcttgttttgtgcttgctgctgtttacagaataaattggatcaccggtggaattgctatcgcaatggcttgtcttgtaggcttgatgtggctcagctacttcattgcttctttcagactgtttgcgcgtacgcgttccatgtggtcattcaatccagaaactaacattcttctcaacgtgccactccatggcactattctgaccagaccgcttctagaaagtgaactcgtaatcggagctgtgatccttcgtggacatcttcgtattgctggacaccatctaggacgctgtgacatcaaggacctgcctaaagaaatcactgttgctacatcacgaacgctttcttattacaaattgggagcttcgcagcgtgtagcaggtgactcaggttttgctgcatacagtcgctacaggattggcaactataaattaaacacagaccattccagtagcagtgacaatattgctttgcttgtacagtaagtgacaacagatgtttcatctcgttgactttcaggttactatagcagagatattactaattattatgaggacttttaaagtttccatttggaatcttgattacatcataaacctcataattaaaaatttatctaagtcactaactgagaataaatattctcaattagatgaagagcaaccaatggagattgattaaacgaacatgaaaattattcttttcttggcactgataacactcgctacttgtgagctttatcactaccaagagtgtgttagaggtacaacagtacttttaaaagaaccttgctcttctggaacatacgagggcaattcaccatttcatcctctagctgataacaaatttgcactgacttgctttagcactcaatttgcttttgcttgtcctgacggcgtaaaacacgtctatcagttacgtgccagatcagtttcacctaaactgttcatcagacaagaggaagttcaagaactttactctccaatttttcttattgttgcggcaatagtgtttataacactttgcttcacactcaaaagaaagacagaatgattgaactttcattaattgacttctatttgtgctttttagcctttctgctattccttgttttaattatgcttattatcttttggttctcacttgaactgcaagatcataatgaaacttgtcacgcctaaacgaacatgaaatttcttgttttcttaggaatcatcacaactgtagctgcatttcaccaagaatgtagtttacagtcatgtactcaacatcaaccatatgtagttgatgacccgtgtcctattcacttctattctaaatggtatattagagtaggagctagaaaatcagcacctttaattgaattgtgcgtggatgaggctggttctaaatcacccattcagtacatcgatatcggtaattatacagtttcctgtttaccttttacaattaattgccaggaacctaaattgggtagtcttgtagtgcgttgttcgttctatgaagactttttagagtatcatgacgttcgtgttgttttagatttcatctaaacgaacaaactaaaatgtctgataatggaccccaaaatcagcgaaatgcaccccgcattacgtttggtggaccctcagattcaactggcagtaaccagaatggagaacgcagtggggcgcgatcaaaacaacgtcggccccaaggtttacccaataatactgcgtcttggttcaccgctctcactcaacatggcaaggaagaccttaaattccctcgaggacaaggcgttccaattaacaccaatagcagtccagatgaccaaattggctactaccgaagagctaccagacgaattcgtggtggtgacggtaaaatgaaagatctcagtccaagatggtatttctactacctaggaactgggccagaagctggacttccctatggtgctaacaaagacggcatcatatgggttgcaactgagggagccttgaatacaccaaaagatcacattggcacccgcaatcctgctaacaatgctgcaatcgtgctacaacttcctcaaggaacaacattgccaaaaggcttctacgcagaagggagcagaggcggcagtcaagcctcttctcgttcctcatcacgtagtcgcaacagttcaagaaattcaactccaggcagcagtaggggaacttctcctgctagaatggctggcaatggcggtgatgctgctcttgctttgctgctgcttgacagattgaaccagcttgagagcaaaatgtctggtaaaggccaacaacaacaaggccaaactgtcactaagaaatctgctgctgaggcttctaagaagcctcggcaaaaacgtactgccactaaagcatacaatgtaacacaagctttcggcagacgtggtccagaacaaacccaaggaaattttggggaccaggaactaatcagacaaggaactgattacaaacattggccgcaaattgcacaatttgcccccagcgcttcagcgttcttcggaatgtcgcgcattggcatggaagtcacaccttcgggaacgtggttgacctacacaggtgccatcaaattggatgacaaagatccaaatttcaaagatcaagtcattttgctgaataagcatattgacgcatacaaaacattcccaccaacagagcctaaaaaggacaaaaagaagaaggctgatgaaactcaagccttaccgcagagacagaagaaacagcaaactgtgactcttcttcctgctgcagatttggatgatttctccaaacaattgcaacaatccatgagcagtgctgactcaactcaggcctaaactcatgcagaccacacaaggcagatgggctatataaacgttttcgcttttccgtttacgatatatagtctactcttgtgcagaatgaattctcgtaactacatagcacaagtagatgtagttaactttaatctcacatagcaatctttaatcagtgtgtaacattagggaggacttgaaagagccaccacattttcaccgaggccacgcggagtacgatcgagtgtacagtgaacaatgctagggagagctgcctatatggaagagccctaatgtgtaaaattaattttagtagtgctatccccatgtgattttaatagcttcttaggagaatgacaaaaa

SEQ ID NO: 2 - protein, 1273 aa, unidentified
  REGION 1..12: note = MISC_FEATURE - Signal peptide
  REGION 13..685: note = MISC_FEATURE - S1 region
  REGION 13..1213: note = MISC_FEATURE - Extracellular domain
  REGION 686..1273: note = MISC_FEATURE - S2 region
  REGION 1214..1234: note = MISC_FEATURE - Cytoplasmic domain
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 3 - protein, 75 aa, unidentified
MYSFVSEETGTLIVNSVLLFLAFVVFLLVTLAILTALRLCAYCCNIVNVSLVKPSFYVYSRVKNLNSSRVPDLLV

SEQ ID NO: 4 - protein, 196 aa, unidentified
CPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKST

SEQ ID NO: 5 - protein, 202 aa, synthetic construct
  REGION 1..202: note = Truncated spike (S) protein w/ N-terminal C6
CCCCCCCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKST

SEQ ID NO: 6 - protein, 201 aa, synthetic construct
  REGION 1..201: note = Truncated spike (S) protein w/ C-terminal H6
PFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTHHHHHH

SEQ ID NO: 7 - protein, 9 aa, unidentified
LLFNKVTLA

SEQ ID NO: 8 - protein, 13 aa, unidentified
KMSECVLGQSKRV

SEQ ID NO: 9 - protein, 9 aa, unidentified
YLQPRTFLL

SEQ ID NO: 10 - protein, 9 aa, synthetic construct
  REGION 1..9: note = T cell epitope
KLWAQCVQL

SEQ ID NO: 11 - protein, 9 aa, synthetic construct
  REGION 1..9: note = T cell epitope
LLYDANYFL

SEQ ID NO: 12 - protein, 12 aa, synthetic construct
  REGION 1..12: note = T cell epitope
PRWYFYYLGTGP

SEQ ID NO: 13 - protein, 9 aa, synthetic construct
  REGION 1..9: note = T cell epitope
SPRWYFYYL

SEQ ID NO: 14 - protein, 8 aa, synthetic construct
  REGION 1..8: note = T cell epitope
WSFNPETN

SEQ ID NO: 15 - protein, 9 aa, synthetic construct
  REGION 1..9: note = T cell epitope
QPPGTGKSH

SEQ ID NO: 16 - protein, 17 aa, synthetic construct
  REGION 1..17: note = T cell epitope
VYTACSHAAVDALCEKA

SEQ ID NO: 17 - protein, 9 aa, synthetic construct
  REGION 1..9: note = T cell epitope
KTFPPTEPK

SEQ ID NO: 18 - protein, 10 aa, synthetic construct
  REGION 1..10: note = T cell epitope
CTDDNALAYY

SEQ ID NO: 19 - protein, 10 aa, synthetic construct
  REGION 1..10: note = T cell epitope
TTDPSFLGRY

SEQ ID NO: 20 - protein, 9 aa, synthetic construct
  REGION 1..9: note = T cell epitope
FTSDYYQLY

SEQ ID NO: 21 - protein, 9 aa, unidentified
FVFLVLLPL

SEQ ID NO: 22 - protein, 9 aa, unidentified
FIAGLIAIV

SEQ ID NO: 23 - protein, 18 aa, unidentified
PINLVRDLPQGFSALEPL

SEQ ID NO: 24 - protein, 18 aa, unidentified
PATVCGPKKSTNLVKNKC

SEQ ID NO: 25 - protein, 18 aa, unidentified
TESNKKFLPFQQFGRDIA

SEQ ID NO: 26 - protein, 18 aa, unidentified
GIAVEQDKNTQEVFAQVK

SEQ ID NO: 27 - protein, 18 aa, unidentified
NTQEVFAQVKQIYKTPPI

SEQ ID NO: 28 - protein, 18 aa, unidentified
VKQIYKTPPIKDFGGFNF

SEQ ID NO: 29 - protein, 18 aa, unidentified
PIKDFGGFNFSQILPDPS

SEQ ID NO: 30 - protein, 18 aa, unidentified
PSKPSKRSFIEDLLFNKV

SEQ ID NO: 31 - protein, 14 aa, unidentified
LTTRTQLPPAYTNS

SEQ ID NO: 32 - protein, 8 aa, unidentified
TNGTKRFD

SEQ ID NO: 33 - protein, 5 aa, unidentified
ASTEK

SEQ ID NO: 34 - protein, 6 aa, unidentified
LDSKTQ

SEQ ID NO: 35 - protein, 10 aa, unidentified
YYHKNNKSWM

SEQ ID NO: 36 - protein, 4 aa, unidentified
SEFR

SEQ ID NO: 37 - protein, 6 aa, unidentified
IYSKHT

SEQ ID NO: 38 - protein, 6 aa, unidentified
TPGDSS

SEQ ID NO: 39 - protein, 7 aa, unidentified
KYNENGT

SEQ ID NO: 40 - protein, 10 aa, unidentified
QTSNFRVQPT

SEQ ID NO: 41 - protein, 6 aa, unidentified
AWNRKR

SEQ ID NO: 42 - protein, 6 aa, unidentified
NSNNLD

SEQ ID NO: 43 - protein, 8 aa, unidentified
LKPFERDI

SEQ ID NO: 44 - protein, 5 aa, unidentified
GFQPT

SEQ ID NO: 45 - protein, 4 aa, unidentified
YQPY

SEQ ID NO: 46 - protein, 4 aa, unidentified
PKKS

SEQ ID NO: 47 - protein, 5 aa, unidentified
ESNKK

SEQ ID NO: 48 - protein, 13 aa, unidentified
IADTTDAVRDPQT

SEQ ID NO: 49 - protein, 6 aa, unidentified
GTNTSN

SEQ ID NO: 50 - protein, 10 aa, unidentified
DQLTPTWRVY

SEQ ID NO: 51 - protein, 6 aa, unidentified
HVNNSY

SEQ ID NO: 52 - protein, 12 aa, unidentified
YQTQTNSPRRAR

SEQ ID NO: 53 - protein, 5 aa, unidentified
SMTKT

SEQ ID NO: 54 - protein, 7 aa, unidentified
EQDKNTQ

SEQ ID NO: 55 - protein, 10 aa, unidentified
DPSKPSKRSF

SEQ ID NO: 56 - protein, 4 aa, unidentified
MAYR

SEQ ID NO: 57 - protein, 7 aa, unidentified
NVLYENQ

SEQ ID NO: 58 - protein, 4 aa, unidentified
QSKR

SEQ ID NO: 59 - protein, 5 aa, unidentified
FPQSA

SEQ ID NO: 60 - protein, 9 aa, unidentified
VPAQEKNFT

SEQ ID NO: 61 - protein, 9 aa, unidentified
KYFKNHTSP

SEQ ID NO: 62 - protein, 8 aa, unidentified
IQKEIDRL

SEQ ID NO: 63 - protein, 6 aa, unidentified
FDEDDS

SEQ ID NO: 64 - protein, 4 aa, synthetic construct
  REGION 1..4: note = B cell epitope
TTKR

SEQ ID NO: 65 - protein, 19 aa, unidentified
NSNNLDSKVGGNYNYLYRL

SEQ ID NO: 66 - protein, 35 aa, unidentified
YQAGSTPCNGVEGFNCYFPLQSYGFQPTNGVGYQP

SEQ ID NO: 67 - protein, 32 aa, unidentified
QILPDPSKPSKRSFIEDLLFNKVTLADAGFIK

SEQ ID NO: 68 - protein, 11 aa, unidentified
SFIEDLLFNKV

SEQ ID NO: 69 - protein, 28 aa, unidentified
ASANLAATKMSECVLGQSKRVDFCGKGY

SEQ ID NO: 70 - protein, 16 aa, unidentified
KMSECVLGQSKRVDFC

SEQ ID NO: 71 - protein, 13 aa, unidentified
KMSECVLGQSKRV

SEQ ID NO: 72 - protein, 79 aa, synthetic construct
  REGION 1..79: note = S protein scaffold sequence
VIAWNSNNLDSKVGGNYNYLYRLFRKSNLKPFERDISTEIYQAGSTPCNGVEGFNCYFPLQSYGFQPTNGVGYQPYRVV

SEQ ID NO: 73 - protein, 74 aa, synthetic construct
  REGION 1..74: note = S protein scaffold sequence
VIAWNSNNLDSKVGGNYNYLYRLKMSECVLGQSKRVQALLFNKVTLAGFNCYFPLQSYGFQPTNGVGYQPYRVV

SEQ ID NO: 74 - protein, 66 aa, synthetic construct
  REGION 1..66: note = S protein scaffold sequence
VIAWNSNNLDSKVGGNYNYLYRLGSGSGQAGSTPCNGVEGFNCYFPLQSYGFQPTNGVGYQPYRVV

SEQ ID NO: 75 - protein, 79 aa, synthetic construct
  REGION 1..79: note = S protein scaffold sequence
VIAWNSNNLDSKVGGNYNYLYRCFRKSNLKPFERDISTEIYQAGSTPGNGVEGFNGYFCLQSYGFQPTNGVGYQPYRVV

SEQ ID NO: 76 - protein, 74 aa, synthetic construct
  REGION 1..74: note = S protein scaffold sequence
VIAWNSNNLDSKVGGNYNYLYRCKMSECVLGQSKRVQALLFNKVTLAGFNGYFCLQSYGFQPTNGVGYQPYRVV

SEQ ID NO: 77 - protein, 66 aa, synthetic construct
  REGION 1..66: note = S protein scaffold sequence
VIAWNSNNLDSKVGGNYNYLYRCGSGSGQAGSTPGNGVEGFNGYFCLQSYGFQPTNGVGYQPYRVV

SEQ ID NO: 78 - protein, 84 aa, synthetic construct
  REGION 1..84: note = S protein scaffold sequence
EEVIAWNSNNLDSKVGGNYNYLYRCFRKSNLKPFERDISTEIYQAGSTPGNGVEGFNGYFCLQSYGFQPTNGVGYQPYRVVRRR

SEQ ID NO: 79 - protein, 79 aa, synthetic construct
  REGION 1..79: note = S protein scaffold sequence
EEVIAWNSNNLDSKVGGNYNYLYRCKMSECVLGQSKRVQALLFNKVTLAGFNGYFCLQSYGFQPTNGVGYQPYRVVRRR

SEQ ID NO: 80 - protein, 71 aa, synthetic construct
  REGION 1..71: note = S protein scaffold sequence
EEVIAWNSNNLDSKVGGNYNYLYRCGSGSGQAGSTPGNGVEGFNGYFCLQSYGFQPTNGVGYQPYRVVRRR

SEQ ID NO: 81 - protein, 72 aa, synthetic construct
  REGION 1..72: note = S protein scaffold sequence
NSNNLDSKVGGNYNYLYRLFRKSNLKPFERDISTEIYQAGSTPCNGVEGFNCYFPLQSYGFQPTNGVGYQPY

SEQ ID NO: 82 - protein, 60 aa, synthetic construct
  REGION 1..60: note = S protein scaffold sequence
NSNNLDSKVGGNYNYLYRLGSGSGSQAGSTPCNGVEGFNCYFPLQSYGFQPTNGVGYQPY

SEQ ID NO: 83 - protein, 59 aa, synthetic construct
  REGION 1..59: note = S protein scaffold sequence
NSNNLDSKVGGNYNYLYRLGSGSGQAGSTPCNGVEGFNCYFPLQSYGFQPTNGVGYQPY

SEQ ID NO: 84 - protein, 58 aa, synthetic construct
  REGION 1..58: note = S protein scaffold sequence
NSNNLDSKVGGNYNYLYRLGSGSQAGSTPCNGVEGFNCYFPLQSYGFQPTNGVGYQPY

SEQ ID NO: 85 - protein, 57 aa, synthetic construct
  REGION 1..57: note = S protein scaffold sequence
NSNNLDSKVGGNYNYLYRLGSGQAGSTPCNGVEGFNCYFPLQSYGFQPTNGVGYQPY

SEQ ID NO: 86 - protein, 67 aa, synthetic construct
  REGION 1..67: note = S protein scaffold sequence
NSNNLDSKVGGNYNYLYRLKMSECVLGQSKRVQALLFNKVTLAGFNCYFPLQSYGFQPTNGVGYQPY

SEQ ID NO: 87 - protein, 69 aa, synthetic construct
  REGION 1..69: note = S protein scaffold sequence
NSNNLDSKVGGNYNYLYRLFRKMSECVLGQSKRVQALLFNKVTLAGFNCYFPLQSYGFQPTNGVGYQPY

SEQ ID NO: 88 - protein, 71 aa, synthetic construct
  REGION 1..71: note = S protein scaffold sequence
NSNNLDSKVGGNYNYLYRLFRKSKMSECVLGQSKRVQALLFNKVTLAGFNCYFPLQSYGFQPTNGVGYQPY

SEQ ID NO: 89 - protein, 72 aa, synthetic construct
  REGION 1..72: note = S protein scaffold sequence
NSNNLDSKVGGNYNYLYRLFRKSNKMSECVLGQSKRVQALLFNKVTLAGFNCYFPLQSYGFQPTNGVGYQPY

SEQ ID NO: 90 - protein, 73 aa, synthetic construct
  REGION 1..73: note = S protein scaffold sequence
NSNNLDSKVGGNYNYLYRLFRKSNLKMSECVLGQSKRVQALLFNKVTLAGFNCYFPLQSYGFQPTNGVGYQPY

SEQ ID NO: 91 - protein, 74 aa, synthetic construct
  REGION 1..74: note = S protein scaffold sequence
NSNNLDSKVGGNYNYLYRLFRKSNLKKMSECVLGQSKRVQALLFNKVTLAGFNCYFPLQSYGFQPTNGVGYQPY

SEQ ID NO: 92 - protein, 79 aa, synthetic construct
  REGION 1..79: note = S protein scaffold sequence
VIAWNSRNLDSKVGGNYNYKYRLFRKSNLKPFERDISNEIYQAGSTPCNGVPGFNCYFPLQSYGFQPTTGVGYQPYRVV

SEQ ID NO: 93 - protein, 81 aa, synthetic construct
  REGION 1..81: note = S protein scaffold sequence
EEVIAWNSNNLDSKVGGNYNYLYRCKMSECVLGQSKRVQALLFNKVTLAQAGFNGYFCLQSYGFQPTNGVGYQPYRVVRRR

SEQ ID NO: 94 - protein, 84 aa, synthetic construct
  REGION 1..84: note = S protein scaffold sequence
EEVIAWNSNNLDSKVGGNYNYLYRCFRKSNLKPFERDISTEIYQAGSTPCNGVEGFNGYFCLQSYGFQPTNGVGYQPYRVVRRR

SEQ ID NO: 95 - protein, 85 aa, synthetic construct
  REGION 1..85: note = S protein scaffold sequence
EEVIAWNSNNLDSKVGGNYNYLYRCKMSECVLGQSKRVQALLFNKVTLAQAGFNGYFCLQSYGFQPTNGVGYQPYRVVRRREPEA

SEQ ID NO: 96 - protein, 85 aa, synthetic construct
  REGION 1..85: note = S protein scaffold sequence
EEVIAWNSNNLDSKVGGNYNYLYRCKMSESVLGQSKRVQALLFNKVTLAQAGFNGYFCLQSYGFQPTNGVGYQPYRVVRRREPEA

SEQ ID NO: 97 - protein, 85 aa, synthetic construct
  REGION 1..85: note = S protein scaffold sequence
EEVIAWNSNNLDSKVGGNYNYLYRLKMSECVLGQSKRVQALLFNKVTLAQAGFNGYFCLQSYGFQPTNGVGYQPYRVVRRREPEA

SEQ ID NO: 98 - protein, 101 aa, synthetic construct
  REGION 1..101: note = S protein scaffold sequence
EVEVEFEVEVIAWNSNNLDSKVGGNYNYLYRLFGSGSGSGSGSGSGSGSYQAGSTPCNGVEGFNSYFPLQSYGFQPTNGVGYQPYRVVRVRFRVRVREPEA

SEQ ID NO: 99 - protein, 101 aa, synthetic construct
  REGION 1..101: note = S protein scaffold sequence
EVEVEFEVEVIAWNSNNLDSKVGGNYNYLYRCFGSGSGSGSGSGSGSGSYQAGSTPCNGVEGFNSYFCLQSYGFQPTNGVGYQPYRVVRVRFRVRVREPEA

SEQ ID NO: 100 - protein, 112 aa, synthetic construct
  REGION 1..112: note = S protein scaffold sequence
EVEVEFEVEVIAWNSNNLDSKVGGNYNYLYRLFKLWAQCVQLYLQPRTFLLLLYDANYFLYQAGSTPCNGVEGFNSYFPLQSYGFQPTNGVGYQPYRVVRVRFRVRVREPEA

SEQ ID NO: 101 - protein, 112 aa, synthetic construct
  REGION 1..112: note = S protein scaffold sequence
EVEVEFEVEVIAWNSNNLDSKVGGNYNYLYRCFKLWAQCVQLYLQPRTFLLLLYDANYFLYQAGSTPCNGVEGFNSYFCLQSYGFQPTNGVGYQPYRVVRVRFRVRVREPEA

SEQ ID NO: 102 - protein, 104 aa, synthetic construct
  REGION 1..104: note = S protein scaffold sequence
CEVEVEFEVEVIAWNSRNLDSKVGGNYNYKYRLFKLWAQCVQLYLQPRTFLLLLYDANYFLYQAGSTPCNGVEGFNSYFPLQSYGFQPTTGVGYQPYRRREPEA

SEQ ID NO: 103 - protein, 93 aa, synthetic construct
  REGION 1..93: note = S protein scaffold sequence
CEVEVEFEVEVIAWNSRNLDSKVGGNYNYKYRLFGSGSGSGSGSGSGSGSYQAGSTPCNGVEGFNSYFPLQSYGFQPTTGVGYQPYRRREPEA

SEQ ID NO: 104 - protein, 107 aa, synthetic construct
  REGION 1..107: note = S protein scaffold sequence
CEVEVEFEVEVIAWNSRNLDSKVGGNYNYKYRLFKLWAQCVQLYLQPRTFLLLLYDANYFLNEIYQAGSTPCNGVEGFNSYFPLQSYGFQPTTGVGYQPYRRREPEA

SEQ ID NO: 105 - protein, 96 aa, synthetic construct
  REGION 1..96: note = S protein scaffold sequence
CEVEVEFEVEVIAWNSRNLDSKVGGNYNYKYRLFGSGSGSGSGSGSGSGSNEIYQAGSTPCNGVEGFNSYFPLQSYGFQPTTGVGYQPYRRREPEA

SEQ ID NO: 106 - protein, 110 aa, synthetic construct
  REGION 1..110: note = S protein scaffold sequence
EVEVEFEVEVIAWNSNNLDSKVGGNYNYLYRLFKMSESVLGQSKRVQALLFNKVTLAQYQAGSTPCNGVEGFNSYFPLQSYGFQPTNGVGYQPYRVVRVRFRVRVREPEA

SEQ ID NO: 107 - protein, 110 aa, synthetic construct
  REGION 1..110: note = S protein scaffold sequence
EVEVEFEVEVIAWNSNNLDSKVGGNYNYLYRCFKMSESVLGQSKRVQALLFNKVTLAQYQAGSTPCNGVEGFNSYFCLQSYGFQPTNGVGYQPYRVVRVRFRVRVREPEA

SEQ ID NO: 108 - protein, 102 aa, synthetic construct
  REGION 1..102: note = S protein scaffold sequence
CEVEVEFEVEVIAWNSRNLDSKVGGNYNYKYRLFKMSESVLGQSKRVQALLFNKVTLAQYQAGSTPCNGVEGFNSYFPLQSYGFQPTTGVGYQPYRRREPEA

SEQ ID NO: 109 - protein, 105 aa, synthetic construct
  REGION 1..105: note = S protein scaffold sequence
CEVEVEFEVEVIAWNSRNLDSKVGGNYNYKYRLFKMSESVLGQSKRVQALLFNKVTLAQNEIYQAGSTPCNGVEGFNSYFPLQSYGFQPTTGVGYQPYRRREPEA

SEQ ID NO: 110 - protein, 75 aa, synthetic construct
  REGION 1..75: note = S protein scaffold sequence
EEVIAWNSNNLDSKVGGNYNYLYRCGSGSGQAGSTPCNGVEGFNGYFCLQSYGFQPTNGVGYQPYRVVRRREPEA

SEQ ID NO: 111 - protein, 79 aa, synthetic construct
  REGION 1..79: note = S protein scaffold sequence
EEVIAWNSNNLDSKVGGNYNYLYRCKMSESVLGQSKRVQALLFNKVTLAGFNGYFCLQSYGFQPTNGVGYQPYRVVRRR

SEQ ID NO: 112 - protein, 66 aa, synthetic construct
  REGION 1..66: note = S protein scaffold sequence
VKAWNSNNLDSKVGGNYNYLYRLGSGSGQAGSTPCNGVEGFNCYFPLQSYGFQPTNGVGYQPYRVV

SEQ ID NO: 113 - protein, 66 aa, synthetic construct
  REGION 1..66: note = S protein scaffold sequence
VIKWNSNNLDSKVGGNYNYLYRLGSGSGQAGSTPCNGVEGFNCYFPLQSYGFQPTNGVGYQPYRVV

SEQ ID NO: 114 - protein, 57 aa, synthetic construct
  REGION 1..57: note = S protein scaffold sequence
SNNLDSKVGGNYNYLYRLGSGSGQAGSTPCNGVEGFNCYFPLQSYGFQPTNGVGYQP

SEQ ID NO: 115 - protein, 57 aa, synthetic construct
  REGION 1..57: note = S protein scaffold sequence
SNNLDSKVGGNYNYLYRCGSGSGQAGSTPGNGVEGFNGYFCLQSYGFQPTNGVGYQP

SEQ ID NO: 116 - protein, 59 aa, synthetic construct
  REGION 1..59: note = S protein scaffold sequence
SNNLDSKVGGNYNYLYRLFDGTEIYQAGSTPCNGVEGFNCYFPLQSYGFQPTNGVGYQP

SEQ ID NO: 117 - protein, 60 aa, synthetic construct
  REGION 1..60: note = S protein scaffold sequence
SNNLDSKVGGNYNYLYRLFNANDEIYQAGSTPCNGVEGFNCYFPLQSYGFQPTNGVGYQP

SEQ ID NO: 118 - protein, 60 aa, synthetic construct
  REGION 1..60: note = S protein scaffold sequence
SNNLDSKVGGNYNYLYRLFNAHDKIYQAGSTPCNGVEGFNCYFPLQSYGFQPTNGVGYQP

SEQ ID NO: 119 - protein, 60 aa, synthetic construct
  REGION 1..60: note = S protein scaffold sequence
SNNLDSKVGGNYNYLYRLFNANDKIYQAGSTPCNGVEGFNCYFPLQSYGFQPTNGVGYQP

SEQ ID NO: 120 - protein, 60 aa, synthetic construct
  REGION 1..60: note = S protein scaffold sequence
SNNLDSKVGGNYNYLYRLFDAHDKIYQAGSTPCNGVEGFNCYFPLQSYGFQPTNGVGYQP

SEQ ID NO: 121 - protein, 57 aa, synthetic construct
  REGION 1..57: note = S protein scaffold sequence
SNNLDSKVGGNYNYLYRLFPKPEQAGSTPCNGVEGFNCYFPLQSYGFQPTNGVGYQP

SEQ ID NO: 122 - protein, 59 aa, synthetic construct
  REGION 1..59: note = S protein scaffold sequence
SNNLDSKVGGNYNYLYRLFPGTEIYQAGSTPCNGVEGFNCYFPLQSYGFQPTNGVGYQP

SEQ ID NO: 123 - protein, 59 aa, synthetic construct
  REGION 1..59: note = S protein scaffold sequence
SNNLDSKVGGNYNYLYRLFPATEIYQAGSTPCNGVEGFNCYFPLQSYGFQPTNGVGYQP

SEQ ID NO: 124 - protein, 59 aa, synthetic construct
  REGION 1..59: note = S protein scaffold sequence
SNNLDSKVGGNYNYLYRLFPKPEIYQAGSTPCNGVEGFNCYFPLQSYGFQPTNGVGYQP

SEQ ID NO: 125 - protein, 59 aa, synthetic construct
  REGION 1..59: note = S protein scaffold sequence
SNNLDSKVGGNYNYLYRLFPGTDIYQAGSTPCNGVEGFNCYFPLQSYGFQPTNGVGYQP

SEQ ID NO: 126 - protein, 60 aa, synthetic construct
  REGION 1..60: note = S protein scaffold sequence
SNNLDSKVGGNYNYLYRLFPAHDKIYQAGSTPCNGVEGFNCYFPLQSYGFQPTNGVGYQP

SEQ ID NO: 127 - protein, 87 aa, synthetic construct
  REGION 1..87: note = S protein scaffold sequence
  REGION 27..31: note = MISC_FEATURE - Each residue is any amino acid
  REGION 32..56: note = MISC_FEATURE - Each residue is any amino acid or absent
EEVIAWNSNNLDSKVGGNYNYLYRLFXXXXXXXXXXXXXXXXXXXXXXXXXXXXXXAGFNGYFSLQSYGFQPTNGVGYQPYRVVRRR

SEQ ID NO: 128 - protein, 15 aa, synthetic construct
  REGION 1..15: note = Loop from S protein Scaffold #'s 1, 4, 7, 10, and 23
RKSNLKPFERDISTE

SEQ ID NO: 129 - protein, 32 aa, synthetic construct
  REGION 1..32: note = Loop from S protein Scaffold #'s 5, 8
CKMSECVLGQSKRVQALLFNKVTLAGFNGYFC

SEQ ID NO: 130 - protein, 32 aa, synthetic construct
  REGION 1..32: note = Loop from S protein Scaffold #40
CKMSESVLGQSKRVQALLFNKVTLAGFNGYFC

SEQ ID NO: 131 - protein, 22 aa, synthetic construct
  REGION 1..22: note = Peptide
TFGNPVIPFKDGIYFAATEKSN

SEQ ID NO: 132 - protein, 23 aa, synthetic construct
  REGION 1..23: note = Peptide
TNFRAILTAFSPAQDIWGTSAAA

SEQ ID NO: 133 - protein, 21 aa, synthetic construct
  REGION 1..21: note = Peptide
ISPCSFGGVSVITPGTNASSE

SEQ ID NO: 134 - protein, 21 aa, unidentified
YDPLQPELDSFKEELDKYFKN

SEQ ID NO: 135 - protein, 33 aa, synthetic construct
  REGION 1..33: note = Peptide
LPDPLKPTKRSFIEDLLFNKVTLADAGFMKQYG

SEQ ID NO: 136 - protein, 29 aa, unidentified
ASANLAATKMSECVLGQSKRVDFCGKGYH

SEQ ID NO: 137 - protein, 1270 aa, unidentified
MFVFLVLLPLVSSQCVNLTTRTQLPPAYTNSFTRGVYYPDKVFRSSVLHSTQDLFLPFFSNVTWFHAISGTNGTKRFDNPVLPFNDGVYFASTEKSNIIRGWIFGTTLDSKTQSLLIVNNATNVVIKVCEFQFCNDPFLGVYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTYGVGYQPYRVVVLSFELLHAPATVCGPKKSTNLVKNKCVNFNFNGLTGTGVLTESNKKFLPFQQFGRDIDDTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSHRRARSVASQSIIAYTMSLGAENSVAYSNNSIAIPTNFTISVTTEILPVSMTKTSVDCTMYICGDSTECSNLLLQYGSFCIQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILARLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 138 - protein, 1273 aa, unidentified
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPRRARSVASQSIIAYTMSLGVENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 139 - protein, 1273 aa, unidentified
MFVFLVLLPLVSSQCVNFTNRTQLPSAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNYPFLGVYYHKNNKSWMESEFRVYSSANNCTFEYVSQPFLMDLEGKQGNFKNLSEFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGTIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAAIKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 140 - protein, 805 aa, Homo sapiens [sequence omitted]

SEQ ID NO: 141 - protein, 50 aa, synthetic construct
  REGION 1..50: note = ACE2 scaffold sequence
STIEEQAKTFLDKFNHEAEDLFYQGSGSGNAGDKWSAFLKEQSTLAQMYP

SEQ ID NO: 142 - protein, 54 aa, synthetic construct
  REGION 1..54: note = ACE2 scaffold sequence
STIEEQAKTFLDKFNHEAEDLFYQGSGSGNAGDKWSAFLKEQSTLAQMYPEPEA

SEQ ID NO: 143 - RNA, 25 nt, synthetic construct
  source 1..25: mol_type = other RNA
gctgatgagtacgaacttatgtact

SEQ ID NO: 144 - RNA, 27 nt, synthetic construct
  source 1..27: mol_type = other RNA
agtacataagttcgtactcatcagctt

SEQ ID NO: 145 - RNA, 25 nt, synthetic construct
  source 1..25: mol_type = other RNA
ggaagagacaggtacgttaatagtt

SEQ ID NO: 146 - RNA, 27 nt, synthetic construct
  source 1..27: mol_type = other RNA
aactattaacgtacctgtctcttccga

SEQ ID NO: 147 - RNA, 25 nt, synthetic construct
  source 1..25: mol_type = other RNA
caagctgatgagtacgaacttatgt

SEQ ID NO: 148 - RNA, 27 nt, synthetic construct
  source 1..27: mol_type = other RNA
acataagttcgtactcatcagcttgtg

SEQ ID NO: 149 - protein, 24 aa, Homo sapiens
STIEEQAKTFLDKFNHEAEDLFYQ

SEQ ID NO: 150 - protein, 21 aa, Homo sapiens
NAGDKWSAFLKEQSTLAQMYP

SEQ ID NO: 151 - protein, 66 aa, synthetic construct
  REGION 1..66: note = ACE2 scaffold sequence
STIEEQAKTFLDKFNHEAEDLFYQSSLASWNYNTNITEENVQNMNNAGDKWSAFLKEQSTLAQMYP

SEQ ID NO: 152 - protein, 174 aa, synthetic construct
  REGION 1..174: note = Cyclic peptide
HHHHHHGENLYFKLQAMGMIKIATRKYLGKQNVYDIGVERYHNFALKNGFIASNCAAAAACLSYDTEILTVEYGILPIGKIVEKRIECTVYSVDNNGNIYTQPVAQWHDRGEQEVFEYCLEDGCLIRATKDHKFMTVDGQMMPIDEIFERELDLMRVDNLPNGTAANDENYALA

(19) United States
(12) Patent Application Publication
(10) Pub. No.: US 2023/0242592 A1
(43) Pub. Date: Aug. 3, 2023

(54) IDENTIFICATION OF BIOMIMETIC VIRAL PEPTIDES AND USES THEREOF

(71) Applicant: LIGANDAL, INC., San Francisco, CA (US)

(72) Inventors: Andre WATSON, San Francisco, CA (US); Alexander Izvorski, San Francisco, CA (US)

(21) Appl. No.: 17/822,099

(22) Filed: Aug. 24, 2022

Related U.S. Application Data
(63) Continuation of application No. PCT/US2021/019739, filed on Feb. 25, 2021.
(60) Provisional application No. 62/981,453, filed on Feb. 25, 2020.
(60) Provisional application No. 63/002,249, filed on Mar. 30, 2020.
(60) Provisional application No. 62/706,225, filed on Aug. 5, 2020.
(60) Provisional application No. 63/091,291, filed on Oct. 13, 2020.

(51) Int. Cl. C07K 14/005; C12N 15/62

(52) U.S. Cl. CPC C07K 14/005; C12N 15/62; C12N 2770/20034; C07K 2317/30; C07K 2317/92

(57) ABSTRACT

Disclosed are small peptides derived from the binding interface of each of SARS-CoV-2 spike protein and ACE2 receptor, compositions comprising the same, and prophylactic and therapeutic uses of the peptides and the compositions. Also disclosed is a novel protocol of identifying, designing, and modifying the small peptides based on computer simulation.

### CROSS-REFERENCE TO RELATED APPLICATION(S)

This application is a continuation of International Application No. PCT/US21/019739 filed Feb. 25, 2020, which claims priority to U.S. Provisional Patent Application No. 62/981,453, filed Feb. 25, 2020, U.S. Provisional Patent Application No. 63/002,249, filed Mar. 30, 2020, U.S. Provisional Patent Application No. 62/706,225, filed Aug. 5, 2020, and U.S. Provisional Patent Application No. 63/091,291, filed Oct. 13, 2020, the contents of which are hereby incorporated by reference in their entireties.

### SEQUENCE LISTING

This application contains a Sequence Listing, which was submitted in ASCII format via EFS-Web, and is hereby incorporated by reference in its entirety. The ASCII copy, created on Aug. 24, 2022, is named 2022-08-24 Ligandal 8009.US04 sequence listing and is 160 KB in size.

### BACKGROUND

SARS-CoV-2, which causes COVID-19, is a global pandemic. SARS-CoV-2 and other coronaviruses including MERS and SARS cause severe respiratory illnesses in humans and are believed to have a common origin in viruses that propagate in bats and rodents. Some corona viruses with animal hosts have acquired mutations that extend their host range to include humans. As of March 2020 SARS-CoV-2 has mutated and expanded across the human species; a total of 214 haplotypes (i.e. sequence variations) and 344 different strains have been identified. Most of these variations, gained through mutation, recombination, and natural selection, have been found in the Spike (S) protein. Such variations may lead to even more infective and virulent strains. Exploring the sequence space associated with viral proteins is a difficult problem with critically important implications for evolutionary biology and disease forecasting. While several past studies have attempted to address the problem of viral evolution, few have had access to data sets similar to those compiled for SARS-CoV-2 or to the rich set of novel analytical tools arising from data science, mathematics, and biophysics that are currently available to researchers.

The long-term health consequences of SARS-CoV-2 infection in recovered individuals remain to be seen, however, they include a range of sequelae from neurological to hematological, vascular, immunological, inflammatory, renal, respiratory, and potentially even autoimmune. These long-term effects are particularly concerning when factoring in the known neuropsychiatric effects of SARS-CoV-1, whereby 27.1% of 233 SARS survivors exhibited symptoms meeting diagnostic criteria for chronic fatigue syndrome 4 years after recovery. Furthermore, 40.3% reported chronic fatigue problems and 40% exhibited psychiatric illness. The current preventive approaches include, for example, mRNA vaccine approach and recombinant vaccine approaches comprising virus-like particles, recombinant spike protein fragments, and the like. These vaccine approaches are usually costly, slow to develop, and require live attenuated, recombinant, or mRNA-based approaches that require extensive reengineering to approach novel antigens. While mRNA costs more than $1000/mg to manufacture at lab-bench scale, the peptide approach disclosed herein is a much more cost-effective alternative, at about $5/mg at lab-bench scale.

Rapid and globally scalable vaccine development is of paramount importance for protecting the world from SARS-CoV-2, as well as future lethal disease outbreaks and pandemics. Accordingly, there is an urgent need to better understand the potential variations of genomic sequences of the S protein in SARS-CoV-2 or any other new viruses or the like, and to develop an affordable, globally deployable, room temperature stable, and repeatedly administrable therapeutic with low risk of complications across the general population.

### SUMMARY

In one aspect, disclosed herein is a scaffold comprising a truncated peptide fragment from the binding domain of SARS-CoV-2 spike (S) protein or ACE2 receptor, wherein the scaffold substantially maintains the structure, conformation, and/or binding affinity of the native protein. In certain embodiments, the scaffold has a size of between 40 and 200 amino acid residues. In certain embodiments, the scaffold comprises two critical binding motifs from the CoV-2 spike protein binding interface. In certain embodiments, the scaffold comprises two critical binding motifs from the ACE2 binding interface. In certain embodiments, the two critical binding motifs are connected by a linker such as a GS linker. In certain embodiments, the linker has a size of between 1 and 20 amino acid residues. In certain embodiments, the scaffold comprises one or more modifications including an insertion, a deletion, and/or a substitution. In certain embodiments, the scaffold further comprises one or more immuno-epitopes, one or more tags, one or more conjugatable domains, and/or a polar head or tail. In certain embodiments, one or more scaffolds are connected via one or more linkers to form a multi-valent scaffold. In certain embodiments, one or more scaffolds are attached to an immune-response eliciting domain such as an Fc domain (e.g., a human Fc domain or a humanized Fc domain) to form a fusion protein. In certain embodiments, one or more scaffolds are attached to a substrate such as a nanoparticle or a chip. In certain embodiments, one or more scaffolds are conjugated to another peptide or therapeutic agent.

In another aspect, disclosed herein is a composition comprising one or more scaffolds, one or more conjugates, or one or more fusion proteins disclosed herein. In certain embodiments, the composition further comprises one or more pharmaceutically acceptable carriers, excipients, or diluents. In certain embodiments, the composition is formulated into an injectable, inhalable, oral, nasal, topical, transdermal, uterine, or rectal dosage form. In certain embodiments, the composition is administered to a subject by a parenteral, oral, pulmonary, buccal, nasal, transdermal, rectal, or ocular route. In certain embodiments, the composition is a vaccine composition.

In another aspect, disclosed herein is a method of treating or preventing SAR-CoV-2 infection in a subject comprising administering to the subject a therapeutically effective amount of one or more scaffolds, one or more conjugates, one or more fusion proteins, or a composition comprising the one or more scaffolds, one or more conjugates, or one or more fusion proteins disclosed herein. In certain embodiments, the subject is a mammal. In certain embodiments, the subject is human.

In another aspect, disclosed herein is a method of blocking SAR-CoV-2 virus entry in a subject comprising administering to the subject a therapeutically effective amount of one or more scaffolds, one or more conjugates, one or more fusion proteins, or a composition comprising the one or more scaffolds, one or more conjugates, or one or more fusion proteins disclosed herein. In certain embodiments, the subject is a mammal. In certain embodiments, the subject is human.

In another aspect, disclosed herein is a method of targeted delivery of one or more therapeutic agents comprising conjugating the one or more therapeutic agents to one or more scaffolds disclosed herein, and delivering the conjugate to a subject in need thereof.

In another aspect, disclosed herein is a method of obtaining a scaffold that mimics the binding of the native protein from which the scaffold is derived. The method entails the steps of producing a three-dimensional binding model of a first binding partner and a second binding partner, determining the binding interface on each binding partner based on the binding model, analyzing the binding interface to preserve the structure and/or conformation of each binding partner in its native, free or bound state, determining the critical binding residues based on thermodynamic calculation (ΔG), and determining the amino acid sequence of the binding interface of each binding partner to obtain the scaffold. In certain embodiments, the three-dimensional binding is produced by a computer program such as SWISS-MODEL. In certain embodiments, the three-dimensional binding is based on homology of either the first binding partner or the second binding partner to a protein of known sequence and/or structure. In certain embodiments, the method further entails designing scaffolds of various conformations or folding states to fit with the corresponding binding partner.

### BRIEF DESCRIPTION OF THE DRAWINGS

This application contains at least one drawing executed in color. Copies of this application with color drawing(s) will be provided by the Office upon request and payment of the necessary fees.

FIG. 1 shows the crystal structure of SARS-CoV-1 (PDBID 6CS2) bound to ACE2 (left) compared to simulated structure of SARS-CoV-2 bound to ACE2 (right). Amino acid residues contributing positively to binding (−ΔG) are shown in green, amino acid residues having about 0 ΔG are shown in yellow and repulsory amino acid residues (+ΔG) are shown in pink (left) or in orange (right).

FIG. 2A shows the 3D structure of two previously published SARS-CoV-1 immuno-epitopes. FIGS. 2B-2D show the 3D structure and the locations of the deduced CoV-2 immuno-epitopes based on homology to SARS-CoV-1.

FIG. 3 shows the MHC-I binding prediction results of immuno-epitopes. “ImmunoEpitope1”=SEQ ID NO:67; “ImmunoEpitope2”=SEQ ID NO:69; KMSECVLGQSKRV=SEQ ID NO:71; LLFNKVTLA=SEQ ID NO:7; SFIEDLLFNKV=SEQ ID NO:68.

FIG. 4 shows the position of CoV-2 S protein antibody epitopes identified by others in the CoV-2 S protein (residues 15-1137 of SEQ ID NO:2 pictured). The CoV-2 scaffold in the wildtype protein is double underlined. The epitopes are shown in bold while the epitopes having high antigenicity scores are shown in bold and underlined.

FIG. 5 shows the truncated CoV-2 S protein aligned to ACE2 and the locations of the antibody epitopes (magenta) and the ACE2 binding residues (green).

FIG. 6 depicts three-dimensional molecular modeling of three representative linkers in the bound conformation. The backbone is depicted as a blue coil. Side chain atoms are color coded in PyMol using the command color>by chain>chainbows and Color>by>element>HNOS where H=white, N=blue, O=red, and S=yellow. Representative sequences depicted are

(SEQ ID NO: 116)
SNNLDSKVGGNYNYLYRLFDGTEIYQAGSTPCNGVEGFNCYFPLQSYGFQ
PTNGVGYQP;
(SEQ ID NO: 119)
SNNLDSKVGGNYNYLYRLFNANDKIYQAGSTPCNGVEGFNCYFPLQSYGF
QPTNGVGYQP;
(SEQ ID NO: 122)
SNNLDSKVGGNYNYLYRLFPGTEIYQAGSTPCNGVEGFNCYFPLQSYGFQ
PTNGVGYQP.

FIG. 7A illustrates the binding of Scaffold #15 (SEQ ID NO:86) to residues 19-169 of ACE2 (SEQ ID NO:140). The B cell epitopes are shown in magenta, the T cell epitopes are shown in orange, and the ACE2 binding sites are shown in green. FIG. 7B illustrates that a modified CoV-2 scaffold having 59 amino acids (with 18 amino acids eliminated from the wildtype sequence) preserves the binding affinity to ACE2. FIG. 7C illustrates that a modified CoV-2 scaffold having 67 amino acids (with 10 amino acids eliminated from the wildtype sequence) preserves the binding affinity to ACE2. The B cell antibody immuno-epitopic regions are shown in magenta, the T cell receptor binding, MHC-1 and MHC-2 loading regions are shown in orange, and the ACE2 binding regions are shown in green.

FIGS. 8A-8B show that S protein Scaffold #9 (SEQ ID NO:80) can be fitted to ACE2 (FIG. 8A; residues 19-107 of ACE2 shown) to determine its ACE2 binding affinity and K_{D} prediction based on computer modeling (FIG. 8B).

FIG. 9A shows the computer modeling of CoV-1 (cyan) and CoV-2 (navy) bound to ACE2 (red) based on homology of CoV-1 and CoV-2. FIG. 9B shows the computer modeling of CoV-1 (cyan) bound to ACE2 (red). FIG. 9C shows the computer modeling of CoV-2 (navy) bound to ACE2 (red).

FIGS. 10A and 10B show the ΔG calculation to determine key binding residues for CoV-2 and CoV-1, respectively.

FIGS. 11A-11B show the thermodynamic modeling of CoV-2 bound to ACE2, with the binding interface enlarged in FIG. 11B. FIG. 11C shows two critical binding motifs determined for CoV-2: residues 437-455 (SEQ ID NO:65) and residues 473 to 507 (SEQ ID NO:66. The amino acid residues having a negative ΔG, a positive ΔG, and about 0 ΔG are shown in green, orange, and yellow, respectively. The backbone residues are shown in navy. L455 and P491 are shown in magenta.

FIGS. 12A-12J illustrate the folding possibilities (center0 through center9 conformation shown in PyMOL) for CoV-2 Scaffold #1 having an amino acid sequence of SEQ ID NO:72.

FIG. 13A shows the binding of center0 of CoV-2 Scaffold #9 (SEQ ID NO:80) with ACE2. FIG. 13B shows the binding of center0 and center9 of CoV-2 Scaffold #9 with ACE2. FIGS. 13C-13D show chaotic assortment of center0-center9 of CoV-2 Scaffold #9 with ACE2, showing reasonable average folding and locations of all possible folding states given Heisenberg Uncertainty Principle. FIG. 13D shows the enlarged binding interface of Scaffold #9 and ACE2.

FIG. 14A depicts a simulation of ACE2 bound to CoV-2 S protein. FIGS. 14B-14D depict ACE2 Scaffold 1 (SEQ ID NO:141) (purple), simulated via RaptorX, overlaid with wildtype hACE2 (red). The critical binding residues of ACE2 at the interface with the CoV-2 S protein are highlighted green.

FIG. 15A shows computer modeling of ACE2 Scaffold 1 (SEQ ID NO:141 truncated from the ACE2 protein. FIG. 15B depicts the molecular modeling of ACE2 Scaffold 1 (purple, with critical binding residues shown in green) with the CoV-2 S protein (blue, with antibody binding domains shown in teal arrow). The scaffold binds to the CoV-2 S protein while preserving the presentation of antibody-binding immuno-epitopic regions of the S protein while bound. FIG. 15C depicts ACE2 Scaffold 1 bound to the CoV-2 S protein. ACE2 Scaffold 1 is not predicted to affect the immune binding domains (pink) of the CoV-2 S protein.

FIG. 16A shows the binding to ACE2 by the Cryo-EM structure of CoV-2 S protein published by others, and FIG. 16B shows the binding to ACE2 by CoV-2 S protein based on SWISS-MODEL.

FIG. 17A shows the simulated conformation with ACE2 using the structure published by others (top) and the computer simulated structure of this disclosure (bottom).

FIG. 17B shows the comparison of the Cryo-EM structure of CoV-2 published by others (left) to the disclosed truncated and labeled SWISS-MODEL simulated structure (right). The red dotted oval indicates the location of the missing residues from the Cryo-EM structure. Purple regions indicate B cell immuno-epitopes determined by others, while orange regions indicate ACE2-repulsory regions, green regions indicate ACE2-binding regions, and yellow regions indicate ACE2-neutral regions as determined via PDBePISA.

FIG. 18 shows that a custom-built peptide robot completed synthesis of a 9-amino acid MHC-1 loading epitope in about 24 minutes, allowing for rapid prototyping prior to commercial scale-up.

FIG. 19A shows head-to-tail cyclization of the side chain protected peptide in solution by amide coupling using Scaffold #47 (“Ligandal-05,” SEQ ID NO:118) as an example. FIG. 19B shows on resin head-to-tail cyclization by amide coupling using Scaffold #48 (“Ligandal-06,” SEQ ID NO:119) as an example. FIG. 19C shows cyclization of purified linear thioester peptide by NCL using Scaffold #46 (“Ligandal-04,” SEQ ID NO:117) as an example.

FIGS. 20A-20I are charts depicting biolayer interferometry of Scaffold #4 (“Peptide 1,” SEQ ID NO:75), Scaffold #7 (“Peptide 4,” SEQ ID NO:78), Scaffold #8 (“Peptide 5,” SEQ ID NO:79), and Scaffold #9 (“Peptide 6,” SEQ ID NO:80) associated with ACE2-biotin captured on streptavidin sensor tips (2.5 nm capture) to determine dissociation constant of the scaffolds to ACE2. All scaffolds exhibited potent inhibition of RBD binding to ACE2 at 10 μM concentrations. As shown in FIGS. 20A-20D, a clear binding to ACE2 was observed for each scaffold with increasing concentrations (blank values were subtracted). As shown in FIGS. 20E-20H, a dose-response curve was also observed, whereby RBD was able to strongly associate with each sensor at 35 μM in the absence of peptide (green, top curve), and experienced a peptide-dose-response-dependent inhibition of binding (blue, cyan and red represent 10, 3 and 1 μM concentrations, respectively). FIG. 20I corresponds to RBD-biotin captured on streptavidin sensor tips (5 nm capture), and subsequently bound to ACE2.

FIGS. 21A-21F are charts depicting biolayer interferometry of the scaffolds associated with a neutralizing antibody captured on anti-human IgG (AHC) sensor tips (1 nm capture) was used to determine dissociation constant of Scaffold #4 (“Peptide 1,” SEQ ID NO:75), Scaffold #7 (“Peptide 4,” SEQ ID NO:78), Scaffold #8 (“Peptide 5,” SEQ ID NO:79), and Scaffold #9 (“Peptide 6,” SEQ ID NO:80) to the neutralizing antibody (FIGS. 21A-21D). The dissociation constant of increasing concentrations of RBD was determined with anti-RBD neutralizing antibody (FIG. 21E). FIG. 21F shows that 117 nM RBD was mixed with increasing concentrations of ACE2 prior to introduction to neutralizing antibodies bound to the sensors to demonstrate ACE2's inhibition of neutralizing antibody binding to the RBD.

FIG. 22 shows luminescence (RLU) of ACE2-HEK293 cells following SARS-CoV-2 spike infection at 60 hours post-infection when co-transfected with Scaffold #4 (“Peptide 1,” SEQ ID NO:75), Scaffold #7 (“Peptide 4,” SEQ ID NO:78), Scaffold #8 (“Peptide 5,” SEQ ID NO:79), and Scaffold #9 (“Peptide 6,” SEQ ID NO:80). Control groups included untransfected ACE2-HEK293 cells (no virus) and ACE2-HEK293 cells transfected with the SARS-CoV2 spike protein.

FIGS. 23A-23D show luminescence (RLU) in ACE2-HEK293 cells transfected with the SARS-CoV-2 spike protein or no virus (control) and Scaffold #8 (“Peptide 5,” SEQ ID NO:79) (FIG. 23A), soluble ACE2 (FIG. 23B), soluble receptor-binding domain (RBD) of the SARS-CoV-2 spike protein (FIG. 23C), and a SARS-CoV-2 neutralizing antibody (neuAb) (FIG. 23D).

FIG. 24A shows that Scaffold #4 (“LGDL_NIH_001,” SEQ ID NO:75), Scaffold #7 (“LGDL_NIH_004,” SEQ ID NO:78), Scaffold #8 (“LGDL_NIH_005,” SEQ ID NO:79), and Scaffold #9 (“LGDL_NIH_006,” SEQ ID NO:80) exhibited over 90% inhibition of viral load (EC90) in live virus at micromolar concentrations. FIG. 24B shows that the scaffolds tested in FIG. 24A were not toxic at the effective concentrations.

FIG. 25 depicts three-dimensional molecular modeling of Scaffold #4 (“Peptide 1,” SEQ ID NO:75 based on a 180 ns run (single trajectory) in OpenMM starting from the native-like conformation.

FIG. 26 is a chart plotting Rosetta score (REU) of Scaffold #4 (SEQ ID NO:75 at indicated timepoints.

FIGS. 27A and 27B are diagrams of epitopes on the S protein that are only exposed during fusion.

FIGS. 28A and 28B are diagrams of binding sites which would prevent the process from moving to the next step of neutralizing. FIG. 28C shows the enlarged binding site.

FIGS. 29A and 29B (enlarged) depict three-dimensional molecular modeling of the sequence KMSECVLGQSKRV (SEQ ID NO:8) (shown in red) fitted to the SARS-CoV-2 spike protein (green). SEQ ID NO:8 corresponds to one of the binding sites identified in FIGS. 27-28 located in the hinge between heptad repeat (HR) 1 (HR1) and HR2 during the pre-bundle stage.

FIG. 30 depicts a diagram for extein insertion placement.

FIGS. 31A-31D are diagrams generated during peptide screening and optimization.

FIGS. 32A-32B show sequence alignments of representative SARS-CoV-2 S protein scaffolds disclosed herein. Alignment shows amino acid residues 433-511 of SEQ ID NO:2. Critical binding motifs are underlined. Substitutions are double underlined and highlighted yellow. GS linkers are bolded and highlighted blue. Epitopes for B cell and T cell binding are bolded, italicized, and highlighted green. EPEA C-tags are italicized and highlighted gray. Poly charged N- and C-terminal residues are squiggly underlined and highlighted pink. Alternative TCR epitopes are highlighted red.

FIGS. 33A-33E illustrate the siRNA designing process using the IDT siRNA design tool, including the locations and sequences of the selected sense and anti-sense strands (SEQ ID NOs:143-148).

FIG. 34 depicts a three-dimensional simulation model of SARS-CoV-1 bound to angiotensin-converting enzyme 2 (ACE2) (PDB ID 6CS2; red) to approximate the binding interface of the SWISS-MODEL simulated SARS-CoV-2 (left); and selected MHC-I and MHC-II epitope regions for inclusion in Scaffold #8) (pink) represent P807-K835 and A1020-Y1047 in the 51 spike protein. The model on the right depicts the receptor-binding domain (RBD) of the SARS-CoV-2 spike protein (blue/multi-colored) simulated binding with ACE2 (red). The simulation model identifies predicted thermodynamically favorable (green), neutral (yellow), and unfavorable (orange) interactions. Outer bounds of amino acids used to generate the scaffold (V433-V511) are shown in cyan on the right.

FIG. 35 depicts a three-dimensional simulation model of the ACE2 receptor (red) aligned with Scaffold #4, #7, #8, and #9 (top, from left to right). Multiple folding states for Peptide 5 are shown in simulated binding to ACE2 (bottom). Predicted binding residues are indicated in green (top and bottom).

FIG. 36 shows SARS-CoV-2 genomic sequence (SEQ ID NO: 1). Nucleotides 21536-25357 (underlined) encode S protein of SEQ ID NO:2. Nucleotides 26218-26445 (double underlined) encode envelope protein of SEQ ID NO:3.

FIG. 37 shows the amino acid sequence of SARS-CoV-2 spike (S) protein (SEQ ID NO: 2).

FIG. 38 shows the amino acid sequence of ACE2 (SEQ ID NO: 140).

### DETAILED DESCRIPTION

As disclosed herein, by combining methods from mathematical data science, biophysics, and experimental biology, the sequences of the S protein that are most likely to expand the host range and increase the stability of SARS-CoV-2 in the human population through natural selection can be predicted. A computational pipeline is developed to estimate the mutation landscape of the SARS-CoV-2 S protein. The predicted sequences are experimentally engineered and their binding to the human receptor ACE2 is measured using biochemical assays and cryo-electron microscopy.

Novel mathematical approaches, inspired by the structure of genetic algorithms, are developed for the identification of highly probable sequences of the SARS-CoV-2's S-protein. More specifically, the disclosed approach incorporates descriptors from graph theory, topological data analysis, and computational biophysics into a new machine learning framework that combines neural networks and genetic algorithms. This powerful interdisciplinary approach allows the use of existing data from SARS-CoV-2 to uncover a few candidate sequences that are most likely to occur in the evolution of its viral S-protein. These results are experimentally validated by generating peptides from the obtained sequences. The resulting pipeline provides new solution to better understand the mutation landscape of viral proteins.

As disclosed herein, in silico analysis was conducted to generate and screen novel peptides (“scaffolds”) designed to serve as competitive inhibitors to the SARS-CoV-2 spike (S) protein by predicting 1) ACE2 receptor binding regions, 2) immuno-epitopic regions for T cell receptor MHC-I and MHC-II loading, and 3) immuno-epitopic regions for B cell receptor or antibody binding. As demonstrated in the working examples, three-dimensional modeling and in silico analysis were used to examine predicted structures of the novel peptides, various sequence modifications were evaluated (e.g., by examining Rosetta energy unit (REU) scores for candidate peptides), and predicted binding models were simulated by computer. Based on these results, provided herein are methods for generating and optimizing peptide scaffolds for use as competitive inhibitors in vaccine development by taking a peptide sequence (e.g., the SARS-CoV-2 spike protein), introducing sequence modifications, and using three-dimensional modeling techniques to predict folding or binding conformation. Also provided herein are optimized peptide scaffolds designed using these methods, formulations comprising these peptide scaffolds, and methods of using these peptide scaffolds and formulations to competitively inhibit viral proteins or treat viral infection, and the use of these peptide scaffolds and formulations as vaccines to prevent viral infection.

Accordingly, this disclosure relates to a breakthrough approach for rapid vaccine prototyping. In some aspects, the disclosed vaccine approach provides a fully synthetic scaffold for mimicking T-cell receptor and antibody binding epitopes, which can be rapidly custom-tailored to new mutant forms of a virus. Additionally, the synthetic scaffold can serve as a targeting ligand mimicking viral entry to target diseased cells and tissues with therapeutic agents. These “mini viral” scaffolds can be synthesized in hours, and rapidly scaled to a scale of over 100 kg to meet global needs. Additionally, scaffolds provided herein may separately be used in place of small molecules for inhibiting binding cleft interactions.

The scaffolds disclosed herein are peptides generated by modeling off the SARS-CoV-2 spike protein receptor binding motif (RBM) conserved motifs, and have the potential utility as a prophylactic, immune-stimulant, and therapeutic agent against the virus. Therefore, also disclosed herein are compositions comprising one or more scaffolds, which can be used for: 1) inhibiting ACE2-spike interaction and viral entry into ACE2-expressing cells, 2) promoting binding to neutralizing antibodies without competitively displacing neutralizing antibody binding to the RBD; and/or 3) preventing soluble ACE2 association with the RBD.

Detailed in this disclosure are the simulation, design, synthesis and characterization of peptide scaffolds designed to block viral binding to cells expressing ACE2, while also stimulating an immune response and promoting exposure of the spike protein for recognition by the immune system. In contrast to neutralizing antibody therapies and other approaches that seek to target the virus, a biomimetic virus decoy peptide technology is developed to compete for binding with cells and expose the virus for binding to neutralizing antibodies.

### I. Computer-Assisted 3D Modeling

A. Analyzing the Binding Interface

In one aspect, this disclosure relates to methods of computer-assisted three-dimensional (3D) modeling to investigate protein-protein interactions. These methods entail producing a 3D model of a first binding partner and a second binding partner, determining the amino acid sequence, the 3D structure, and the conformation of the interface of each binding partner, truncating the binding interface of each binding partner while maintaining the 3D structure of each to obtain a scaffold representing each binding partner, determining the binding affinity of each amino acid residue in the scaffold based on calculation of thermodynamic energy of each residue, and determining the location and sequence of critical binding motifs in the scaffold. In certain embodiments, the 3D model is produced with SWISS-MODEL based on protein sequence homology to the first binding partner or the second binding partner. Various modifications can be made to the scaffold to maintain or improve the structure, conformation, and binding affinity of the scaffold. These modifications include but are not limited to insertions, deletions, or substitutions of one or more amino acid residues in the scaffold. As detailed in this disclosure, various linkers, conjugatable domains, and/or immuno-epitopes can be added to the scaffold to obtain multi-functional scaffolds. In certain embodiments, one or more amino acids that are not critical for binding can be deleted or substituted. In certain embodiments, the binding partners are SARS-CoV-2 S protein and ACE2. In certain embodiments the S protein has the amino acid sequence set forth in SEQ ID NO:2. In other embodiments, the S protein is a variant, including but not limited to B.1.1.7 variant (SEQ ID NO:137), B.1.351 variant (SEQ ID NO:138), or P.1 variant (SEQ ID NO:139). Other variants of coronavirus can be found at nextstrain.org/ncov/global. In certain embodiments ACE2 has the amino acid sequence set forth in SEQ ID NO:140.

As used herein, the term “scaffold” means a continuous stretch of amino acid sequence located at the binding interface of a binding partner and involved with binding to the other binding partner. In certain embodiment, the scaffold has a size of less than about 120 amino acid residues, less than about 110 amino acid residues, less than about 100 amino acid residues, less than about 90 amino acid residues, less than about 80 amino acid residues, less than about 70 amino acid residues, less than about 60 amino acid residues, or less than 50 amino acid residues. In certain embodiments, the scaffold maintains the 3D structure and/or conformation of the native, free, or bound state of the protein from which its binding sequence(s) is derived. For example, the scaffold may be designed to maintain an α-helix and/or β sheet structure when truncated from a wildtype protein sequence. In certain embodiments, the scaffold may comprise one or more modifications such as insertions, deletions, and/or substitutions, provided those modifications do not substantially decrease, and in some embodiments actually increase, binding affinity of the scaffold to its binding partner.

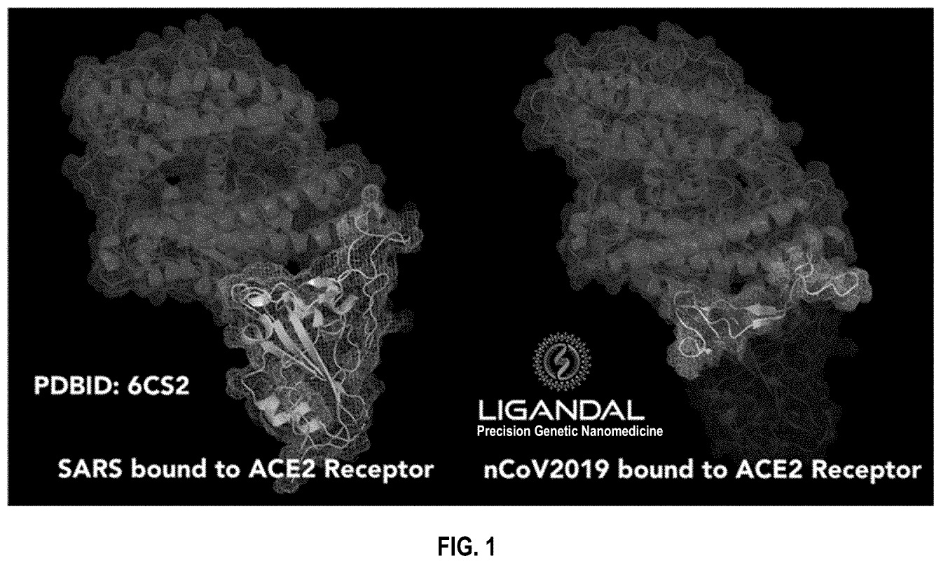

As disclosed herein, the protein sequence of the SARS-CoV-2 spike protein (SARS-CoV-2 or CoV-2; SEQ ID NO:2) was compared to SARS coronavirus protein sequence (PDB ID 6CS2) to produce a 3D model of CoV-2 binding to ACE2 (FIG. 1). The binding interface of each of CoV-2 and ACE2 was investigated to determine a stretch of amino acid residues involved in binding. This stretch of amino acid sequence may be truncated from the remaining protein sequence, and the structure and/or conformation of this stretch of amino acid sequence is maintained to simulate that of the native protein in free or bound state, thereby to obtain the CoV-2 scaffold or the ACE2 scaffold of this disclosure.

Accordingly, disclosed herein is a CoV-2 scaffold, which has an amino acid sequence at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or 100% identical to the amino acid sequence of residues 433-511 of SEQ ID NO:2

(VIAWNSNNLDSKVGGNYNYLYRLFRKSNLKPFERDISTEIYQAGSTPCN
GVEGFNCYFPLQSYGFQPTNGVGYQPYRVV).

In some embodiments, the CoV-2 scaffold has an amino acid sequence at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or 100% identical to the amino acid sequence:

(“Scaffold #1; SEQ ID NO: 72)
VIAWNSNNLDSKVGGNYNYLYRLFRKSNLKPFERDISTEIYQAGSTPCNG
VEGFNCYFPLQSYGFQPTNGVGYQPYRVV.

In SEQ ID NO: 72 above, amino acid residues in the CoV-2 S protein backbone are shown in plain letters (including 433V-436W, F456-I472, and Y508-V511), amino acid residues having a ΔG of about 0, which are neutral in binding, are underlined (including N437, S438, N440-K444, G447, N448, N450-L452, R454, S477-V483, G485, C488, P491, L492, F497, G504, and P507), amino acid residues having a negative ΔG, which are critical binding residues, are shown in bold (including N439, Y449, Y453, Q474, E484, N487, Q493-Y495, Q498, P499, N501, and Q506), and amino acid residues having a positive ΔG, which are repulsory residues, are shown in italicized (including V445, G446, L455, Y473, A475, G476, F486, Y489, F490, G496, T500, G502, V503, and Y505).

Based on the computer modeling and the calculation of thermodynamic energy, it is determined that one CoV-2 scaffold of this disclosure comprises a first critical binding motif comprising residues 437 to 455 of SEQ ID NO: 2, a second critical binding motif comprising residues 473 to 507 of SEQ ID NO: 2, and a backbone region comprising residues 456 to 472 of SEQ ID NO: 2. The first and second critical binding motif directly interact with ACE2 on the binding interface, while the backbone region comprises amino acid residues that do not directly interact with ACE2.

The CoV-2 scaffold may further comprise one or more amino acids from the CoV-2 S protein backbone at the N-terminus, the C-terminus, or both, to achieve a desired size. In some embodiments, the CoV-2 scaffold comprises from about 40 to about 200 amino acid residues, from about 50 to about 100 amino acid residues, from about 55 to about 95 amino acid residues, from about 60 to about 90 amino acid residues, from about 65 to about 85 amino acid residues, from about 70 to about 80 amino acid residues. In some embodiments, the CoV-2 scaffold comprises about 50 amino acid residues, about 55 amino acid residues, about 60 amino acid residues, about 65 amino acid residues, about 70 amino acid residues, about 75 amino acid residues, about 80 amino acid residues, about 85 amino acid residues, about 90 amino acid residues, about 95 amino acid residues, or about 100 amino acid residues.

Although the size of the CoV-2 scaffold can vary, and certain modifications such as insertions, deletions, and/or substitutions can be incorporated, the scaffold maintains the structure and/or conformation of its native state with regard to the ACE2 binding interface. Preservation of this structure and/or conformation allows the scaffold to bind ACE2 with the same or greater affinity than the full-length S protein despite its truncation. For example, the β sheet structure is maintained and can be stabilized by further modifications. In some embodiments, the CoV-2 scaffold comprises L455C and P491C substitutions such that a disulfide bond is formed between location 455 and location 491 to stabilize the β sheet structure. These two locations appear to be in proximity to each other in the native CoV-2 S protein bound to ACE2 based on computer modeling. In some embodiments, the CoV-2 scaffold comprises one or more mutations to replace one or more of the existing Cys residues such that the only Cys residues remaining are the ones introduced at locations 455 and 491 to avoid any undesirable interference of formation of a disulfide bond. For example, Cys can be substituted with Gly, Ser, or any other residue as long as the substitution does not compromise the binding affinity to ACE2. Some examples of replacing Cys residues include but not limited to C480G, and C488G.

In certain embodiments, the CoV-2 scaffold disclosed herein may further comprise a loop to connect the N-terminal residue and the C-terminal residue using a linker such as an amine-carboxy linker to obtain a head-to-tail cyclized scaffold. In certain embodiments, cyclization of the scaffold provides increased stability with lower free energy, enhanced folding, binding, or conjugation to a substrate, and/or enhanced solubility. The loop does not directly interact with the scaffold's binding partner. In certain embodiments, the loop allows the scaffold to be attached to an siRNA payload or other substrates. In certain embodiments, the loop comprises 1-200 amino acid residues. In certain embodiments, the loop comprises less than about 150 amino acid residues. Depending on the desired conformation of the scaffold, linkers, conjugatable domains, a polar head or tail, etc., one can adjust the size of the loop accordingly. In some embodiments, the loop comprises 9-15 Arg and/or Lys residues. In some embodiments, the loop comprises a conjugatable domain such as maleimide or other linkers to conjugate the scaffold to a substrate or a poly amino acid chain. In some embodiments, the loop comprises one or more immune-activating poly amino acid chain or immune-reactive glycan. The N-terminus and C-terminus can also be connected by forming a disulfide bond, any other appropriate linker (flexible or rigid), click chemistry, PEG, polysarcosine, or bioconjugated. Thus, the peptides may be cyclized, stabilized, linear, otherwise click-chemistry or bioconjugated, or substituted with non-natural amino acids, peptoids, glycopeptides, lipids, cholesterol moieties, polysaccharides, or anything that enhances folding, binding, solubility, or stability.

Also disclosed herein is an ACE2 scaffold, which is at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or 100% identical to the amino acid sequence of residues 19-84 of SEQ ID NO:140.

In some embodiments, the ACE2 scaffold is at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or 100% identical to the amino acid sequence:

(SEQ ID NO: 151)
STIEEQAKTFLDKFNHEAEDLFYQSSLASWNYNTNITEENVQNMNNAGDK
WSAFLKEQSTLAQMYP

In SEQ ID NO:151 above, the amino acid residues having a negative ΔG, which are critical binding residues, are shown in bold (including S19, Q24, D38, Q42, E75, Q76, and Y83).

Based on the computer modeling and the calculation of thermodynamic energy, it is determined that an ACE2 scaffold of this disclosure comprises a first critical binding motif comprising amino acid residues 19 to 42 of SEQ ID NO:140, a second critical binding motif comprising residues 64 to 84 of SEQ ID NO:140, and a backbone region comprising residues 43 to 63 of SEQ ID NO:140. The first and second critical binding motif directly interact with CoV-2 S protein on the binding interface, while the backbone comprises amino acid residues on the backbone of ACE2 and does not directly interact with CoV-2 S protein.

In some embodiments, the ACE2 scaffold comprises a linker (shown in bold) connecting the two critical binding motifs, see for example, ACE2 Scaffold 1 (SEQ ID NO:141):

STIEEQAKTFLDKFNHEAEDLFYQGSGSGNAGDKWSAFLKEQSTLAQMYP

In some embodiments, the ACE2 scaffold further comprises an EPEA C-tag (underlined), see for example, ACE Scaffold 2 (SEQ ID NO:142):

STIEEQAKTFLDKFNHEAEDLFYQGSGSGNAGDKWSAFLKEQSTLAQMYP
EPEA

The ACE2 scaffold may further comprise one or more amino acids or monomeric units from the ACE2 protein backbone or recreating the binding effect of ACE2 at the appropriate interface with the spike protein as derived from ACE2's N-terminus, the C-terminus, or both, to achieve a desired size, folding and affinity. In some embodiments, the ACE2 scaffold comprises from about 10 to about 200 amino acid residues, from about 50 to about 100 amino acid residues, from about 55 to about 95 amino acid residues, from about 60 to about 90 amino acid residues, from about 65 to about 85 amino acid residues, from about 70 to about 80 amino acid residues. In some embodiments, the ACE2 scaffold comprises about 50 amino acid residues, about 55 amino acid residues, about 60 amino acid residues, about 65 amino acid residues, about 70 amino acid residues, about 75 amino acid residues, about 80 amino acid residues, about 85 amino acid residues, about 90 amino acid residues, about 95 amino acid residues, or about 100 amino acid residues.

Although the size of the ACE2 scaffold can vary, and certain modifications such as insertions, deletions, and/or substitutions can be incorporated, the scaffold maintains the structure and/or conformation of its native state with regard to the CoV-2 S protein binding interface. Preservation of this structure and/or conformation allows the scaffold to bind the S protein with the same or greater affinity than the full-length ACE2 protein despite its truncation.

In certain embodiments, the N-terminus, C-terminus, or both termini of the ACE2 scaffold are modified with any number of bioconjugation motifs, linkers, spacers, tags (such as his-tag and C-tag) etc. In certain embodiments, one or more amino acids that are not critical for binding to CoV-2 S protein are deleted or substituted.

Additional scaffolds can be designed to mimic ACE2 binding to the CoV-2 S protein. These ACE2 scaffolds can bind to the CoV-2 virus to coat the virus such that the virus is unable to bind to ACE2 thereby to inhibit viral entry into human (or other hosts) body. Moreover, an ACE2 scaffold can be further modified to include, for example, a fragment crystallizable (Fc) domain or an alternate domain that serves to activate an immune response.

ACE2 Scaffold 1 (SEQ ID NO:141), comprising a first critical binding motif, a second critical binding motif, and a linker connecting the critical binding motifs, is predicted to have a higher affinity for the CoV-2 S protein than wildtype ACE2. Additionally, in contrast to ACE2, which binds to CoV-2 S protein and blocks the immuno-epitopic region of CoV-2 S protein, ACE2 Scaffold 1 is not expected to affect the immune binding domains of the CoV-2 S protein and allows the immune system to identify the CoV-2 virus. Similar to other scaffolds provided herein, ACE2 Scaffold 1 may be provided in a nanoparticle or other suitable substrate and may act to aggregate the virus. For instance, the N- or C-termini may be modified with any number of bioconjugation motifs, linkers, spacers, and the like; and may have various substrates including buckyballs (e.g., C60/C70 fullerenes), branched PEGs, hyper-branched dendrimers, single-walled carbon nanotubes, double-walled carbon nanotubes, KLH, OVA, and/or BSA. ACE2 Scaffold 1 is predicted to have a higher affinity for the virus spike proteins than free ACE2.

B. Analyzing the Immune-Epitopes

Immune Epitope Database (IEDB) was utilized to predict key epitopes prior to clinical data emerging on various T cell receptor (TCR) responses across populations with various HLA alleles. These predicted epitopes were compared to known epitopes for MHC-I and MHC-II response in SARS-CoV-1. It was previously reported that S5 peptide having an amino acid sequence of LPDPLKPTKRSFIEDLLFNKVTLADAGFMKQYG (SEQ ID NO:135) (residues 788-820 of SARS-CoV-1) and S6 peptide having an amino acid sequence of ASANLAATKMSECVLGQSKRVDFCGKGYH (SEQ ID NO:136) (residues 1002-1030 of SARS-CoV-1) exhibited immunogenic responses similar to those found in a parallel investigation using truncated recombinant protein analogs of the SARS-CoV S protein (2). The S5 peptide was defined based on known immunogenicity of the monovalent peptide in terms of its ability to illicit an MHC-I response and antibody response, whereas many other peptides were only immunogenic while present multivalently. The S6 peptide represents a known MHC-II domain from SARS-CoV-1.

These immuno-epitopes of SARS-CoV-1 S protein were aligned to CoV-2 S protein to determine likely immunogenic sites on CoV-2 S protein. Based on homology, the corresponding immuno-epitopes in CoV-2 S protein are identified as follows, and are also designed to overlap with the regions of the S2 spike in its pre-fusion conformation following TMPRSS2 cleavage of the S1-S2 interface:

(SEQ ID NO: 67)
(QIL)PDPSKPSKRSFIEDLLFNKVTLADAGFIK
(locations 804-835),
and
(SEQ ID NO: 69)
ASANLAATKMSECVLGQSKRVDFCGKGY
(locations 1020-1047)

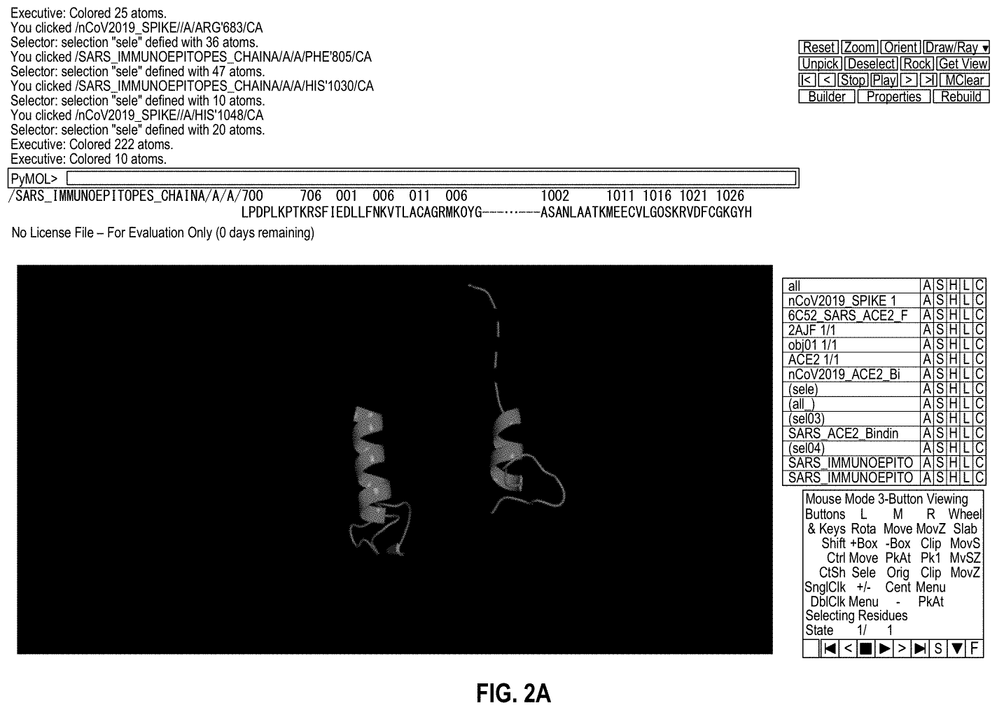

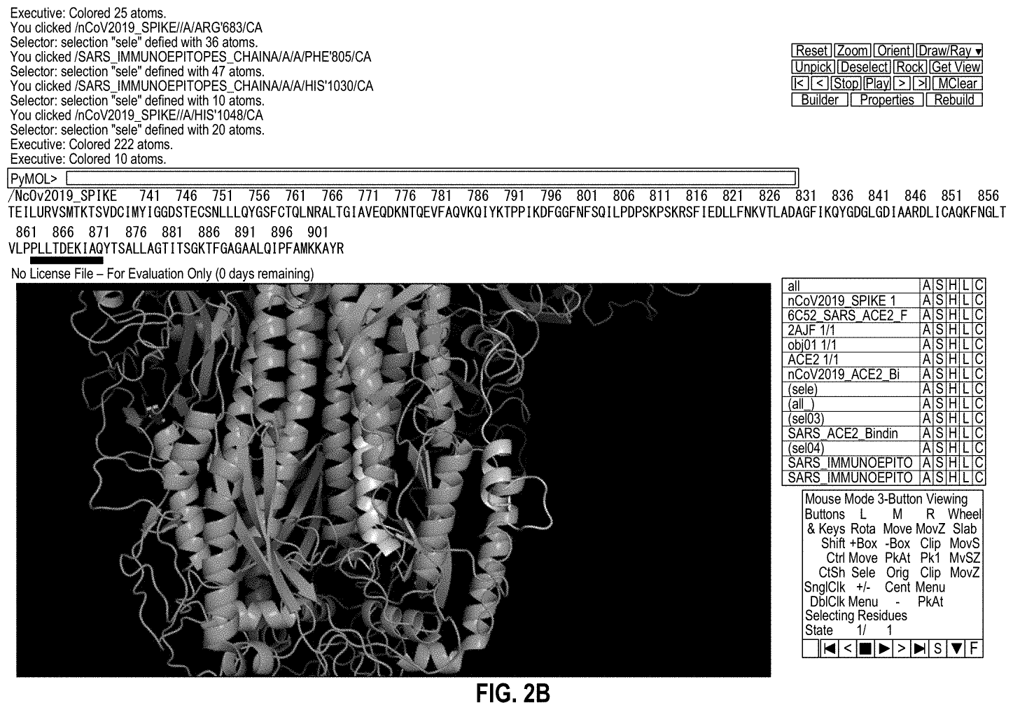

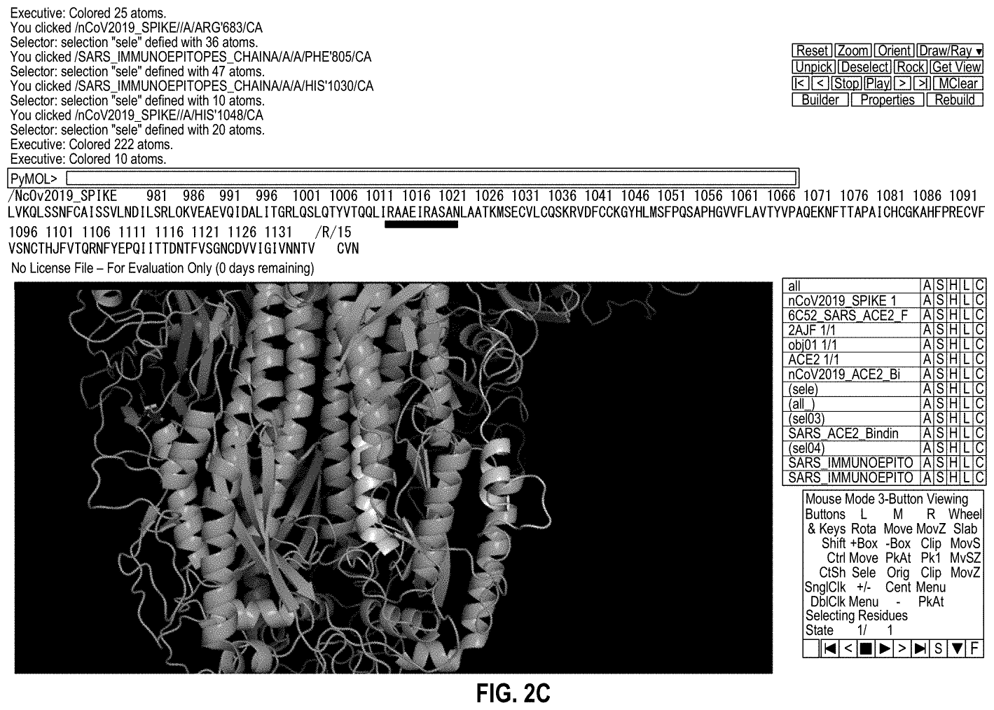

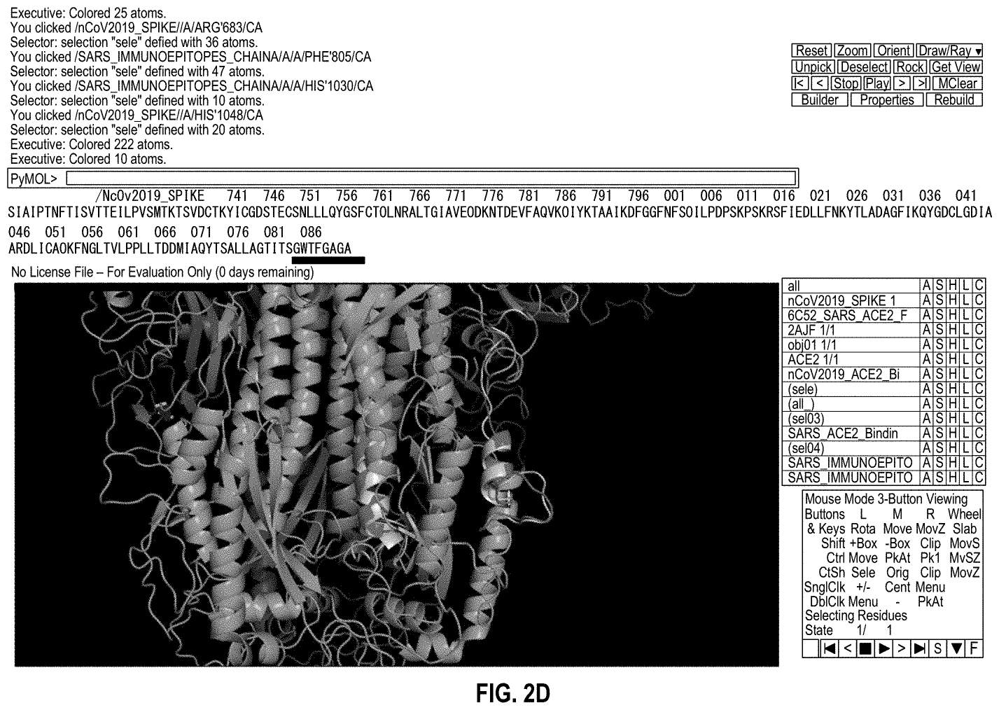

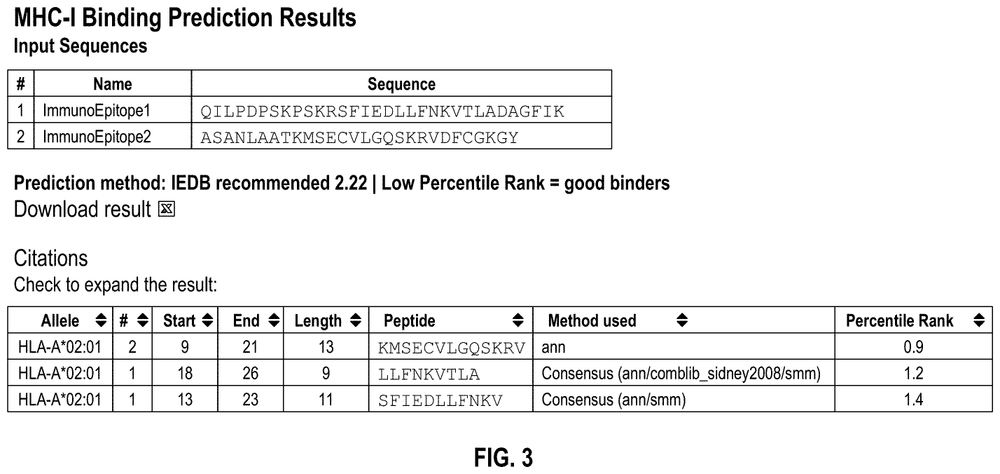

The 3D structure of the SARS-CoV-1 immuno-epitopes are shown in FIG. 2A, and the 3D structure of the CoV-2 immuno-epitopes and their locations on CoV-2 S protein are shown in FIGS. 2B-2D. IEDB determined that sequences KMSECVLGQSKRV (SEQ ID NO:71) and LLFNKVTLA (SEQ ID NO:7) of SARS-CoV-2 S protein, representing MHC-II and MHC-I binding domains for HLA-A*02:01, respectively, would be immunogenic with percentile ranks of 0.9 and 1.2, respectively. Lower percentile rank represents better binding. FIG. 3 shows the MHC-I binding prediction results of these immuno-epitopes. Accordingly, the immuno-epitopes having the following sequences are used in further studies and included in some scaffolds:

(SEQ ID NO: 71)
KMSECVLGQSKRV,
and
(SEQ ID NO: 7)
LLFNKVTLA.

Additional epitopes can be identified from various databases. For example, in the TepiTool results, YLQPRTFLL (SEQ ID NO:9), FIAGLIAIV (SEQ ID NO:22), and FVFLVLLPL (SEQ ID NO:21) are the top scoring HLA-A*0201 epitopes. These top-scoring epitopes are very hydrophobic. Some of the top-scoring epitopes, or alternately if available, epitopes that demonstrate immunogenicity in vivo or in vitro can be included in the scaffolds disclosed herein.

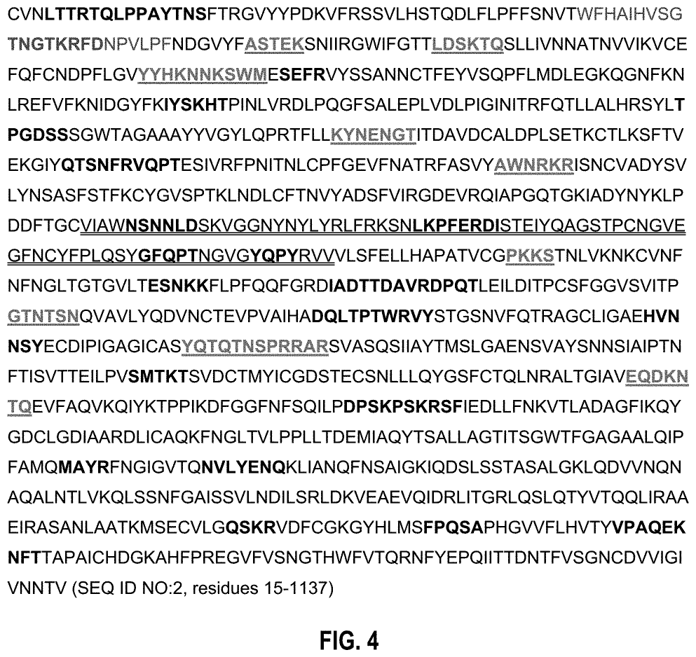

FIG. 4 shows that the antibody epitopes such as B cell epitopes in CoV-2 S protein identified by others (12) are aligned to the CoV-2 S protein amino acid sequence.

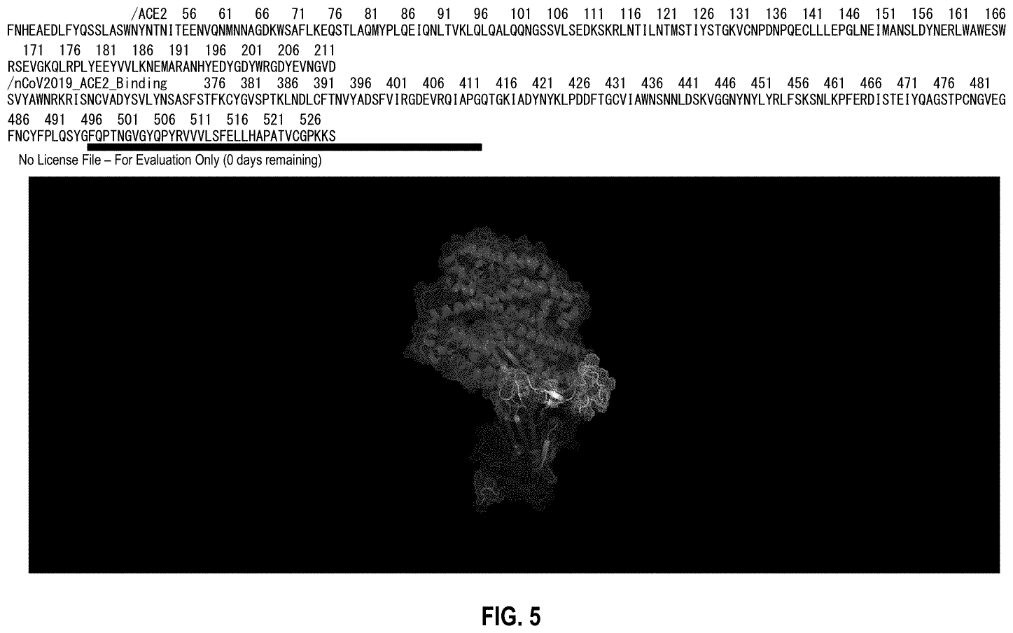

As shown in FIG. 5, the truncated CoV-2 S protein having the amino acid sequence of SEQ ID NO:4 (below) is aligned to ACE2 to show the locations of antibody epitopes (magenta), and ACE2 binding residues (green).

(SEQ ID NO: 4; some immune-epitopes
highlighted in bold).
CPFGEVFNATRFASVYAWNRKRISNCVADYSVLYNSASFS
TFKCYGVSPTKLNDLCFTNVYADSFVIRGDEVRQIAPGQT
GKIADYNYKLPDDFTGCVIAWNSNNLDSKVGGNYNYLYRL
FRKSNLKPFERDISTEIYQAGSTPCNGVEGFNCYFPLQSY
GFQPTNGVGYQPYRVVVLSFELLHAPATVCGPKKST.

Sequence search using the Bepipred tool indicates that most of the receptor-binding motif (residues 440-501) is predicted as being a B cell linear epitope.

PDB can be used to identify B-cell epitopes as well. For example, PDB lists eight epitopes which were previously explored by experiments. Two linear epitopes on the SARS-CoV-2 S protein were demonstrated to elicit neutralizing antibodies in COVID-19 patients (12). Some examples of the B-cell epitopes include: PSKPSKRSFIEDLLFNKV (S21P2) (SEQ ID NO:30), TESNKKFLPFQQFGRDIA (S14P5) (SEQ ID NO:25), PATVCGPKKSTNLVKNKC (SEQ ID NO:24), GIAVEQDKNTQEVFAQVK (SEQ ID NO:26), NTQEVFAQVKQIYKTPPI (SEQ ID NO:27), PIKDFGGFNFSQILPDPS (SEQ ID NO:29), PINLVRDLPQGFSALEPL (SEQ ID NO:23), and VKQIYKTPPIKDFGGFNF (SEQ ID NO:28).

As disclosed in detail below, the scaffolds disclosed herein can be modified to include one or more immuno-epitopes including T cell epitopes and/or B cell epitopes.

These results demonstrate that binding pockets can be predicted in a way that is consistent with Cryo-EM and other high-resolution structural data. This technique can be used to rapidly address future mutations of any known or new viruses, even when genomic data of the entire virus suggests as little as 80% similarity. The technology disclosed herein also incorporates a bioinformatics-driven approach for mapping TCR and BCR/antibody epitopes, allowing for a “compression algorithm” of protein size. In contrast to recombinant technique and other approaches, the technology disclosed herein utilizes a small peptide, such as a peptide of fewer than 70 amino acids out of an about 1200 amino-acid spike protein, to generate a multi-functional scaffold for ACE2 binding and TCR/antibody recognition.

### II. Scaffold/Peptide Modifications

Disclosed herein are CoV-2 scaffolds or ACE2 scaffolds comprising one or more fragments of amino acid sequence from the binding interface of each of CoV-2 S protein and ACE2 while substantially maintaining the structure and/or conformation of the native protein in its free or bound state. The scaffolds disclosed herein substantially maintain or improve the binding affinity to the corresponding binding partner. For example, the CoV-2 scaffolds disclosed herein substantially maintain or improve the binding affinity to wildtype ACE2; and the ACE2 scaffolds disclosed herein substantially maintain or improve the binding affinity to wildtype CoV-2 S protein. The CoV-2 scaffold or the ACE2 scaffold comprises from about 10 to about 100 amino acid residues, from 15 to about 30 amino acid residues, from about 55 to about 95 amino acid residues, from about 60 to about 90 amino acid residues, from about 65 to about 85 amino acid residues, from about 70 to about 80 amino acid residues. In some embodiments, the CoV-2 scaffold or the ACE2 scaffold comprises about 50 amino acid residues, about 55 amino acid residues, about 60 amino acid residues, about 65 amino acid residues, about 70 amino acid residues, about 75 amino acid residues, about 80 amino acid residues, about 85 amino acid residues, about 90 amino acid residues, about 95 amino acid residues, or about 100 amino acid residues. In some embodiments, the CoV-2 scaffold or ACE2 scaffold comprises two or more sequences that enhance binding, displacement or immunogenicity of the scaffold(s). In some embodiments, the viral-mimetic or pathogen-mimetic scaffolds need not be related to SARS-CoV-2 and its binding to ACE2, and can be derived from any pathogen binding to its concomitant human or host protein binding partner(s), including eukaryote and prokaryote species.

In certain embodiments, disclosed herein is a CoV-2 scaffold or an ACE2 scaffold, each comprising two critical binding motifs, wherein the critical binding motifs are involved with direct binding to the binding partner. In some embodiments, the scaffold further comprises one or more backbone regions comprising amino acid residues not involved with direct binding to the binding partner. In some embodiments, the backbone region is located between the two critical binding motifs. In some embodiments, the backbone region is located at the N-terminus of the first critical binding motif. In some embodiments, the backbone region is located at the C-terminus of the second critical binding motif.

In certain embodiments, the CoV-2 scaffold disclosed herein comprises a first critical binding motif having at least 50%, at least 55%, at least 60%, at least 65%, at least 70%, at least 75%, at least 80%, at least 85%, at least 90%, at least 95%, at least 98%, or 100% identity to an amino acid sequence of NSNNLDSKVGGNYNYLYRL (SEQ ID NO:65), and a second critical binding motif having at least 50%, at least 55%, at least 60%, at least 65%, at least 70%, at least 75%, at least 80%, at least 85%, at least 90%, at least 95%, at least 98%, or 100% identity to an amino acid sequence of

(SEQ ID NO: 66)
YQAGSTPCNGVEGFNCYFPLQSYGFQPTNGVGYQP.

In certain embodiments, the ACE2 scaffold disclosed herein comprises a first critical binding motif having at least 50%, at least 55%, at least 60%, at least 65%, at least 70%, at least 75%, at least 80%, at least 85%, at least 90%, at least 95%, at least 98%, or 100% identity to an amino acid sequence of STIEEQAKTFLDKFNHEAEDLFYQ (SEQ ID NO:149), and a second critical binding motif having at least 50%, at least 55%, at least 60%, at least 65%, at least 70%, at least 75%, at least 80%, at least 85%, at least 90%, at least 95%, at least 98%, or 100% identity to an amino acid sequence of NAGDKWSAFLKEQSTLAQMYP (SEQ ID NO:150).

The scaffolds disclosed herein may have different sizes depending on the number of the amino acid residues from the backbone included between the two critical binding motifs, at the N-terminus of the first critical binding motif and/or at the C-terminus of the second critical binding motif. See, for example, Scaffold #1 (SEQ ID NO:72) (top) and Scaffold #10 (SEQ ID NO:81) (bottom), amino acid sequences aligned below.

SEQ ID NO: 72
VIAWNSNNLDSKVGGNYNYLYRLFRKSNLKPFERDISTEIYQAGSTPCNG
VEGFNCYFPLQSYGFQPTNGVGYQPYRVV
SEQ ID NO: 81
----NSNNLDSKVGGNYNYLYRLFRKSNLKPFERDISTEIYQAGSTPCNG
VEGFNCYFPLQSYGFQPTNGVGYQPY---

In certain embodiments, one or more amino acid residues in the scaffold are deleted or substituted. For example, one or more repulsory amino acid residues having a positive ΔG are deleted or substituted, one or more neutral amino acid residues having a ΔG of about 0 are deleted or substituted, and/or one or more amino acid residues outside of the critical binding motifs, e.g., in the backbone region, are deleted or substituted. Although not desirable, one or more critical amino acid residues having a negative ΔG can be deleted or substituted.

In certain embodiments, the scaffold is modified by replacing the amino acid residues in the backbone region between the critical binding motifs with a linker such as a GS linker having various lengths. In some embodiments, the scaffold comprises a linker having about 1 to about 20 amino acid residues, for example, the linker has 1, 2, 3, 4, 5, 6, 7, 8, 9, 10, 11, 12, 13, 14, 15, 16, 17, 18, 19, or 20 amino acid residues. One can optimize the size of the linker to achieve a desired structure and/or conformation of the scaffold. See, for example, Scaffold #1 (SEQ ID NO:72), Scaffold #3 (SEQ ID NO:74), Scaffold #11 (SEQ ID NO:82), Scaffold #12 (SEQ ID NO:83), Scaffold #13 (SEQ ID NO:84), and Scaffold #14 (SEQ ID NO:85), from top to bottom in the order of appearance, amino acid sequences aligned below. The GS linkers are shown in bold.

```
VIAWNSNNLDSKVGGNYNYLYRLFRKSNLKPFERDISTEIY
QAGSTPCNGVEGENCYFPLQSYGFQPTNGVGYQPYRVV
VIAWNSNNLDSKVGGNYNYLYRLGSGSG
QAGSTPCNGVEGFNCYFPLQSYGFQPTNGVGYQPYRVV
        NSNNLDSKVGGNYNYLYRLGSGSGS
QAGSTPCNGVEGFNCYFPLQSYGFQPTNGVGYQPY
        NSNNLDSKVGGNYNYLYRLGSGSG
QAGSTPCNGVEGFNCYFPLQSYGFQPTNGVGYQPY
        NSNNLDSKVGGNYNYLYRLGSGS
QAGSTPCNGVEGFNCYFPLQSYGFQPTNGVGYQPY
        NSNNLDSKVGGNYNYLYRLGSG
QAGSTPCNGVEGFNCYFPLQSYGFQPTNGVGYQPY
```

In certain embodiments, the scaffold comprises one or more immuno-epitopes such as one or more T cell epitopes, one or more B cell epitopes, or both. The immuno-epitopes can be included within a non-interfacing loop structure which replaces the entire or partial sequence of the backbone region between the two critical binding motifs of the scaffold. For example, one or more amino acid residues in the backbone region can be replaced by one or more immuno-epitopes. In another example, one or more amino acid residues in the critical binding motif, preferably, the repulsory or neutral amino acid residues, can be replaced by one or more immuno-epitopes. Depending on the desirable size and structure of the scaffold, one can choose which amino acid residues to be replaced by one or more immuno-epitopes.

In certain embodiments, the immuno-epitopes are 9 or 13 amino acid residues long corresponding to MHC-I and MHC-II binding. For example, the T cell epitopes include but are not limited to KMSECVLGQSKRV (SEQ ID NO:8), and LLFNKVTLA (SEQ ID NO:7). Other known epitopes may be included in the scaffold as well. For example, dominant TCR epitopes including KLWAQCVQL (SEQ ID NO:10) (ORF1ab, 3886-3894, 17.7 nM, mostly for A*02), YLQPRTFLL (SEQ ID NO:9) (S, 269-277, 5.4 nM, mostly for A*02), and LLYDANYFL (SEQ ID NO:11) (ORF3a, 139-147, mostly for A*02) (3). Other known TCR epitopes include PRWYFYYLGTGP (SEQ ID NO:12) (nucleocapsid), SPRWYFYYL (SEQ ID NO:13) (nucleocapsid, mostly for B*07:02, A*11:01, A*03:01), WSFNPETN (SEQ ID NO:14) (membrane protein), QPPGTGKSH (SEQ ID NO:15) (ORF1ab polyprotein), and VYTACSHAAVDALCEKA (SEQ ID NO:16) (ORF1ab polyprotein) (1, 4-6). Some epitopes are strong but may be HLA restricted such as KTFPPTEPK (SEQ ID NO:17) (N protein; 20.8 nM), CTDDNALAYY (SEQ ID NO:18) (ORF1ab; 5.3 nM), TTDPSFLGRY (SEQ ID NO:19) (ORF1ab; 7.2 nM), and FTSDYYQLY (SEQ ID NO:20) (ORF3a; 3.2 nM).

In certain embodiments, the B cell epitopes include but are not limited to FDEDDS (SEQ ID NO:63), IQKEIDRL (SEQ ID NO:62), KYFKNHTSP (SEQ ID NO:61), MAYR (SEQ ID NO:56), NVLYENQ (SEQ ID NO:57), QSKR (SEQ ID NO:58), YQPY (SEQ ID NO:45), SEFR (SEQ ID NO:36), TPGDSS (SEQ ID NO:38), TTKR (SEQ ID NO:64), YYHKNNKSWM (SEQ ID NO:35), ASTEK (SEQ ID NO:33), AWNRKR (SEQ ID NO:41), DPSKPSKRSF (SEQ ID NO:55), DQLTPTWRVY (SEQ ID NO:50), EQDKNTQ (SEQ ID NO:54), ESNKK (SEQ ID NO:47), FPQSA (SEQ ID NO:59), GFQPT (SEQ ID NO:44), GTNTSN (SEQ ID NO:49), HVNNSY (SEQ ID NO:51), IADTTDAVRDPQT (SEQ ID NO:48), IYSKHT (SEQ ID NO:37), KYNENGT (SEQ ID NO:39), LDSKTQ (SEQ ID NO:34), LKPFERDI (SEQ ID NO:43), LTTRTQLPPAYTNS (SEQ ID NO:31), NSNNLD (SEQ ID NO:42), PKKS (SEQ ID NO:46), QTSNFRVQPT (SEQ ID NO:40), SMTKT (SEQ ID NO:53), TNGTKRFD (SEQ ID NO:32), VPAQEKNFT (SEQ ID NO:50), and YQTQTNSPRRAR (SEQ ID NO:52). The locations of the B cell epitopes in CoV-2 S protein are shown in FIG. 4.

See, for example, Scaffold #10 (SEQ ID NO:81), Scaffold #15 (SEQ ID NO:86), Scaffold #16 (SEQ ID NO:87), Scaffold #17 (SEQ ID NO:88), Scaffold #18 (SEQ ID NO:89), Scaffold #19 (SEQ ID NO:90), and Scaffold #20 (SEQ ID NO:91), from top to bottom in the order of appearance, amino acid sequences aligned below. Scaffold #1 (SEQ ID NO:72), Scaffold #3 (SEQ ID NO:74), Scaffold #11 (SEQ ID NO:82), Scaffold #12 (SEQ ID NO:83), Scaffold #13 (SEQ ID NO:84), Scaffold #14 (SEQ ID NO:85), from top to bottom in the order of appearance, amino acid sequences aligned below. Amino acid residues in the critical binding motifs are underlined, and the immuno-epitopes are shown in bold. Depending on the desired size and/or structure of the scaffold, the immuno-epitopes can replace the entire or partial sequence of the backbone region between the critical binding motifs, and/or can replace partial sequence of the critical binding motif, in particular the repulsory and/or neutral amino acid residues in the critical binding motif.

NSNNLDSKVGGNYNYLYRLFRKSNLKPFERDISTEI
YQAGSTPCNGVEGFNCYFPLQSYGFQPTNGVGYQPY
NSNNLDSKVGGNYNYLYRLKMSECVLGQSKRV
QALLFNKVTLAGFNCYFPLQSYGFQPTNGVGYQPY
NSNNLDSKVGGNYNYLYRLFRKMSECVLGQSKRV
QALLFNKVTLAGFNCYFPLQSYGFQPTNGVGYQPY
NSNNLDSKVGGNYNYLYRLFRKSKMSECVLGQSKRV
QALLFNKVTLAGFNCYFPLQSYGFQPTNGVGYQPY
NSNNLDSKVGGNYNYLYRLFRKSNKMSECVLGQSKRV
QALLFNKVTLAGFNCYFPLQSYGFQPTNGVGYQPY
NSNNLDSKVGGNYNYLYRLFRKSNL
KMSECVLGQSKRVQALLFNKVTLA
GFNCYFPLQSYGFQPTNGVGYQPY
NSNNLDSKVGGNYNYLYRLFRKSNLK
KMSECVLGQSKRVQALLFNKVTLA
GFNCYFPLQSYGFQPTNGVGYQPY

In certain embodiments, the scaffold comprises one or more Cys substitutions such that a Cys-Cys bridge can be formed at a desired location via a disulfide bond. For example, L455C and P491C substitutions are made to introduce a Cys-Cys bridge to maintain or stabilize the 13 sheet structure of the scaffold. In some embodiments, the Cys residues at other locations can be substituted by Gly or other residues to avoid interference of Cys-Cys bridge at the desired location. In other embodiments, other click chemistry or diselenide chemistry techniques can be used to bridge two amino acids or monomeric regions of the scaffold(s) to recreate a desired structure.

In certain embodiments, the scaffold further comprises a head and/or a tail comprising one or more charged amino acids such as poly(Arg), poly(Lys), poly(His), poly(Glu) or poly(Asp) attached to the N-terminus, C-terminus, or both. These cationic or anionic sequences are added to make an electrostatic nanoparticle of the scaffolds disclosed herein.

In certain embodiments, the scaffold comprises one or more amino acid substitutions to increase ACE2 binding affinity, antibody affinity, or both. For example, substitutions that increase ACE2 binding affinity include but are not limited to: N439R, L452K, T470N, E484P, Q498Y, N501T. For example, substitutions that alter antibody affinity include but are not limited to: A372T, S373F, T393S, 1402V, S438T, N439R, L441I, S443A, G446T, K452K, L455Y, F456L, S459G, T470N, E471V, Y473F, Q474S, S477G, E484P, F490W, Q493N, S494D, Q498Y, P499T, and N501T. Substitutions that increase ACE2 binding affinity while decreasing or potentially displacing antibody binding and B cell binding to those sequences can contribute to immune evasion and immune escape. In certain embodiments, the scaffold comprises one or more amino acid substitutions include N501Y, N501T, E484K, S477N, T478K, L452R, and N439K, sequences and other snippets of sequences that need not necessarily be from the S-protein, versus an active sequence that has selection pressure for enhanced pathogenicity or transmissibility, or contributes to antigenic drift/escape. These peptides can be rapidly designed and distributed in advance of worldwide spread to cover specific zones as new strains emerge, and this principle can also be applied to other pathogens (including bacteria, fungi, protozoans, etc.) and viruses. Some exemplary pathogenic variants that enhance ACE2 binding, which may or may not correspondingly increase infectivity, pathogenicity, and antibody escape. For example, N426-F443 and Y460-Y491 can be maintained.

In certain embodiments, the scaffold comprises a His tag or a C-tag having an amino acid sequence of EPEA.

The scaffolds disclosed herein can be linear peptides. Alternatively, the scaffolds disclosed herein can be cyclic peptides, for example, the linear peptides can be head-to-tail cyclized via an amide bond. Some examples of the head-to-tail cyclic scaffolds include Scaffold #43 (SEQ ID NO:114) and Scaffold #44 (SEQ ID NO:115) having the following amino acid sequences (GS linker in bold):

(SEQ ID NO: 114)
SNNLDSKVGGNYNYLYRLGSGSG
QAGSTPCNGVEGFNCYFPLQSYGFQPTNGVGYQP;
and
(SEQ ID NO: 115)
SNNLDSKVGGNYNYLYRCGSGSG
QAGSTPGNGVEGFNGYFCLQSYGFQPTNGVGYQP.

These cyclic scaffolds are predicted to be non-aggregating and non-toxic and have a binding affinity equivalent to or better than the linear scaffolds. In certain embodiments, alternative linkers can be used to further optimize the cyclic scaffolds. Some examples of the head-to-tail cyclic scaffolds have the following amino acid sequences (linker in bold):

(Scaffold #45; SEQ ID NO: 116)
SNNLDSKVGGNYNYLYRLFDGTEIYQAGSTPCNGVEGFNCY
FPLQSYGFQPTNGVGYQP;
and
(Scaffold #53; SEQ ID NO: 124)
SNNLDSKVGGNYNYLYRLFPKPEIYQAGSTPCNGVEGFNCY
FPLQSYGFQPTNGVGYQP.

In certain embodiments, additional amino acid residues can be added to the scaffold to achieve a desired size or structure. Likewise, these scaffolds can be linear peptides or head-to-tail cyclic peptides. Some examples of the scaffolds have the following amino acid sequences (added residues shown in bold):

(Scaffold #46; SEQ ID NO: 117)
SNNLDSKVGGNYNYLYRLFNANDEIYQAGSTPCNGVEGFN
CYFPLQSYGFQPTNGVGYQP;
(Scaffold #47; SEQ ID NO: 118)
SNNLDSKVGGNYNYLYRLFNAHDKIYQAGSTPCNGVEGFN
CYFPLQSYGFQPTNGVGYQP;
(Scaffold #48; SEQ ID NO: 119)
SNNLDSKVGGNYNYLYRLFNANDKIYQAGSTPCNGVEGFN
CYFPLQSYGFQPTNGVGYQP;
and
(Scaffold #49; SEQ ID NO: 120)
SNNLDSKVGGNYNYLYRLFDAHDKIYQAGSTPCNGVEGFN
CYFPLQSYGFQPTNGVGYQP.

In certain embodiments, the scaffold is modified to include a linker comprising a Pro residue to obtain a more rigid structure. Some examples of such rigid scaffolds have the following amino acid sequences (Pro-containing linker shown in bold):

(Scaffold #50; SEQ ID NO: 121)
SNNLDSKVGGNYNYLYRLFPKPEQAGSTPCNGVEGFNCYFP
LQSYGFQPTNGVGYQP;
(Scaffold #51; SEQ ID NO: 122)
SNNLDSKVGGNYNYLYRLFPGTEIYQAGSTPCNGVEGFNCY
FPLQSYGFQPTNGVGYQP;
(Scaffold #52; SEQ ID NO: 123)
SNNLDSKVGGNYNYLYRLFPATEIYQAGSTPCNGVEGFNCY
FPLQSYGFQPTNGVGYQP;
(Scaffold #54; SEQ ID NO: 125)
SNNLDSKVGGNYNYLYRLFPGTDIYQAGSTPCNGVEGFNCY
FPLQSYGFQPTNGVGYQP;
and
(Scaffold #55; SEQ ID NO: 126)
SNNLDSKVGGNYNYLYRLFPAHDKIYQAGSTPCNGVEGFNC
YFPLQSYGFQPTNGVGYQP.

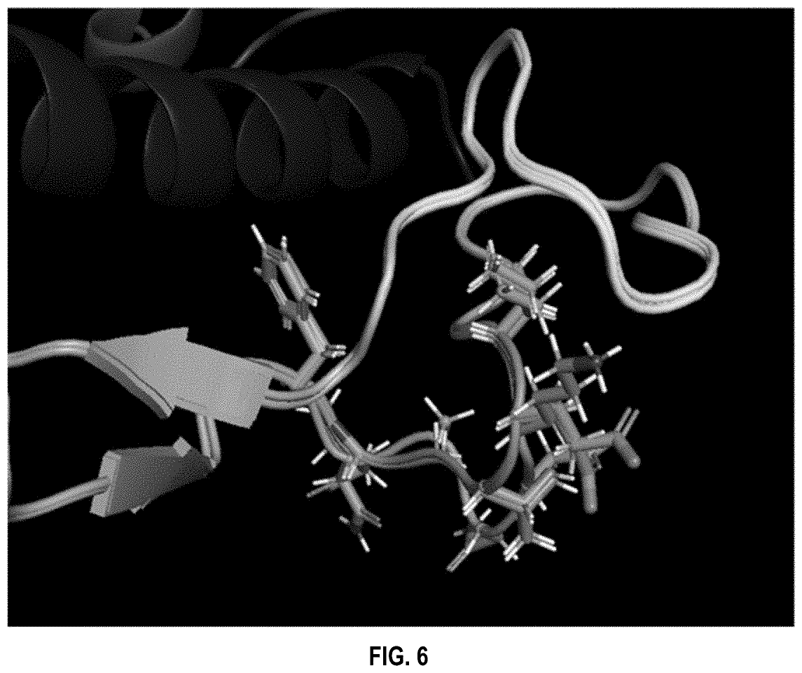

FIG. 6 depicts the three-dimensional molecular modeling of three representative linkers in the bound conformation.

In certain embodiments, the scaffold comprises a PEG chain such as PEG2000 (45-unit) to allow binding to both units of dimeric ACE2. In some embodiments, the PEG chain has a length of between 30 and 60 unit, between 35 and 55 units, or between 40 and 50 units. In some embodiments, the PEG chain has a length of about 30, about 31, about 32, about 33, about 34, about 35, about 36, about 37, about 38, about 39, about 40, about 41, about 42, about 43, about 44, about 45, about 46, about 47, about 48, about 49, about 50, about 51, about 52, about 53, about 54, about 55, about 56, about 57, about 58, about 59, or about 60 units.

In certain embodiments, the scaffold comprises one or more amino acid substitutions with hydrophilic amino acids or polymeric sequences to reduce aggregation. In certain embodiments, the scaffold comprises modifications to increase the number of hydrophilic amino acids. In certain embodiments, the scaffold is configured with hydrophobic amino acids facing inward. In some embodiments, PEG, poly(sarcosine), or hydrophilic polymer sequences can be added to increase scaffold solubility. Some examples of such scaffolds have the following amino acid sequences (K substitution shown in bold):

(Scaffold #41; SEQ ID NO: 112)
VKAWNSNNLDSKVGGNYNYLYRLGSGSGQAGSTPCNGV
EGFNCYFPLQSYGFQPTNGVGYQPYRVV;
and
(Scaffold #42; SEQ ID NO: 113)
VIKWNSNNLDSKVGGNYNYLYRLGSGSGQAGSTPCNGV
EGFNCYFPLQSYGFQPTNGVGYQPYRVV.

The scaffolds disclosed herein can be joined to one or more additional scaffolds or other peptides using an appropriate linker to generate multimeric structures. In certain embodiments, dimers may be formed by linking two scaffolds or peptides together with a linker. In certain embodiments, trimers may be formed by linking three scaffolds or peptides with two linkers. Larger multimeric structures, e.g., assemblies including four, five, six, seven, or eight scaffolds or peptides linked together may be generated. Examples of linkers include, but are not limited to, PEG or poly(sarcosine).

In certain embodiments, the scaffold comprises a native F residue at the N-terminus, residues IYQ at the C-terminus, or both. In some embodiments, the scaffold comprises a closing of the head-to-tail cyclic peptide at the residues YQP.

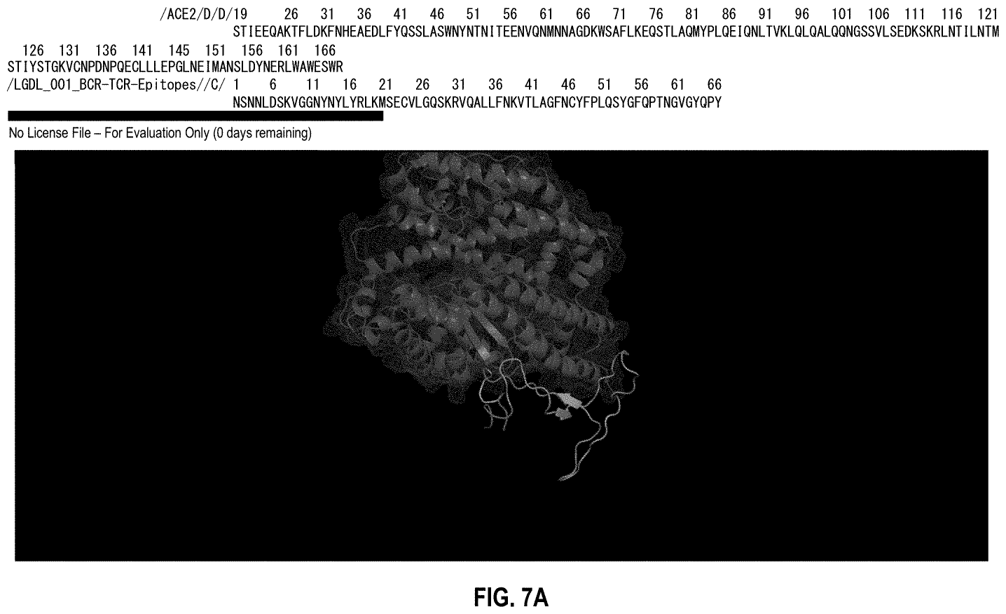

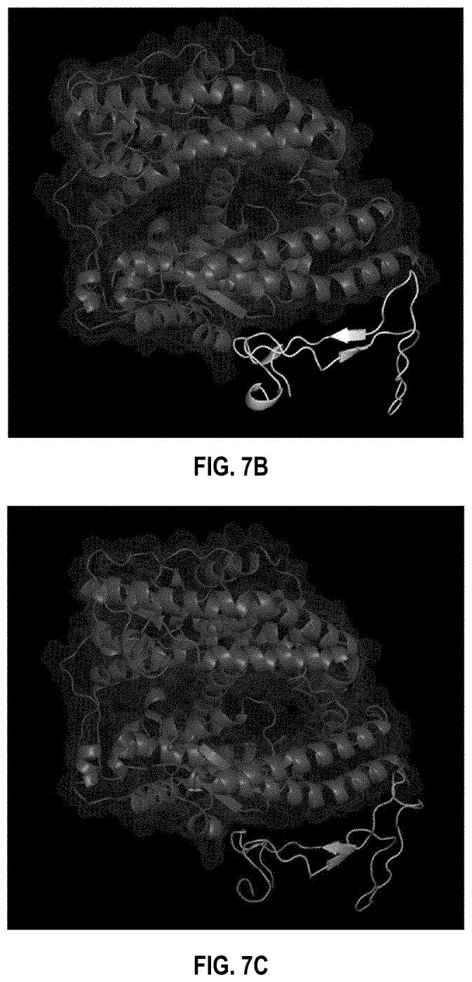

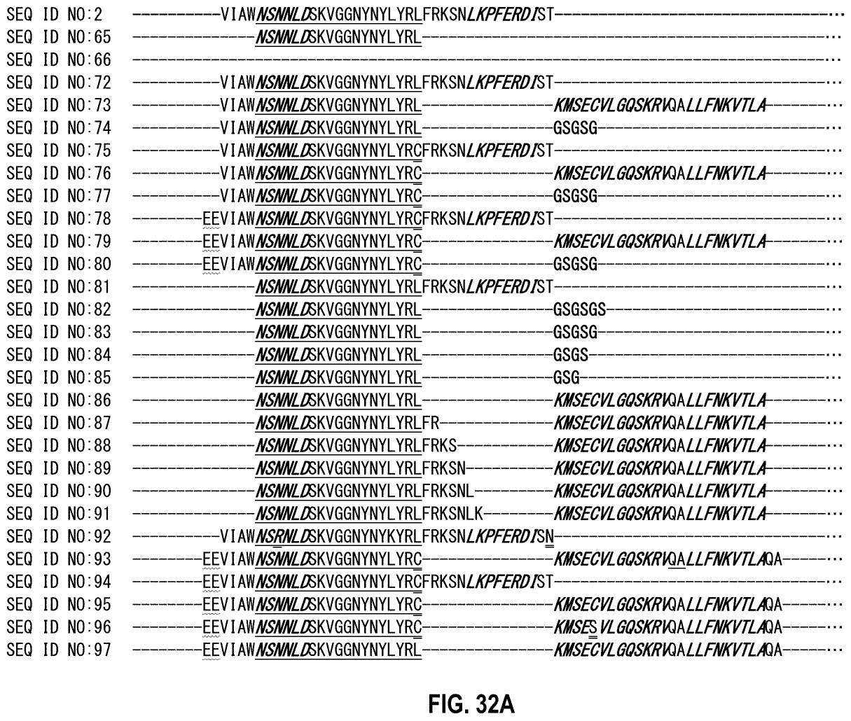

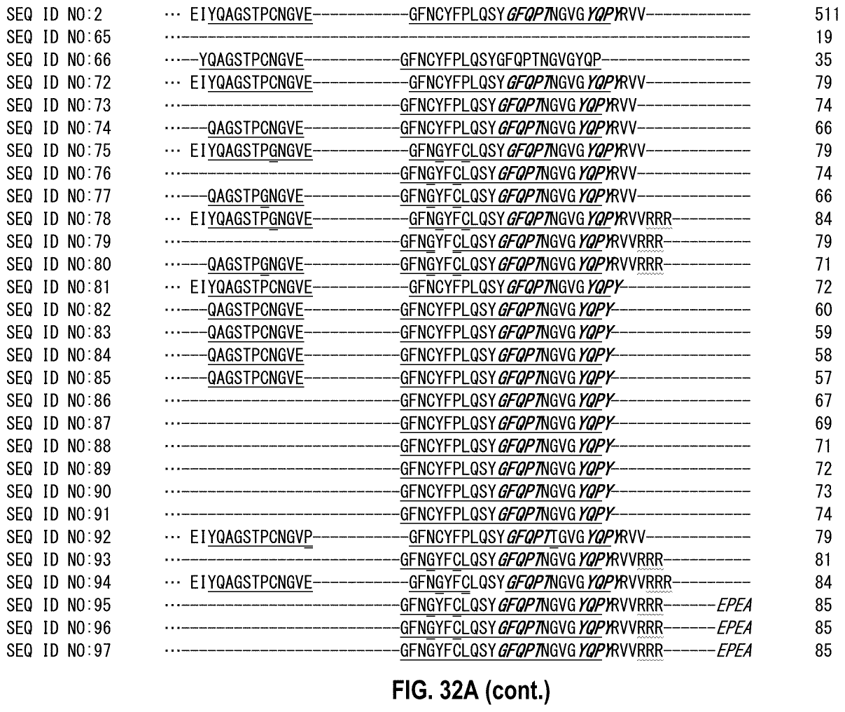

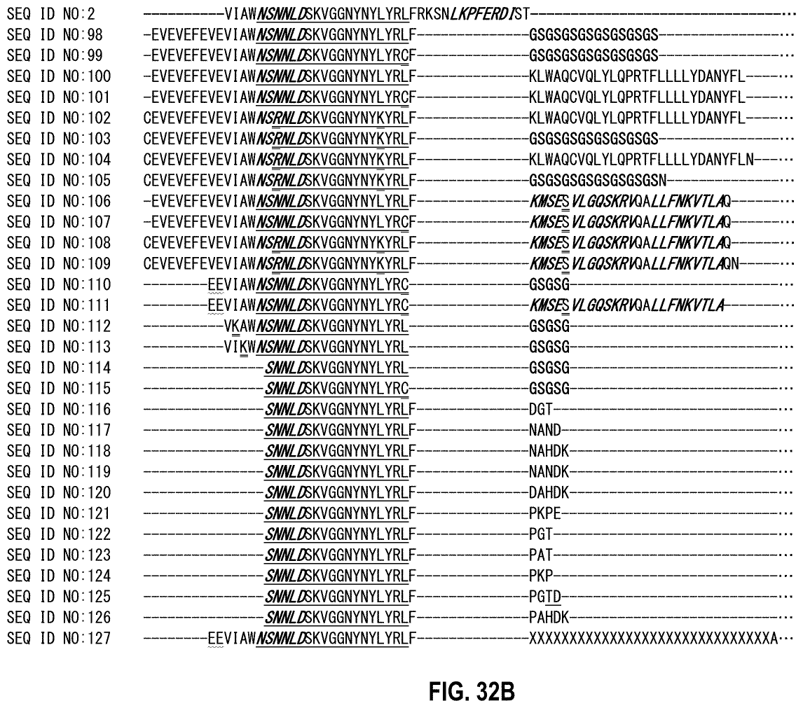

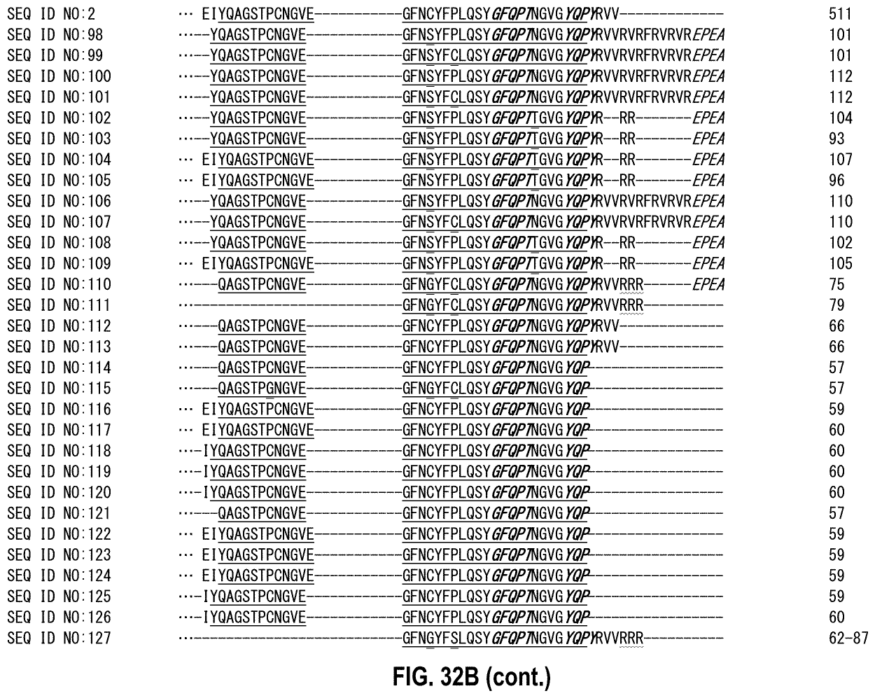

Representative examples of scaffolds derived from SARS-CoV-2 S protein are set forth in Table 1 below. Critical binding motifs are underlined, immune-epitopes are bolded and italicized, and linkers are bolded. Alignments of these representative scaffold sequences are set forth in FIGS. 32A-32B. The obtained scaffolds can be fitted to ACE2 to investigate the binding affinity, as shown in FIGS. 7A-7C.

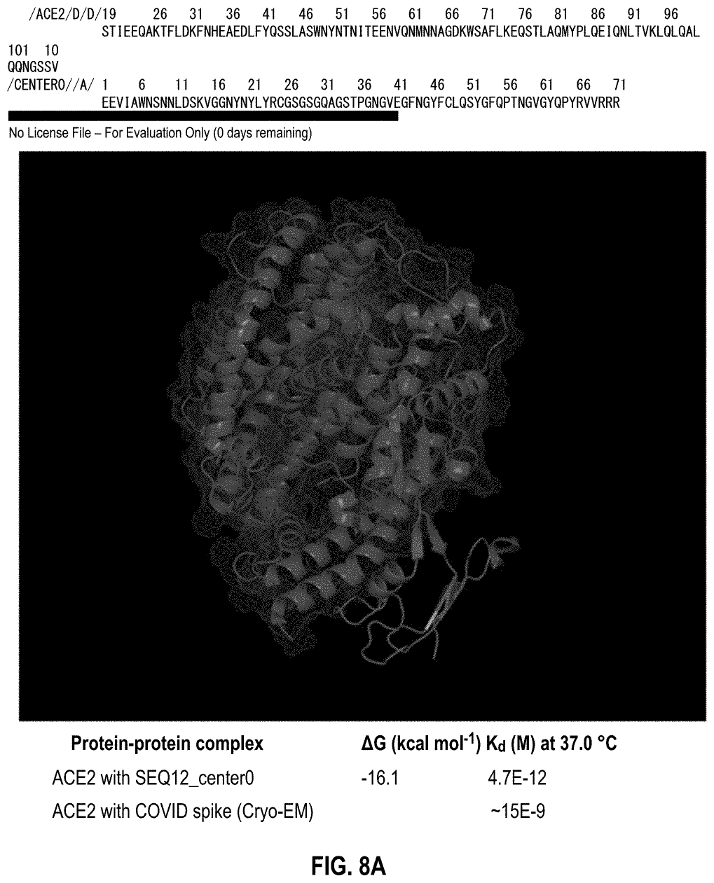

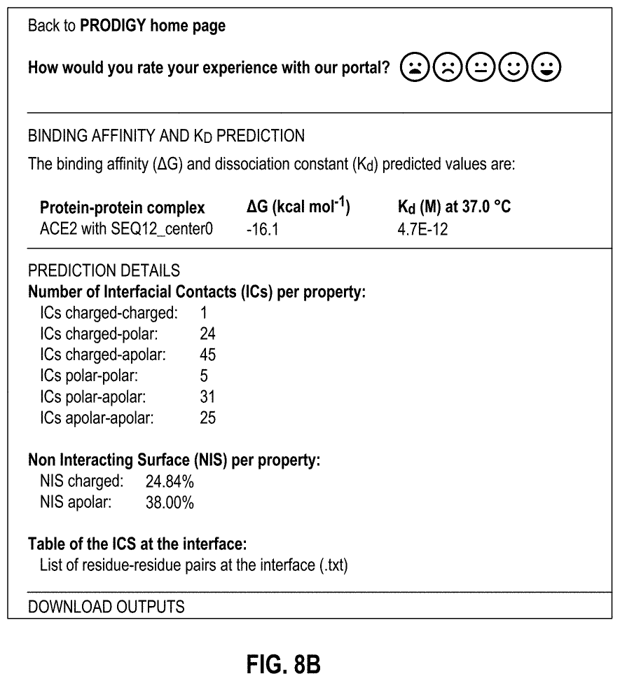

As shown in FIGS. 8A-8B, the CoV-2 scaffolds, with or without modifications, can be fitted to ACE2 to determine their ACE2 binding affinity and Ko prediction based on computer modeling.

The selected scaffolds are subject to further structural analysis and modification to achieve higher binding affinity, better efficacy, and/or improved stability. The scaffolds disclosed herein, with or without modifications, can be obtained by any existing technology, for example, by peptide synthesis or by recombinant technology.

### III. In Vitro Assay of Scaffolds

Biolayer interferometry assay is performed, as demonstrated in the working examples, to screen for scaffolds having high binding affinity to ACE2 and significant inhibition of CoV-2 infection in vitro. Biolayer interferometry (“BLI”) is a method for measuring the wavelength shift of incident white light following loading of a ligand upon a sensor tip surface, and/or binding of a soluble analyte to that ligand on the sensor tip surface. The wavelength shift corresponds to the amount of an analyte present and can be used to determine dissociation constants and competition between multiple analytes and the immobilized ligand period the wavelength shift corresponds to the amount of an analyte presents and can be used to determine dissociation constants and competition between multiple analytes and an immobilized ligand.

Specifically, interactions of the scaffolds with ACE2 and SARS-CoV-2 neutralizing antibodies are characterized by biolayer interferometry, as well as by pseudotyped lentiviral infection of ACE2-HEK293 cells. As demonstrated in the working examples, a statistically significant inhibition of infection was observed with doses as low as 30 nM of Scaffold #8 (SEQ ID NO:79), with 95% or greater inhibition of infection in the 6.66 μM range.

ACE2, commonly known as the viral entry receptor for SARS-CoV-2, exists in both membrane-bound and soluble forms. While ACE2 prevents infection in vitro when presented in soluble form, it may contribute to the immune cloaking and immunoevasive properties of the virus in vivo, essentially shielding the spike protein in its open conformation from recognition by the adaptive immune system. As demonstrated in the working example, a statistically significant inhibition of SARS-CoV-2 pseudotyped lentiviral infections was observed with ACE2 concentrations as low as 4 nM. Yet the working examples further demonstrate that soluble ACE2 prevented neutralizing antibodies from binding to the spike protein's receptor binding domain (RBD).

In patients with heart failure, soluble ACE2 exists in plasma concentrations of 16.6-41.1 ng/mL (1st and 4th quartile ranges), which corresponds to approximately 193-478 μM, while some studies report concentrations of 7.9 ng/mL in acute heart failure patients and 4.8 ng/mL in healthy volunteers, which corresponds to approximately 92 and approximately 56 μM, respectively (4, 6).

Other studies report that male and female patients with type 1 diabetes (approximately 27.0 ng/mL) with comorbidities of diabetic nephropathy (approximately 25.6 ng/mL) and/or coronary heart disease (approximately 35.5 ng/mL) had higher circulating ACE2 concentrations than male controls (approximately 27.0 ng/mL), with higher arterial stiffness and microvascular or macrovascular disease being positively associated with soluble ACE2 concentrations (14). In such ranges, ACE2 may enhance infection in vivo due to occluding the receptor binding domains of the S1 spike protein in open conformation, given that an individual virus spike only takes on this “open” conformation after exposure to furin (during biosynthesis) and TMPRSS2 (during membrane association) (7; 17). Additionally, the higher concentrations of ACE2 in patients with cardiovascular, diabetic, renal, and vascular disease may further be associated with increased pathogenicity of SARS-CoV-2. Because ACE2 exhibits extremely potent binding affinity for the SARS-CoV-2 receptor binding domain (RBD), which may interfere with neutralizing antibody binding to the virus, the virus may avoid detection by the immune system as a function of soluble ACE2. SARS-CoV-2 viral titers in the blood of clinical specimens are lower relative to bronchoalvaeolar lavage, fibrobronchoscope brush biopsy, sputum, nasal swab, pharyngeal swab, and feces (an average of 2^{4.6} reduction versus a cycle threshold of 30 corresponding to <2.6×10⁴ copies/mL), corresponding to approximately 1000 viral copies per mL in the blood (20). Assuming about 100 spikes per virus, this corresponds to approximately 100,000 possible ACE2-binding sites per mL of blood if all spikes are in open conformation. However, given that the open conformation only occurs after TMPRSS2 cleavage, the starting position of each spike must be assumed as being closed, and likely only a fraction of these about 100,000 sites are exposed for ACE2 or neutralizing antibody binding at any given point in time. Therefore, an approximately 193-478 μM soluble ACE2 concentration corresponds to 1.6×10¹⁴ to 2.9×10¹⁴ molecules/mL, which, when coupled to the approximately 720 μM to 1.2 nM Kd of ACE2 to the spike protein in open conformation, suggests that SARS-CoV-2 would primarily exist with its “open” spikes occluded by ACE2 in blood. ACE2 is predicted to bind to certain SARS-CoV-2 RBD mutants with as little as 110 to 130 μM Kd and, importantly—when in fully “open” conformation—the SARS-CoV-2 spike protein exhibits comparable binding affinity to neutralizing antibodies that compete for this same binding site (25; 26). This is particularly troubling when considering the ability of ACE2 to hinder neutralizing antibody binding to this site, and that neutralizing antibodies are a product of B cell maturation, whereby B cells must mature antibodies and BCRs to reach single-digit nanomolar or picomolar binding affinities comparable in strength to ACE2-spike binding.

Indeed, SARS-CoV-2 has a binding affinity for ACE2 that is comparable to that of even potently neutralizing antibodies, and according to results provided herein. As demonstrated herein, ACE2 severely abrogates antibody binding to the SARS-CoV-2 spike RBD as well as serving as potent inhibitor of infection of SARS-CoV-2 pseudotyped lentivirus in ACE2-expressing cells in vitro. Together, these data indicate that ACE2 serves both a protective function against infection and inhibitory function on immune recognition of the virus, acting as a competitive inhibitor of neutralizing antibody recognition against the spike protein, with binding affinities ranging from about 676 μM to about 33.97 nM (1).

As demonstrated in the working examples, the receptor binding domain (RBD) of SARS-CoV-2 spike bound to ACE2 with an affinity of about 3 nM, and ACE2 was able to prevent association of a neutralizing antibody with the RBD that would otherwise have about a binding affinity of about 6 nM. In sum, ACE2 binding to “open” conformation spike proteins is a viable mechanism at physiological ACE2 concentrations for inhibiting neutralizing antibody formation and binding against the spike protein RBD, and the virus has multiple mechanisms for avoiding detection by neutralizing antibodies as a result.

The most recent spike protein mutation, D614G, seems to further increase the density of “open” spike proteins on the surface versus the original sequence, as well as the density of spikes in general, which notably makes this mutant likely to be more sensitive to neutralizing antibodies versus the aspartic acid (D) containing variant, while also increasing infectivity (9; 23). In fact, the D614G variant seems to display >½ log¹⁰ (˜3×) increased infectivity in ACE2-expressing cells with SARS-CoV-2 pseudotyped lentiviral infection assays (11).

As SARS-CoV-2 and COVID19 continue to ravage the world, it will be important to monitor the emergence and susceptibility of various mutants to “immune cloaking” by avoidance of neutralizing antibody recognition or recognition of the spike protein in “open” conformation.

### IV. Applications of Scaffolds/Peptides and Compositions Comprising the Same

Also disclosed herein are compositions comprising one or more scaffolds, a conjugate comprising one or more scaffolds, or a fusion protein comprising one or more scaffolds. In some embodiments, the composition further comprises one or more pharmaceutically acceptable carriers, excipients, or diluents. In some embodiments, the composition can be formulated into an injectable, inhalable, oral, nasal, topical, transdermal, uterine, lubricant, oil, candy, gummy bear, and/or vaginal and rectal dosage form. In some embodiments, the composition is administered to a subject by a parenteral, oral, pulmonary, buccal, nasal, transdermal, rectal, vaginal, catheter, urethral, or ocular route.

As disclosed in this document, the scaffolds can be modified by adding a polar head or a polar tail comprising 2-150 amino acid residues, e.g., comprising poly(Arg), poly(K), poly(His), poly(Glu), or poly(Asp) to the N-terminus or the C-terminus, as well as non-natural amino acids and other polymeric species including glycopeptides, polysaccharides, linear and branched polymers, and the like. Examples of non-natural amino acids and other polymeric species that may be suitable for use with the scaffolds of the present disclosure include polymeric molecules described in U.S. Provisional Patent Application No. 62/889,496, which is incorporated herein by reference. A recombinant membrane-fusion domain may also be added to the scaffolds via a linker. Thus, the scaffolds can be assembled into electrostatic nanoparticles. Additionally, the scaffolds can be immobilized on chips for surface plasmon resonance (SPR). The scaffolds can serve as ligands for targeted delivery for various therapeutics such as siRNAs, CRISPR based technology and small molecules as part of both synthetic and naturally/recombinantly derived delivery systems, gene and protein-based payloads, and the like. The scaffolds can be either synthetic or recombinant, and can include linkers and synthetic or recombinant modifications to the N-terminus or the C-terminus to further enhance membrane fusion or delivery substrate fusion. Optionally, the targeted delivery can be nanoparticle-based. Various tags known in the art can be attached to the scaffolds as well, e.g., His-tag and C-tag.

Also disclosed in this document, the scaffolds may comprise a loop which allows attachment of a conjugatable domain using the existing peptide conjugate technology. In some embodiments, the scaffold disclosed herein may be conjugated via maleimide, which is commonly used in bioconjugation, and which reacts with thiols, a reactive group in the side chain of Cys residue. Maleimide may be used to attach the scaffolds disclosed herein to any SH-containing surface as illustrated below:

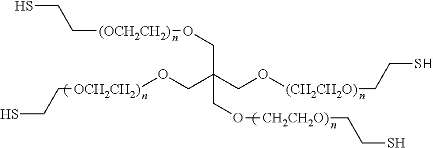

The scaffolds disclosed herein may comprise one or more immuno-epitopes. Further, one or more scaffolds disclosed herein may be conjugated together via linkers or other conjugatable domains to obtain multi-epitope, multi-valent scaffolds. Additionally, the scaffolds disclosed herein can be attached to other immune-response eliciting domains or fragments. In some embodiments, one or more of the scaffolds disclosed herein can be attached to an Fc fragment to form a fusion protein.

Both the ACE-2 scaffolds and the CoV-2 scaffolds disclosed herein can be used in compositions as a “coating” to block or inhibit virus entry. In some embodiments, the ACE-2 scaffolds can bind to the RBD of the CoV-2 virus to coat the virus thereby to block virus entry into human body. In some embodiments, the CoV-2 scaffolds can bind to the ACE2 binding domain to coat the ACE2 receptor thereby to block virus entry into human body.

The scaffolds disclosed herein are small peptides having a size of less than 100 amino acids, e.g., about 70 amino acid residues or less and comprising: 1) immuno-epitopic regions for T cell receptor MHC-I and MHC-II loading, 2) immuno-epitopic regions for B cell receptor or antibody binding, and 3) ACE2 receptor binding regions. Not only can these synthetic or recombinant scaffolds serve as competitive inhibitors for ACE2 binding by the SARS-CoV-2 virus, they are also designed to trigger immune learning and be able to be presented on a variety of immunologically active scaffolds and adjuvants. Additionally, these scaffolds can readily be conjugated to a variety of immunoadjuvants as well as known and novel substrates for multivalent display. These scaffolds may also be used for a variety of infectious disease-causing agents, ranging from bacteria, fungi, protozoans, amoebas, parasites, viruses, sexually-transmitted diseases, and the like.

Additionally, the disclosed technology allows for targeted delivery of a variety of therapeutic agents, including silencing RNAs, CRISPR and other gene editing based technologies, and small molecule agents to virally-infected cells. For example, the scaffolds disclosed herein can serve as a ligand for nanoparticle-based siRNA delivery and small molecule conjugate approaches in therapeutic design and development. Further, the scaffolds comprising immuno-epitopes can also present key residues for immuno-epitopic recognition by antibodies and T cell receptors through MHC-I and MHC-II loading as determined by predicted antibody binding regions for the most distal loop structures of the entire SARS-CoV-2 protein based on crystal structure data of SARS-CoV-1 with a neutralizing antibody, in addition to IEDB immuno-epitope prediction approaches.

Due to their binding to neutralizing antibodies against the RBD, the scaffolds disclosed herein are also expected to enhance immune response to SARS-CoV-2 rather than blunting it. In contrast, approaches such as ACE2-mimetic and antibody therapies are likely to reduce neutralizing antibody response to the virus, since they coat the virus and prevent binding of the adaptive immune system to the portion that is bound, which is the same segment of the spike protein necessary for B cell receptor (BCR) maturation into neutralizing antibodies targeting the spike protein RBD in its “open” conformation.

Importantly, the scaffolds disclosed herein are not expected to interfere in the activity of ACE2, due to binding to the face of the enzyme that does not metabolize angiotensin II. Critically for vaccine design and immune response promotion, these peptides are also designed to have modular epitopes for MHC-I and MHC-II recognition, which can be customized to the haplotypes of various patient populations, in addition to the inclusion of antibody-binding epitopes within the peptide sequences. Promisingly, recovered COVID-19 patients form dominant CD8+ T cell responses against a conserved set of epitopes, with 94% of 24 screened patients across 6 HLA types exhibiting T cell responses to 1 or more dominant epitopes, and 53% of patients exhibiting responses to all 3 dominant epitopes (5; 27). Furthermore, previous studies demonstrate that patients with various HLA genotypes form MHC-I mediated responses to varying SARS-CoV-2 epitopes, and this can be predicted with bioinformatics approaches (10). While bioinformatic predictions of MHC loading corresponding to various HLA genotypes do not predictively reveal which peptide sequences will or will not be loaded, they do create a comprehensive overview of the possible state-spaces for empirical validation. The scaffolds disclosed herein are designed to display modular motifs for priming clonal expansion of selective TCR repertoire, which can be facilitated by sequencing of recovered patient TCR repertoires and insertion into these scaffolds and assessing HLA genotypes of target populations (28). This affords a facilitated method for rapid vaccine and antidote design, coupling bioinformatics with structural and patient-derived omics data to create an iterative design approach to treating infectious agents.

The in vitro studies provide proof of principle for antibody recognition and effective viral blockade. The synthetic nature of the scaffolds affords utility in tethering these peptides to a variety of substrates via click chemistry, which include but are not limited to C60 buckminsterfullerene, single and multi-walled carbon nanotubes, dendrimers, traditional vaccine substrates such as KLH, OVA and BSA, and the like—though the bare alkyne-terminated peptides are examined in the present example. The synthetic nature, in silico screening, and precise conformation of these peptides allows for rapid synthesis without traditional limitations of recombinant, live-attenuated, gene delivery system, viral vector, or inactivated viral vaccine approaches. Due to the click chemistry nature of these peptides, they may also serve as drug and gene delivery carriers by modifications with electrostatic sequences, or by click chemistry or membrane fusion onto lipidic particles. Compositions provided herein comprising the scaffolds disclosed herein and future permutations of these peptides may be used to facilitate the design, development and scale-up of precise therapeutic agents and vaccines against a variety of infectious agents as part of broader biodefense initiatives. The peptides need not be synthetic, and may optionally comprise recombinant variants or a fusion between a recombinant protein and a moiety selected from the group consisting of synthetic peptides, polymers, peptoids, glycoproteins, polysaccharides, lipopeptides, and liposugars.

The scaffolds disclosed herein are designed to overcome many limitations associated with antibody therapies, ACE2-Fc therapies, and other antiviral therapeutics. Though neutralizing antibodies may be used as “stopgap” therapeutics to prevent the progression of disease, the transient nature of administered antibodies leaves the organism susceptible to reinfection. Furthermore, as demonstrated in the present example, ACE2 is a potent inhibitor of neutralizing antibody binding to the SARS-CoV-2 spike protein receptor binding domain. Therapeutics that mimic ACE2 and shield this key epitope are likely to bias antibody formation towards off-target sites, which could contribute to antibody-dependent enhancement (ADE), vaccine-associated enhanced respiratory distress (VAERD), and a host of other immunological issues upon repeat viral challenge. These key issues are also important to consider in vaccine development, as there is precedent for enhanced respiratory disease in vaccinated animals with SARS-CoV-1 (29). With SARS-CoV-1, a marked lack of peripheral memory B cell responses was observed in patients 6 years following infection (30). Thus, any approach that promotes a specific and neutralizing immune response, whether freestanding or in conjunction with another vaccine approach or infection, should be considered as an alternative to immunosuppressant and potentially off-target antibody forming approaches.

In particular, any approaches that have potential to limit endogenous antibody formation should be carefully reconsidered, due to the viral immuno-evasive techniques spanning a gamut of mechanisms, including but not limited to the spike protein switching between “open” and “closed” conformations, heavy glycosylation limiting accessible regions, and also the presentation of T cell evasion due to MHC downregulation on infected cells and potential MHC-II binding of the SARS-COV-2 spike protein limiting CD4+ T cell responses, which all may be factors in contributing to T cell exhaustion and ineffective and/or transient antibody and memory B cell responses in infected patients. An ideal therapeutic strategy should enhance neutralizing antibody formation, not blunt it, while also preventing the virus from entering cells and replicating(31; 32; 33; 34). Indeed, severe and critically ill patients exhibit extreme B cell activation and, presumably, antibody responses. Yet, poor clinical outcomes are seen, suggesting that immune evasion and/or off-target antibody formation is dominant (35; 36).

The extent to which various factors individually play in contributing to this phenomenon remains poorly understood. Surely, COVID19 presents itself as a multifactorial disease with a cascade of deleterious effects. Also, the potential for reinfection across cohorts of varying disease severity remains to be fully elucidated, though numerous clinical and anecdotal reports indicate that immunity to coronaviruses is markedly short-lived, with seasonal variations in susceptibility to reinfection with alpha- and beta-coronaviruses being frequently observed, and some antibody responses lasting for no longer than 3 months (37).

With SARS-CoV-2 in particular, patients developing moderate antibody responses are seen to have undetectable antibodies in as little as 50 days (38). Additionally, one study on 149 recovered individuals reported that 33% of study participants did not generate detectable neutralizing antibodies 39 days following symptom onset, and that the majority of the cohort did not have high neutralizing antibody activity (39).

Importantly, results provided herein indicate that the scaffolds disclosed herein can be used as both therapeutic agents and vaccines due to the presence of key epitopes for antibody formation, and the performance of Scaffold #8 (SEQ ID NO:79) which exhibits one MHC-I epitope and one MHC-II epitope in these experiments. The MHC-I and MHC-II domains can be flexibly substituted to match HLA types in various populations or pooled across panels of peptides exhibiting multiple domains. Because the disclosed scaffolds mimic the virus, rather than binding to it, and also due to its ability to displace ACE2 from cloaking the virus spike protein, compositions provided herein may prove to be an effective immune-enhancing strategy in infected patients, with additional potential to serve as a prophylactic vaccine.

Therefore, the scaffolds disclosed herein and the compositions comprising the same can be used to prevent viral association with ACE2 and infection, while also contribute to a decrease in soluble ACE2 shielding of the virus. The thermodynamically favorable interaction of an antibody with the virus (about 6 nM Ko with the neutralizing antibody studied herein) versus scaffolds provided herein (about 1 μM Ko) suggests that the scaffolds can dissociate ACE2, promote antibody formation against the virus during infection, and preferentially train the immune system to eliminate the virus.

### Example 1. Simulation and Docking of SARS-CoV-2 Spike (S) Protein in the Absence of Structural Data

This working example demonstrates building a structural simulation of the novel virus SARS-CoV-2 using SWISS-MODEL based on a SARS-CoV-2 spike protein sequence (UniProt ID P0DTC2) and its homology to SARS-CoV-1 (PDB ID 6CS2) in the absence of crystallographic or cryo-EM data determining the atomic-resolution structure of SARS-CoV-2 and in the absence of any data on the binding cleft of the CoV-2 virus to the ACE2 receptor.

To elucidate the binding motif of the CoV-2 receptor binding domain (RBD), in the absence of structural data, the results of prior crystallography experiments on SARS-CoV-1 with ACE2 were relied upon. SWISS-MODEL was utilized to generate a SARS-CoV-2 spike protein structure prior to the availability of Cryo-EM or X-ray crystallography data in February of 2020 (20-3).

The SARS-CoV-2 spike protein structure was aligned with SARS-CoV-1 spike protein bound to ACE2 (PDB ID 6CS2) using PyMOL (The PyMOL Molecular Graphics System, Version 2.3.5 Schrödinger, LLC.). This structure was then run through PDBePISA to determine the Gibbs free energy (ΔG) and predicted amino acid interactions between the SARS-CoV-2 spike protein and the ACE2 receptor (10). PyMOL was also used to align a truncated sequence of SARS-CoV-1 (locations 322-515) in its native conformation with the ACE2 receptor to SARS-CoV-2 S protein (locations 336-531) and thermodynamic ΔG calculations of the simulated binding pocket of SARS-CoV-2 S protein with ACE2 were performed utilizing PDBePISA. Upon the availability of structural data, this approach was compared and determined to have correctly identified the stretches of amino acids necessary for binding to ACE2, as detailed in Example 4.

As shown in FIG. 1, SARS coronavirus (“SARS-CoV”; “SARS-CoV-1”; or “CoV-1”) protein sequence (PDB ID 6CS2) was compared to SARS-CoV-2 S protein sequence (hereinafter “CoV-2”; SEQ ID NO:2, encoded by nucleotides 21536-25357 of SEQ ID NO:1) and a homology model was generated using SWISS-MODEL, which was then imported into PyMOL as a PDB file.

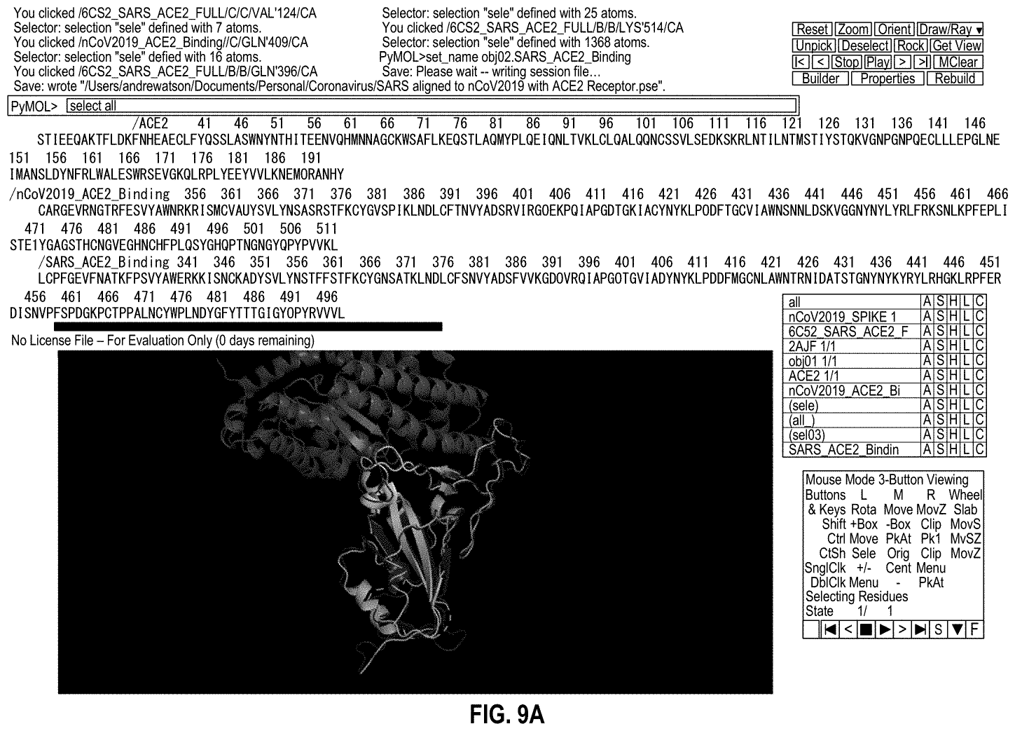

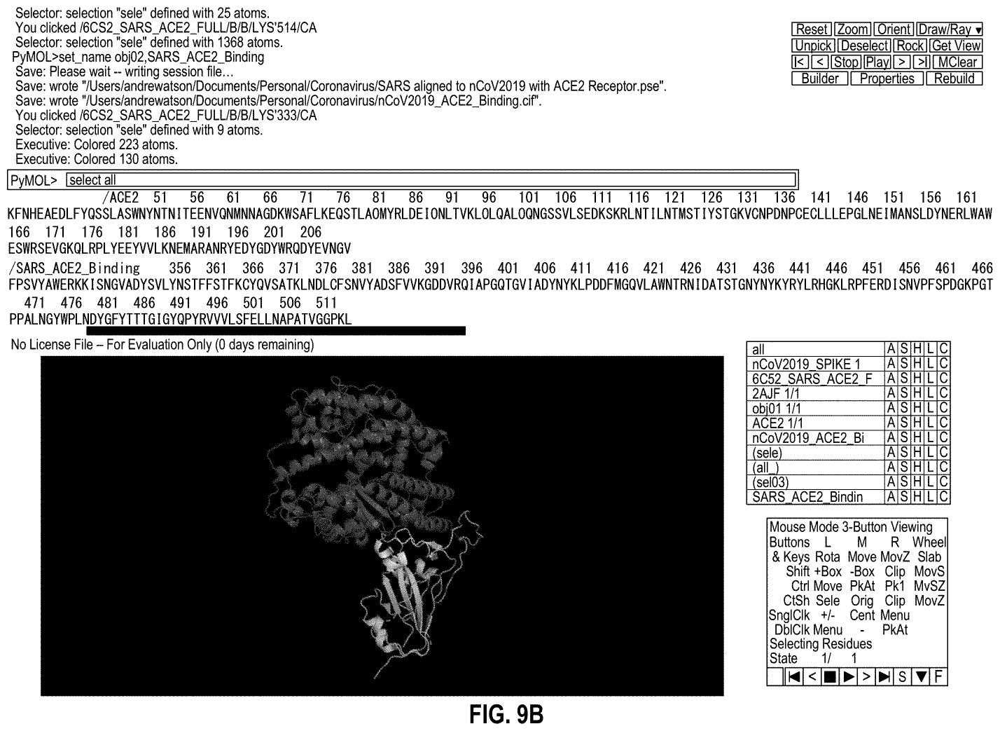

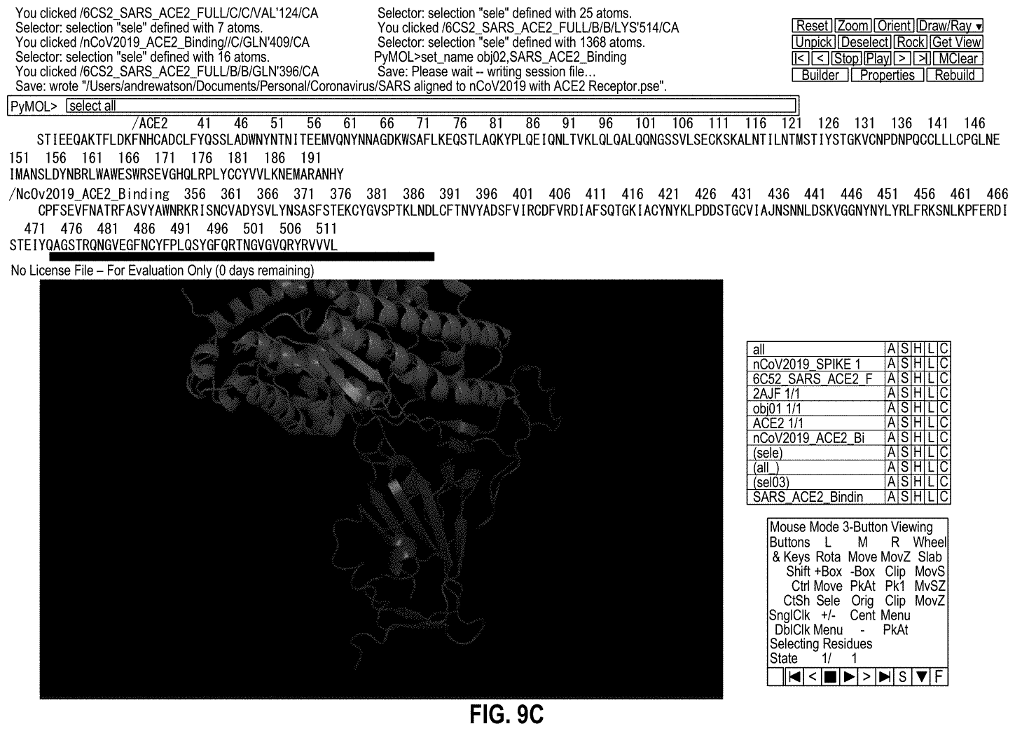

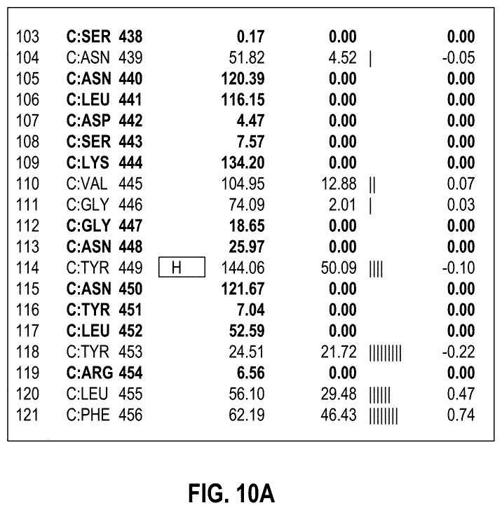

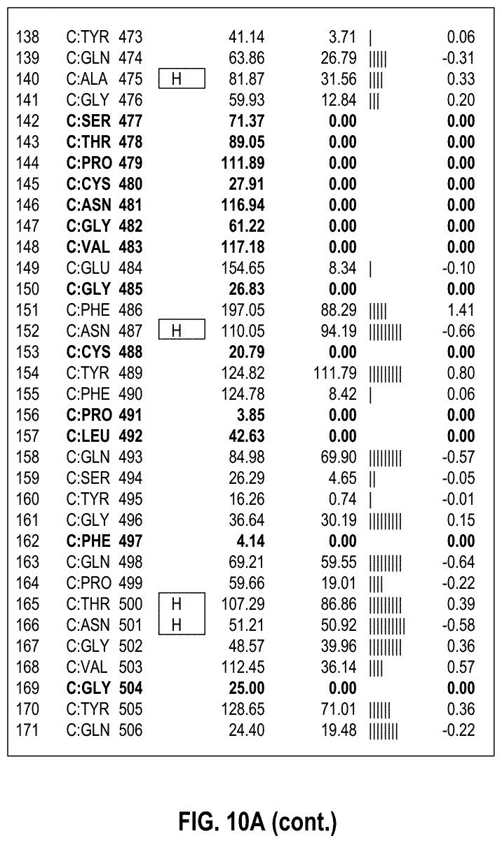

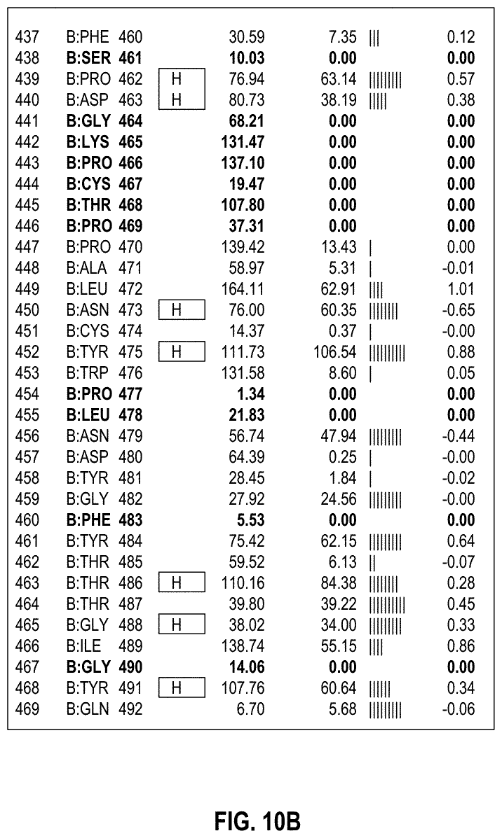

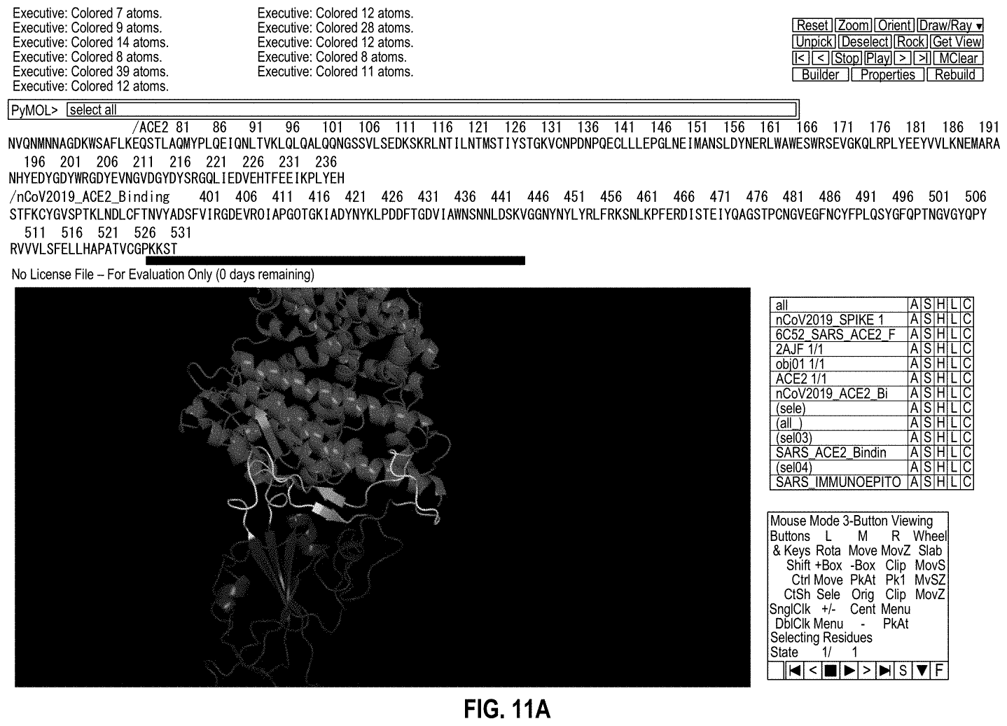

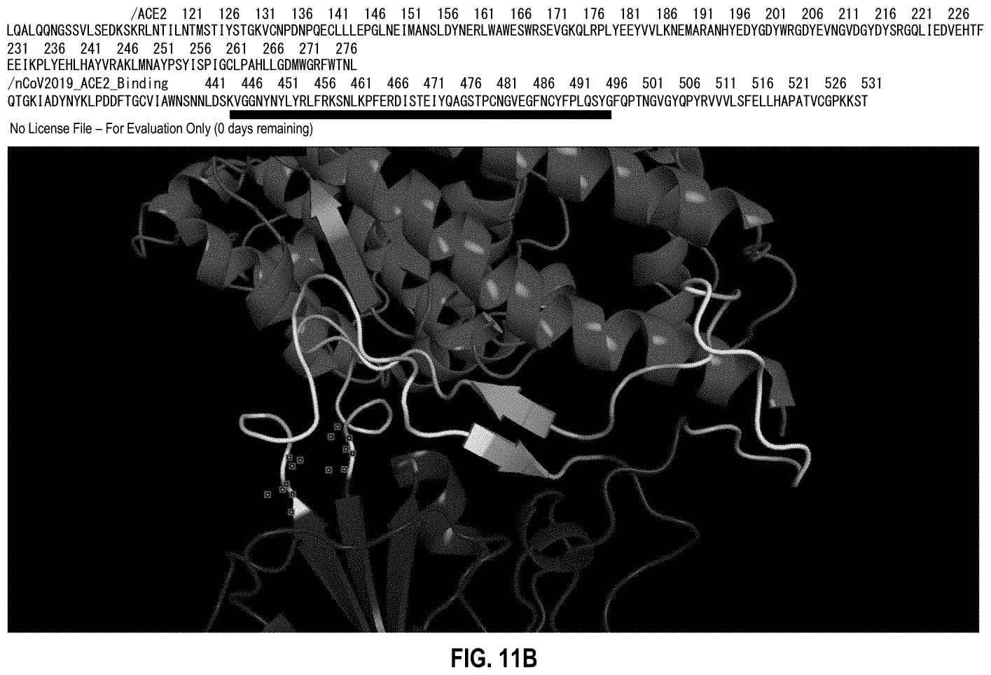

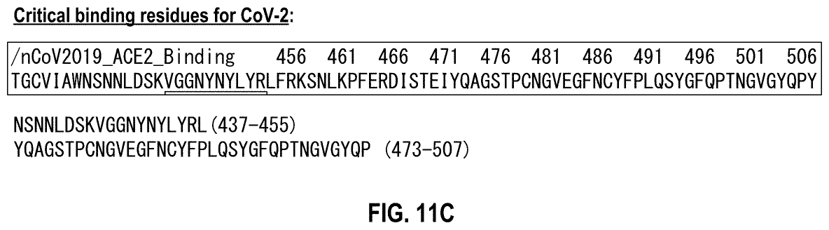

Chain A of CoV-2 was aligned with Chain A of SARS-CoV-1 (PDB ID 6CS2) in the bound state to the ACE2 receptor (see FIGS. 9A-9C). PDB-PISA was run on the binding interface of CoV-2 S protein with the ACE2 receptor to determine the critical binding residues. FIGS. 10A and 10B show the results of ΔG calculation for each residue on the binding interface for CoV-2 and CoV-1, respectively. As shown in FIGS. 11A-11C, the key residues of CoV-2 S protein for binding to ACE2 (negative ΔG) are highlighted in green, while residues having about 0 ΔG are shown in yellow, and repulsory residues have a positive ΔG are shown in orange. L455 and P491, shown in magenta, are in proximity based on the computer model, and therefore, maybe replaced by Cys residues to introduce a disulfide bond between these two locations to stabilize the β sheet structure in the CoV-2 binding interface.

### Example 2. Design and Simulation of Synthetic Peptide Scaffolds Mimicking S Protein Receptor Binding Motif

Following the simulation of structure and binding of the SARS-CoV-2 S protein RBD, a peptide scaffold comprising the truncated receptor binding motif (RBM) was designed. This sequence was designed to recreate the structure of the large protein in this motif, with key modifications performed to facilitate β sheet formation. Various deep learning-based approaches were used to simulate the structure of the peptide scaffolds. For example, a SUMMIT supercomputer-based modeling approach can be used to simulate binding of putative scaffolds. Additionally, a PDBePISA and Prodigy combined approach can be added to the supercomputer's heuristics for assessing binding cleft interactions. The modeling techniques can also include the CD147-SPIKE interactions as components such that the supercomputer's molecular dynamics simulations can predict in the absence of pre-biased alignment. Furthermore, modeling with or without use of the supercomputer can be coupled with the use of RaptorX (or AlphaFold or equivalent) to model a free energy folding state of a random peptide sequence. This allows for combinatorial screening of random peptide sequences using the supercomputer and then running molecular dynamics simulations on a target receptor. These folding techniques are uniquely distinguished from homology modeling, which does not take into account free energy of the peptide and full gamut of possible folded states of a smaller truncated protein fragment, which has different free energy than a larger protein. Approaches provided herein allow for generation of de novo peptide sequences that are then simulated in their folding and binding states.

For illustration, the peptide scaffolds with or without modifications were simulated using RaptorX, which is an efficient and accurate protein structure prediction software package, building upon a powerful deep learning technique (19). Given a sequence, RaptorX is used to run a homology search tool HHblits to find its sequence homologs and build a multiple sequence alignment (MSA), and then derive sequence profile and inter-residue coevolution information (13). Afterwards, RaptorX is used to feed the sequence profile and coevolution information to a very deep convolutional residual neural network (of about 100 convolution layers) to predict inter-atom distance (i.e., Ca-Ca, Cb-Cb and N-O distance) and inter-residue orientation distribution of the protein under prediction. To predict inter-atom distance distribution, RaptorX discretizes the Euclidean distance between two atoms into 47 intervals: 0-2, 2-2.4, 2.4-2.8, 2.8-3.2 . . . 19.6-20, and >20A. To predict inter-residue orientation distribution, RaptorX discretizes the orientation angles defined previously (13) into bins of 10 degrees. Finally, RaptorX derives distance and orientation potential from the predicted distribution and builds 3D models of the protein by minimizing the potential. Experimental validation indicates that such a deep learning technique is able to predict correct folding for many more proteins than ever before and outperforms comparative modeling unless proteins under prediction have very close homologs in Protein Data Bank (PDB).

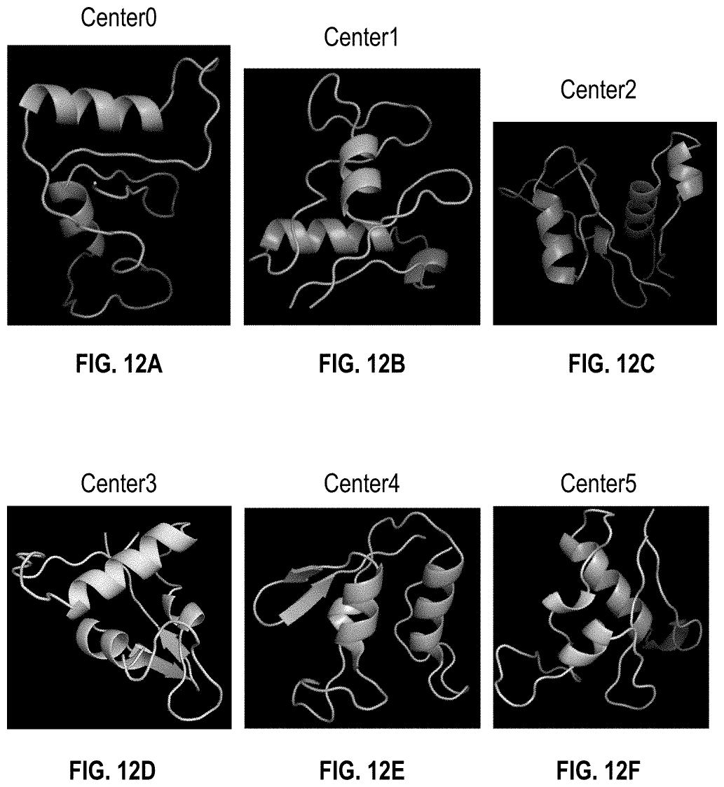

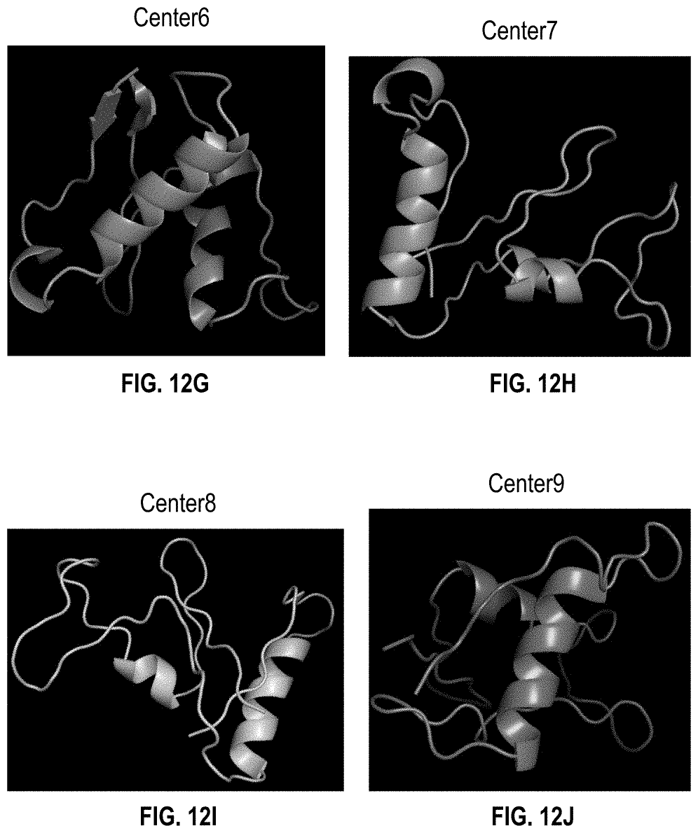

The scaffolds disclosed herein were analyzed with RaptorX to obtain their possible folding states. FIGS. 12A-12J illustrate the folding possibilities (center0 through center9 conformation shown in PyMOL) for Scaffold #1 having the amino acid sequence:

(SEQ ID NO: 72)
VIAWNSNNLDSKVGGNYNYLYRLFRKSNLKPFERDISTEIYQAGSTPCNG
VEGFNCYFPLQSYGFQPTNGVGYQPYRVV.

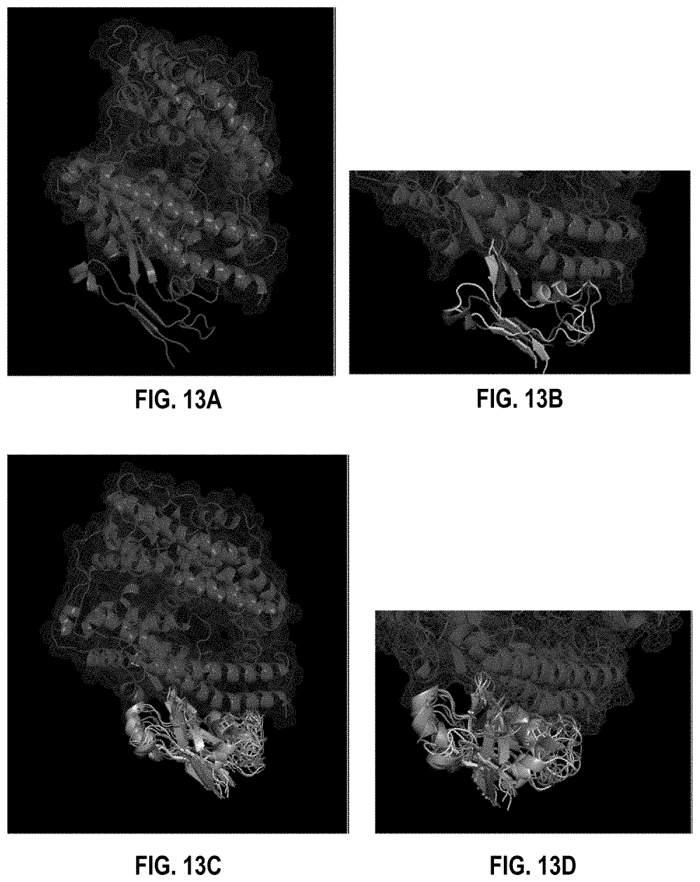

Each scaffold in its 10 possible folding states were overlaid with the CoV-2 RBD docked to ACE2 using PyMOL align commands to approximate binding. FIGS. 13A-13D illustrate various conformations of Scaffold #9 binding to ACE2. Scaffold #9 has the amino acid sequence:

((SEQ ID NO: 80)
EEVIAWNSNNLDSKVGGNYNYLYRCGSGSGQAGSTPGNGVEGFNGYFCLQ
SYGFQPTNGVGYQPYRVVRRR.

### Example 3. Design and Simulation of Synthetic Peptide Scaffolds Mimicking ACE2 Binding Domain

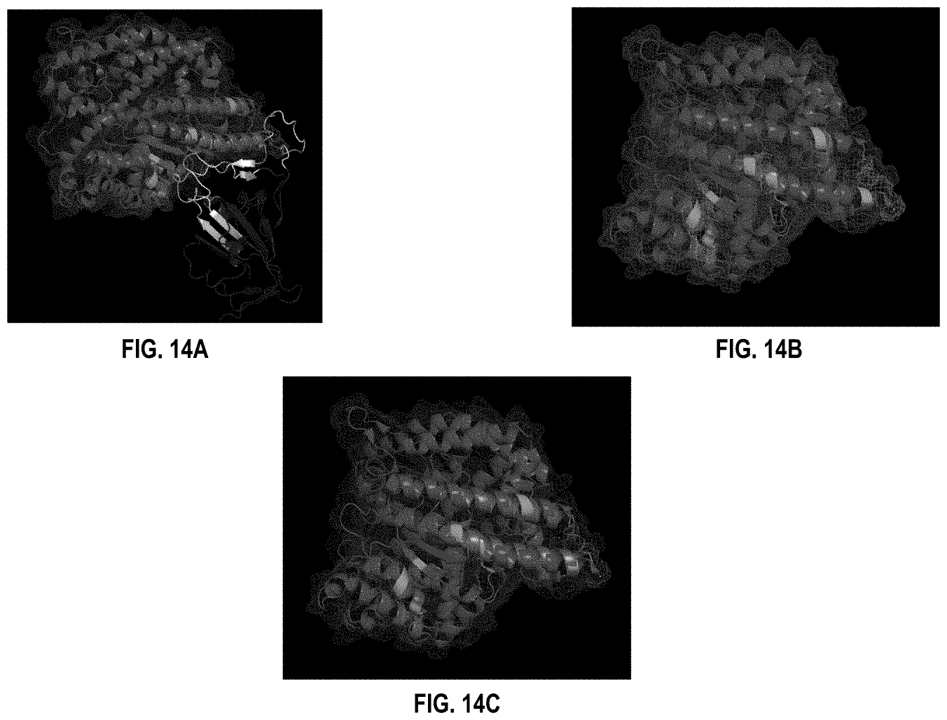

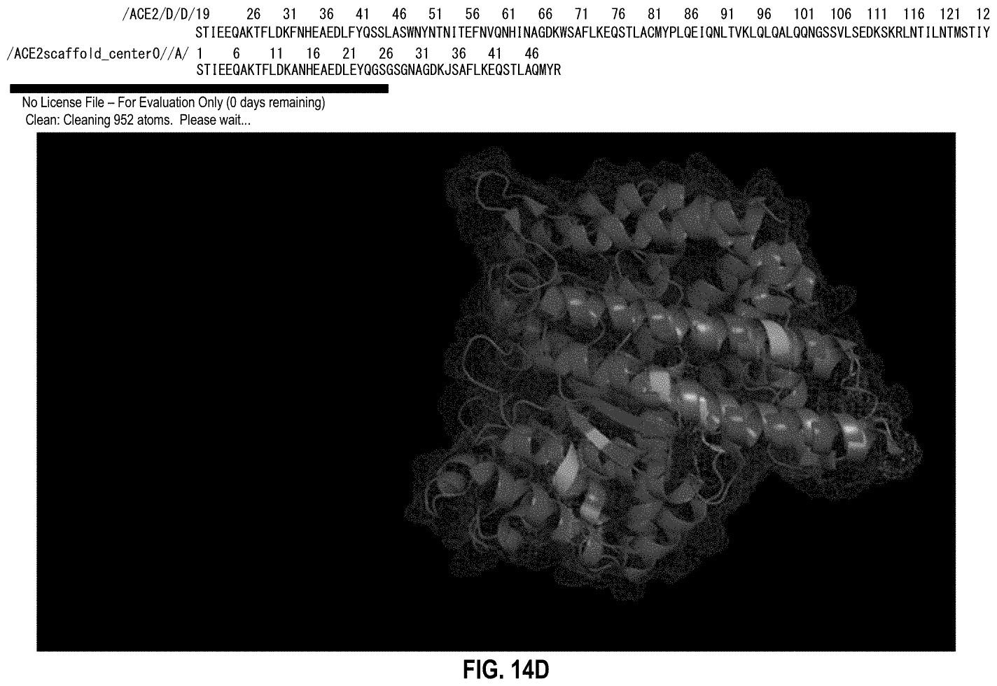

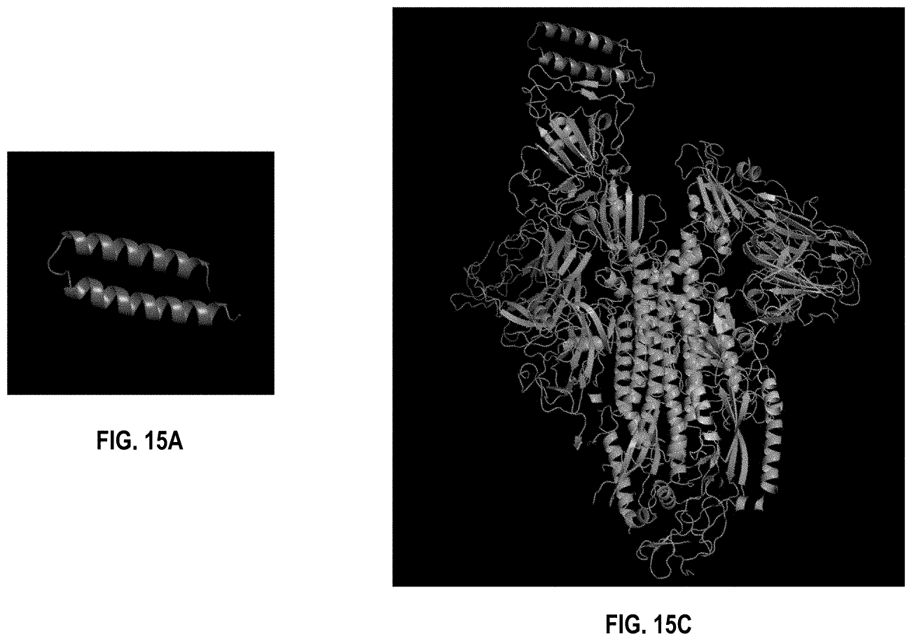

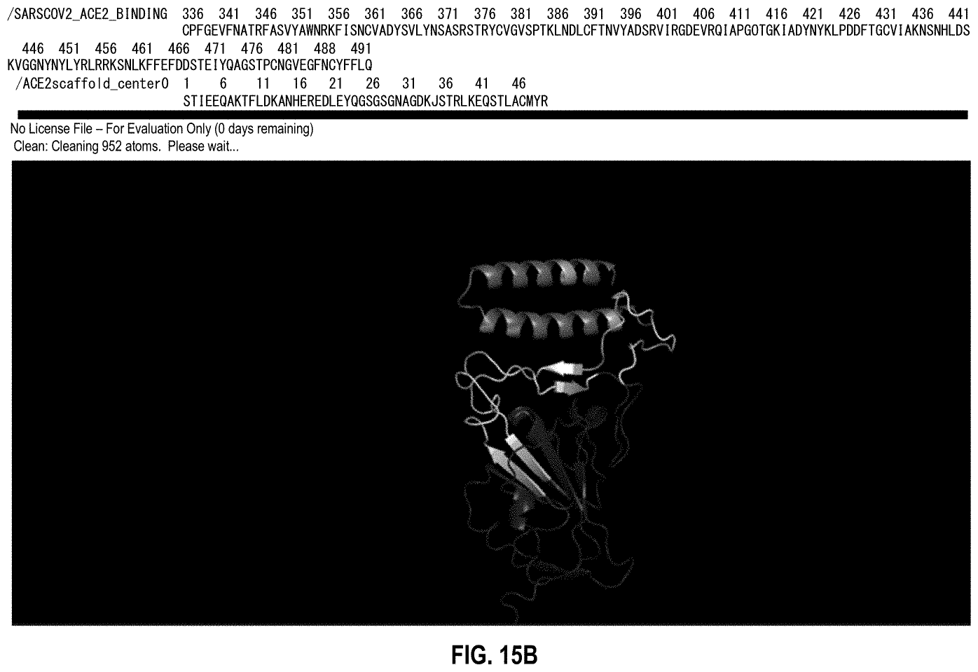

The binding interface of ACE2 was investigated in a similar way as disclosed in Example 2. A stretch of amino acid sequence from locations 19-84 of ACE2 (SEQ ID NO:140) appeared to be involved with binding to CoV-2 S protein. The critical binding residues include S19, Q24, D38, Q42, E75, Q76, and Y83, shown in green in FIGS. 14A-14D. Based on this analysis, ACE2 Scaffold 1 having the amino acid sequence STIEEQAKTFLDKFNHEAEDLFYQGSGSGNAGDKWSAFLKEQSTLAQMYP (SEQ ID NO:141) was synthesized (the GS linker is italicized and underlined). ACE2 Scaffold 1 appeared to have two critical binding motifs: STIEEQAKTFLDKFNHEAEDLFYQ (SEQ ID NO:149) (locations 19-42), and NAGDKWSAFLKEQSTLAQMYP (SEQ ID NO:150) (locations 64-84). FIG. 15A shows computer modeling of ACE2 Scaffold 1 truncated from the ACE2 protein, and FIGS. 15B-15C shows a simulation of ACE2 Scaffold 1 binding to CoV-2 S protein.

### Example 4. Comparison of Cryo-EM Structure and Simulated Structure

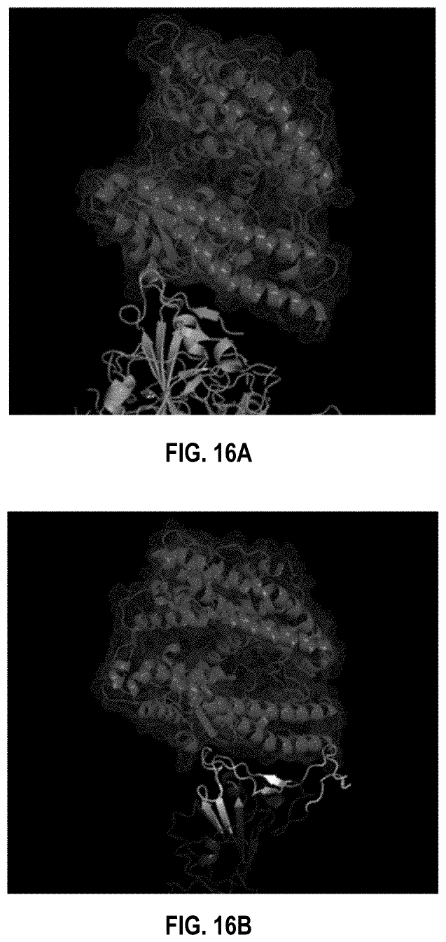

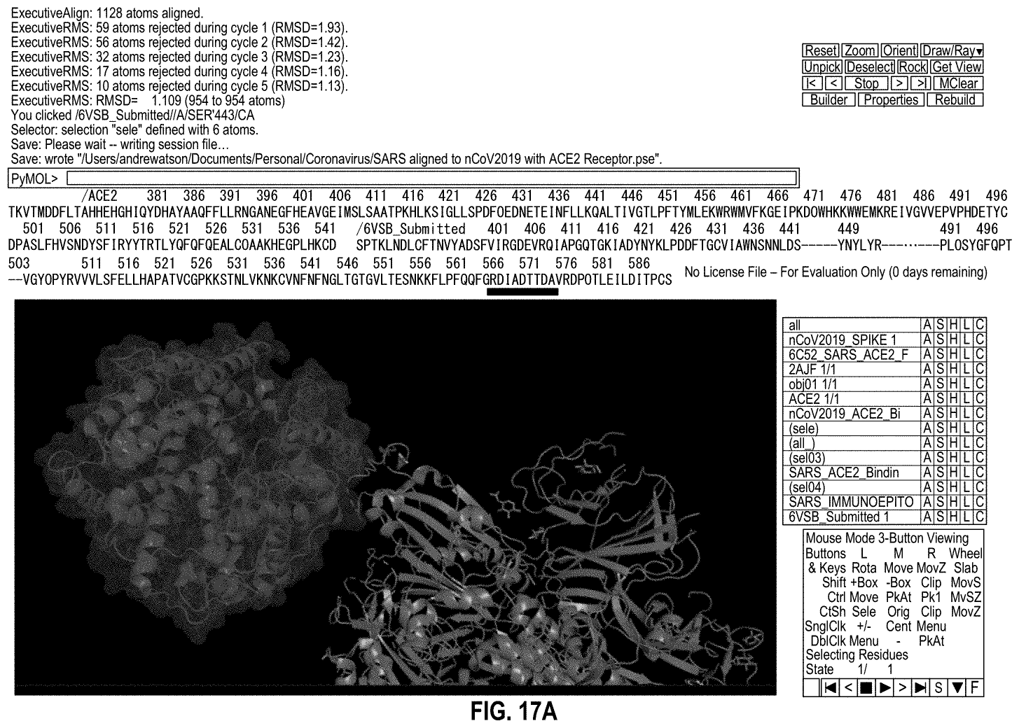

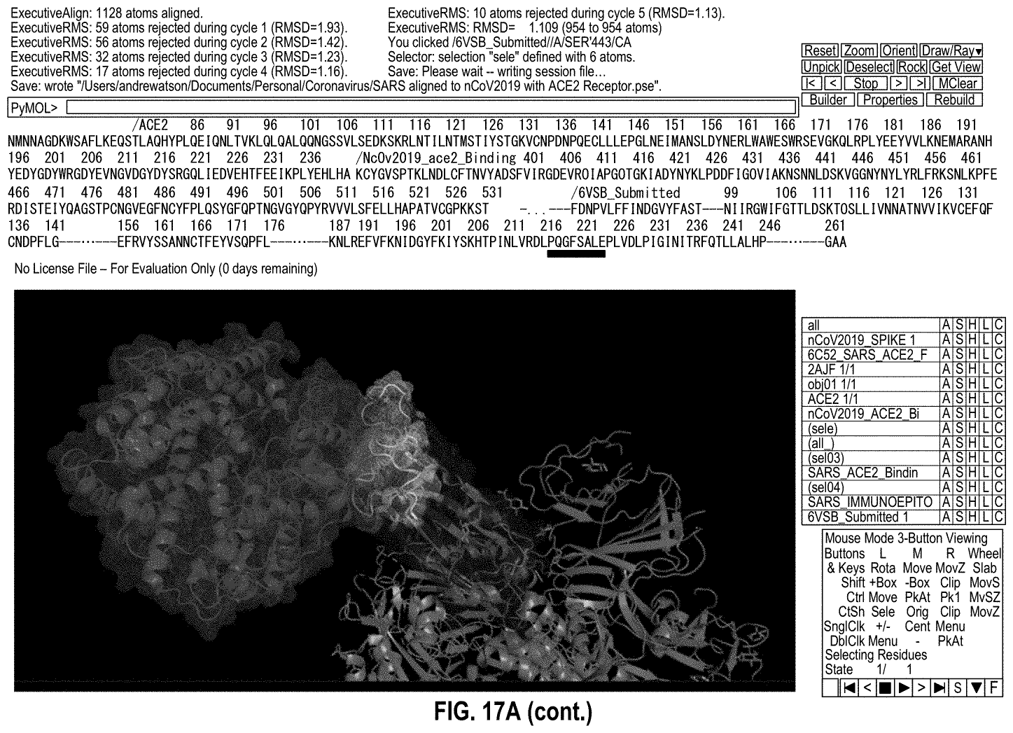

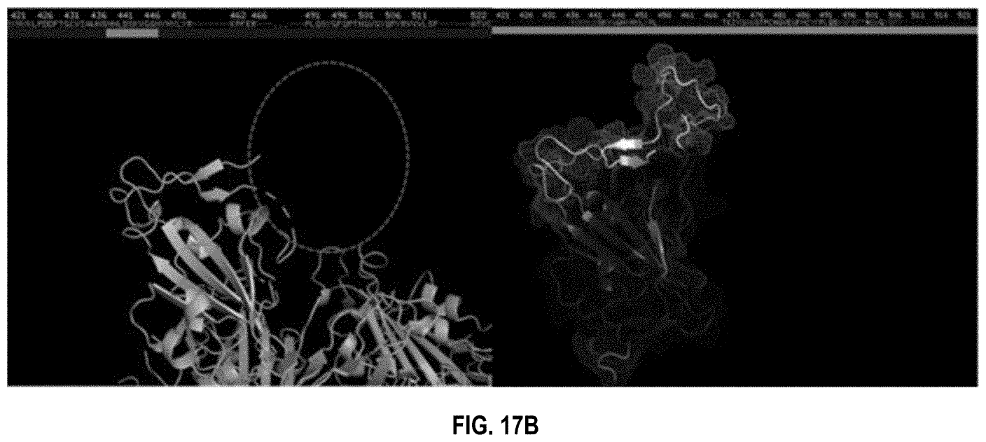

The computer simulated binding model disclosed in Example 1 was compared to the actual Cryo-EM solution structure of CoV-2 S protein determined and published by others (e.g., 22; Veesler Lab, Univ. of Washington (faculty.washington.edu/dveesler/publications/) (FIGS. 16A-16B). It was found that the binding interfaces were largely in agreement in terms of the stretches of the amino acid residues involved with binding with ACE2, with some discrepancies in the exact critical amino acids. Surprisingly, the cryo-EM structure published by others lacked these critical binding residues identified by computer modeling (FIGS. 17A-17B). The published structure did not contain residues 444-502, and therefore, lacked the critical binding motifs from locations 437 to 453 and from locations 473 to 507.

This example suggests that simulation of protein binding interfaces based on homologous binding scaffolds is an effective means to rapidly design binding scaffolds, inhibitors, and aid in drug discovery.

### Example 5. Producing CoV-2 Scaffolds

This example illustrates the design and production of CoV-2 scaffolds.

Simulation of SARS-CoV-2 S protein and determination of its ACE2-binding region. SWISS-MODEL was used to create a structural simulation of the CoV-2 virus in comparison to SARS-CoV-1 (PDB ID 6CS2). Next, PyMOL was used to align a truncated sequence of SARS-CoV-1 (locations 322-515) in its native conformation with the ACE2 receptor to SARS-CoV-2 (locations 336-531).

Mapping minimum interfacial sequences. Thermodynamic ΔG calculations of the simulated binding pocket of SARS-CoV-2 S protein with ACE2 were performed utilizing PDBePISA to determine the CoV-2 scaffold that binds to ACE2 and the critical binding residues in the scaffold.

Mapping immuno-epitopes. The entire sequence of the spike glycoprotein, as well as previously defined stretches of SARS-CoV-1 immunogenic sites were compared with similar sites on SARS-CoV-2. IEDB was used to recommend 2.22 antigenicity scoring to determine whether the homologous sites on SARS-CoV-2 were immunogenic.

Simulating truncated CoV-2 scaffolds. SWISS-MODEL and additional deep learning driven protein simulation approaches were used to perform structural simulations of the novel scaffolds. Various modifications were made to the scaffolds, such as adding linkers, replacing non-ACE2-binding and non-antibody-binding regions of the most proximal ACE2-interfacial fragment of the SARS-CoV-2 glycoprotein to incorporate antibody-binding regions. These domains can be variable and made in parallel to encompass a holistic screening of known and predicted immunogenic sites.

Peptide synthesis. Peptide scaffold sequences were designed and synthesized in-house or custom synthesized by third-party commercial providers such as sb-PEPTIDE (France). Mass spectrometry was used to confirm the appropriate peptide molecular weights.

In the case where targeting ligands were manufactured in-house, the method and materials were as follows. The peptides were synthesized using standard Fmoc-based solid-phase peptide synthesis (SPPS) utilizing a custom-built peptide robot, demonstrating about 120 second per amino acid coupling of a 9 amino acid sequence. Previously, 30-50 amino-acid peptides were synthesized in as little as 2 hours (FIG. 18). Synthesis may occur by any suitable means. For example, in the alternative to the peptide robot, yeast may be used to synthesize proteins. The peptides were synthesized on Rink-amide AM resin. Amino acid couplings were performed with O-(1H-6-Chlorobenzotriazole-1-yl)-1,1,3,3-tetramethyluronium hexafluorophosphate (HCTU) coupling reagent and N-methylmorpholine (NMM) in dimethyl formamide (DMF). Deprotection and cleavage of the peptides was performed with trifluoroacetic acid (TFA), tri-isopropyl silane (TIPS), and water. Crude peptide mixtures were purified by reverse-phase HPLC (RP-HPLC). Pure peptide fractions were frozen and lyophilized to yield purified peptides.

### Example 6. Cyclization of CoV-2 Scaffolds

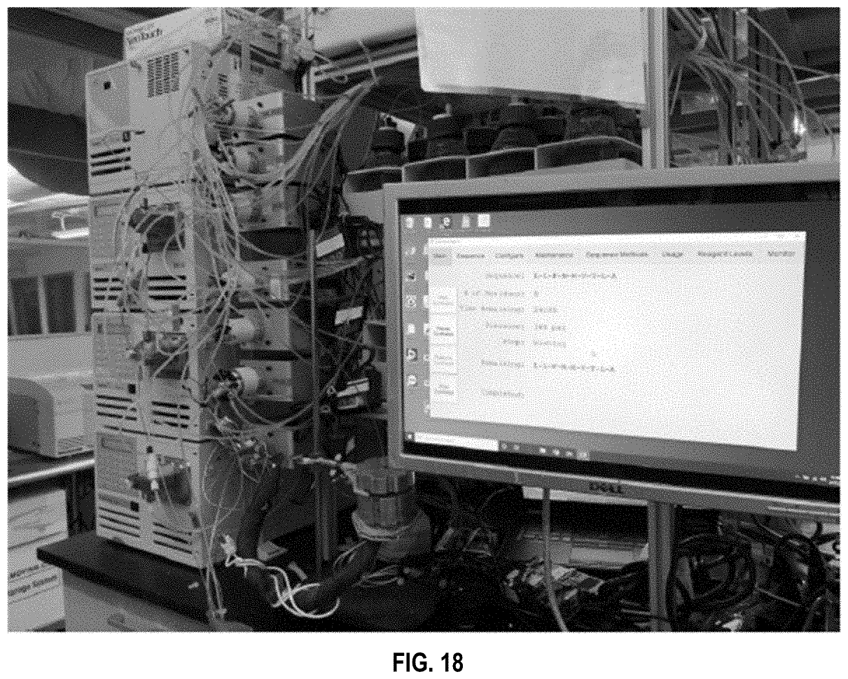

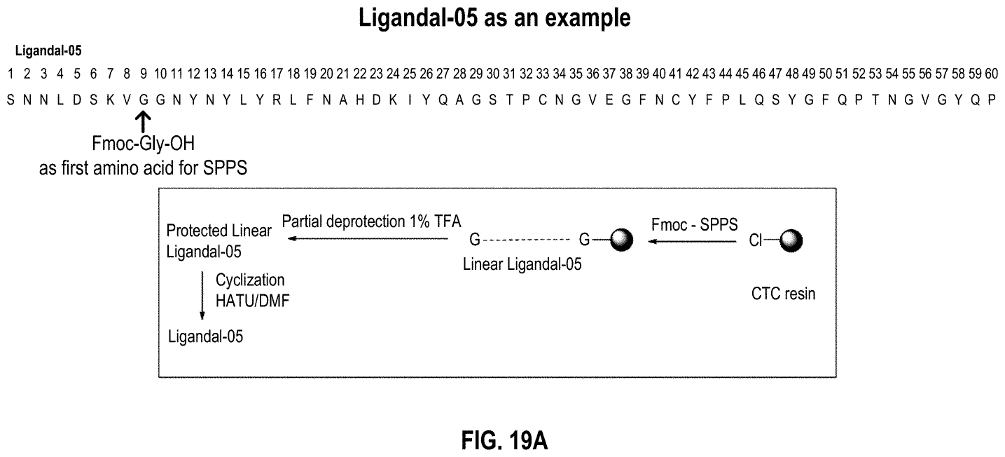

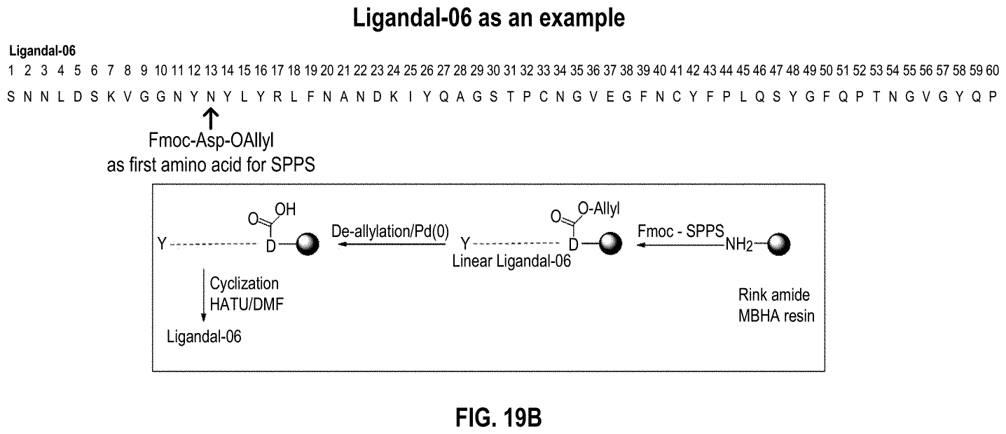

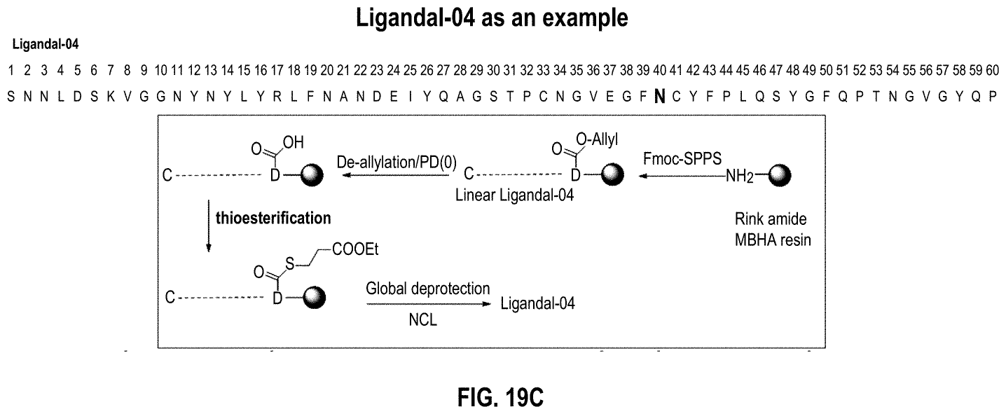

This example illustrates various strategies of head-to-tail cyclization of CoV-2 scaffolds, including: (1) head-to-tail cyclization of the side chain protected peptide in solution by amide coupling (FIG. 19A), (2) on resin head-to-tail cyclization by amide coupling (FIG. 19B), and (3) cyclization of purified linear thioester peptide by NCL (FIG. 19C). For Strategy (1), the synthesis was completed with an HPLC purity of ˜30% of the globally deprotected peptide. For Strategy (2), the synthesis was completed, and after de-allylation and global deprotection, the HPLC purity was ˜25%. For Strategy (3), the microwave synthesis was completed with ˜20% crude product obtained with O-Allyl protecting group. After de-allylation on resin cyclization was attempted with PyBOP/DIPEA for 16 hours. Desired product mass was not observed but thioesterification would be the next step.

### Example 7. Biolayer Interferometry of CoV-2 Scaffolds

Biolayer interferometry (“BLI”) directly interrogates binding between two or more analytes. This example demonstrates in vitro analysis of CoV-2 scaffolds using BLI to characterize binding dynamics by determining dissociation constants of the scaffolds associated with dimeric ACE2, and the inhibitory effects of the scaffolds on ACE2 to binding to the receptor binding domain (“RBD”). BLI was also used to determine the dissociation constants of the scaffolds associated with IgG neutralizing antibody (NAb).

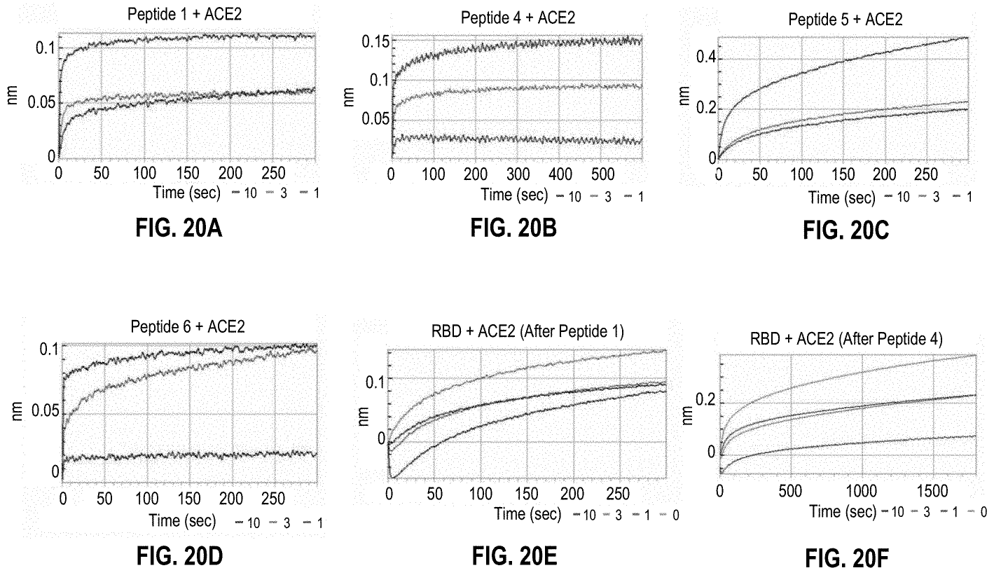

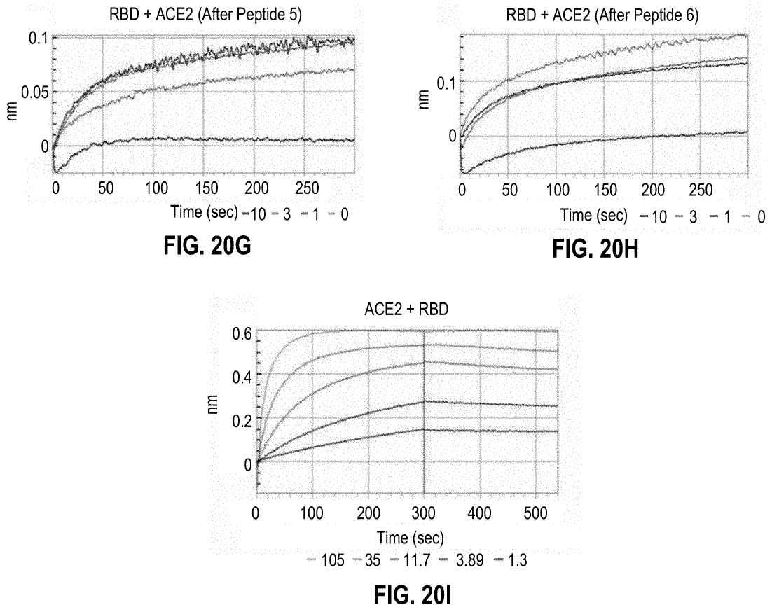

An Octet® RED384 biolayer interferometer (Fortebio) was used with sensor tips displaying anti-human IgG Fc (ACH), streptavidin (SA), nickel-charged tris-nitriloacetic acid (NTA), or anti-penta-his (HIS1K) in 96-well plates. For streptavidin tips, 1 mM biotin was used to block the surface after saturation with a given immobilized ligand. After protocol optimization with His-tagged versus biotin-tagged variants of ACE2 and RBD, the scaffold analytes in solution exhibited nonspecific binding to the sensor tip surface with NTA and HIS1K tips whereas biotinylated surfaces minimized this nonspecific binding. Furthermore, ACE2-His (Sino Biological) and RBD-his (Sino Biological) exhibited extremely weak binding to HIS1K tips. Therefore, dimeric-ACE 2-biotin (UCSF) and RBD-biotin (UCSF) were used on SA tips, and neutralizing monoclonal IgG antibody against the SARS-CoV-2 spike glycoprotein (CR3022, antibodies-online) were used on AHC tips for all studies. Nonspecific binding was still observed with Scaffold #8 (SEQ ID NO:79) binding to a neutralizing antibody on AHC tips, which complicated efforts to determine the K_{D} using Scaffold #8 as the analyte compared to the neutralizing antibody ligand. All stock solutions were prepared in a 1×PBS containing 0.2% BSA and 0.02% Tween20. The following ligands and analytes were studied:
1) Dimeric ACE2-biotin was immobilized on SA tips (˜2.5 nm capture).
   a. Scaffold #4 (“Peptide 1,” SEQ ID NO:75), Scaffold #7 (“Peptide 4,” SEQ ID NO:78), Scaffold #8 (“Peptide 5,” SEQ ID NO:79), and Scaffold #9 (“Peptide 6,” SEQ ID NO:80) were introduced to immobilize ACE2 in concentrations of 1, 3 and 10 μM (FIGS. 20A to 20D).
   b. Sensor tips were removed from peptide solutions and introduced to 35 μM RBD-His (Sino Biological) (FIGS. 20E to 20H).
2) RBD-biotin was immobilized on SA tips (˜5 nm capture).
   a. ACE2-His (Sino Biological) was introduced to immobilized RBD at 1.3, 3.9, 11.7, 35, and 105 μM concentrations (FIG. 20I).
3) Neutralizing IgG antibody was immobilized on AHC tips (˜1 nm capture).
   a. Scaffold #4, Scaffold #7, Scaffold #8, and Scaffold #9 were introduced to immobilized ACE2 at 0.37, 1.1, 3.33, and 10 μM concentrations (FIG. 20A to 20D).
   b. RBD-His (Sino Biological) was introduced to immobilized neutralizing antibody (CR3022, antibodies-online) at 1, 3, 9, 27, and 81 μM concentrations.
   c. 117 nM RBD-His (Sino Biological) was mixed with ACE2-His at 0 (RBD only), 2.88, 8.63, 25.9, and 77.7 μM concentrations. Next, immobilized neutralizing antibody (CR3022, antibodies-online) was introduced.

BLI was used to determine dissociation constants of selected scaffolds associated with dimeric ACE2 and the inhibitory effects of the scaffolds on ACE2 binding to RBD. As demonstrated in FIGS. 20A-20I, the CoV-2 scaffolds tested in this experiment prevented ACE2 from binding to S protein RBD in a concentration-dependent manner. All scaffolds tested exhibited potent inhibition of RBD binding to ACE2 at 10 μM concentrations. The scaffolds were associated with ACE2 at 1, 3 and 10 μM concentrations until saturation (FIGS. 20A-20D). After binding to ACE2, the ACE2 association of SARS-CoV-2 RBD at 35 μM was measured in the absence of scaffolds (FIGS. 20E-20H), and scaffolds were shown to act as strong antagonists even after a saline+FBS wash. Interestingly, association of ACE2 with Scaffold #8 at 1 μM and 3 μM enhanced RBD binding, while 10 μM concentrations strongly abrogated binding (FIG. 20G). All other peptides exhibited a dose-response-like behavior in preventing RBD binding, including at 1 μM and 3 μM concentrations (FIGS. 20E, 20G, and 20H). To assess competitive irreversible antagonism, the scaffolds were not included within the final solution of 35 μM RBD as depicted in FIG. 20I.

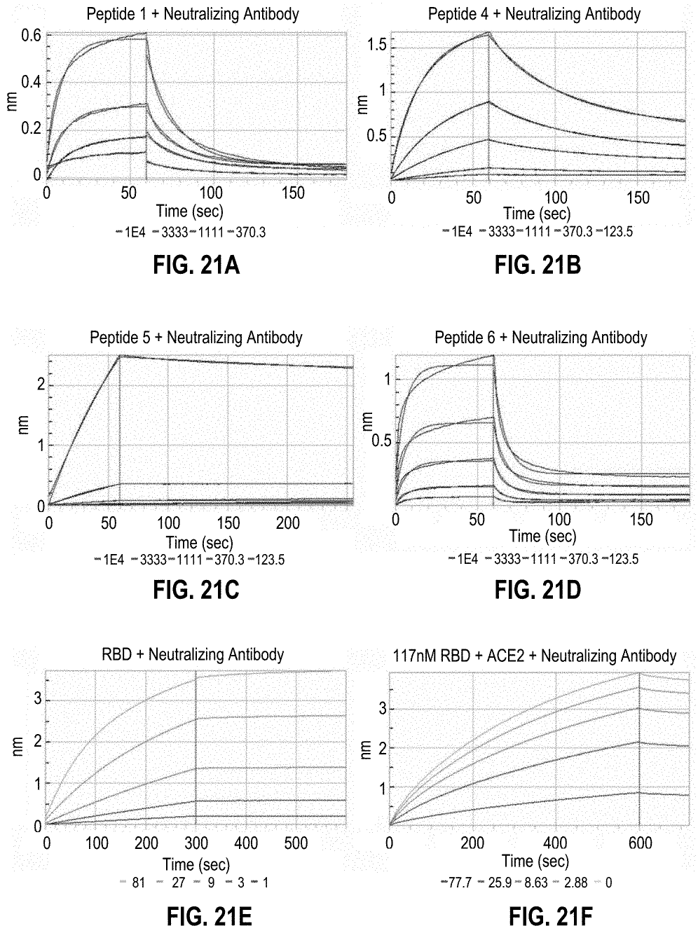

BLI was also used to determine dissociation constants of selected scaffolds associated with an IgG neutralizing antibody. Scaffold #8 exhibited nonspecific binding to the sensor tip (FIG. 21C), preventing determination of KD against the neutralizing antibody. This nonspecific binding with Scaffold #8 was observed in all studies that did not utilize biotinylated substrates with biotin blocking of the sensor surface. However, single-micromolar binding affinities for all other scaffolds were determined with the neutralizing antibody (FIGS. 21A, 21B, and 21D). Next, the dissociation constant for increasing concentrations of RBD with anti-RBD neutralizing antibody was measured (FIG. 21E). To examine ACE2's inhibition of neutralizing antibody binding to the RBD, 117 nM RBD was mixed with increasing concentrations of ACE2 prior to introduction to immobilized neutralizing antibody (FIG. 21F). The half-maximal inhibitory concentration (IC50) of ACE2 inhibiting interaction between RBD and the neutralizing antibody was interpolated to be about 30-35 nM for ACE2 when the RBD concentration was 117 nM (FIG. 21F). These data indicate that ACE2 binds more potently to the RBD than the neutralizing antibody does, and that soluble ACE2 can act as a potent “cloak” against neutralizing antibody recognition even at fractional molarities to SARS-CoV-2 spike RBDs. All scaffolds tested in this experiment were immunogenic.

Using the BLI data presented in the Figures above, the dissociation constants (K_{D}) and RMax values (steady-state binding analyses) were determined for 1) Scaffold #4, Scaffold #7, Scaffold #8, and Scaffold #9 binding to ACE2, 2) ACE2 and neutralizing antibodies binding to RBD, 3) scaffolds' binding to neutralizing antibodies, and 4) RBD binding to neutralizing antibodies in the presence of increasing concentrations of ACE2. The K_{D} and RMax values are presented in Table 2 below.

TABLE 2

| Binding Partners | RMax | K_{D} |
|---|---|---|
| Scaffold #4 | 0.12111318 ± 0.0239083 | 1.80E−06 ± 1.1E−06M |
| Binding to ACE2 | | |
| Scaffold #7 | 0.22716674 ± 0.0339753 | 5.20E−06 ± 1.7E−06M |
| Binding to ACE2 | | |
| Scaffold #8 | 0.57363623 ± 0.1333544 | 4.00E−06 ± 2.2E−06M |
| Binding to ACE2 | | |
| Scaffold #9 | 0.13006174 ± 0.032393 | 2.40E−06 ± 1.7E−06M |
| Binding to ACE2 | | |
| Scaffold #4 | 0.71962807 ± 0.0759471 | 4.30E−06 ± 1.0E−06M |
| Binding to NAb | | |
| Scaffold #7 | 0.22716674 ± 0.0339753 | 5.20E−06 ± 1.7E−06M |
| Binding to NAb | | |
| Scaffold #8 | ∞ | N/A |
| Binding to Nab | | |
| Scaffold #9 | 1.18656192 ± 0.0552815 | 3.30E−06 ± 3.9E−07M |
| Binding to NAb | | |
| ACE2 Binding | 0.57847430 ± 0.0155693 | 2.30E−09 ± 3.0E−10M |
| to RBD | | |
| NAb Binding | 4.40220793 ± 0.159029 | 8.60E−09 ± 1.1E−09M |
| to RBD | | |

The data presented in Table 2 demonstrates that Scaffold #s 4, 7, 8 and 9 have dissociation constants of 1.8±1.1 μM (Scaffold #4), 5.2±1.7 μM (Scaffold #7), 2.4±1.7 μM (Scaffold #8), and 2.4±1.7 μM (Scaffold #9) with ACE2, and 4.3±1.0 μM (Scaffold #4), 5.2±1.7 μM (Scaffold #7), unknown (Scaffold #8), and 3.3±1.19 μM (Scaffold #9) with a neutralizing antibody, respectively. Scaffold #8 binding to the neutralizing antibody was undetermined due to a technical error caused by nonspecific interactions with the sensor tip. The dissociation constant of ACE2 with RBD is 2.3±0.3 nM, while the dissociation constant of the neutralizing antibody with RBD is 8.6±1.1 nM. These data indicate that Scaffold #4 exhibited the strongest affinity for both the neutralizing antibody and ACE2.

### Example 8. Infection of ACE2-HEK293 Cells with SARS-CoV-2 Spike Protein Pseudotyped Lentivirus

ACE2-HEK293 cells (BPS Bioscience) were transduced with pseudotyped lentivirions displaying the SARS-CoV-2 spike glycoprotein (BPS Bioscience) and assessed for luciferase activity and trypan blue toxicity 60 hours post-infection. A neutralizing monoclonal IgG antibody against SARS-CoV-2 spike glycoprotein (CR3022, antibodies-online), ACE2 (Sino Biological), receptor-binding domain (RBD) of spike glycoprotein (Sino Biological), and selected scaffolds of the present disclosure were used as inhibitors of infection. Infection was quantitated via bioluminescence, and toxicity was characterized via a trypan blue absorbance assay utilizing a Synergy™ H1 BioTek spectrophotometer.

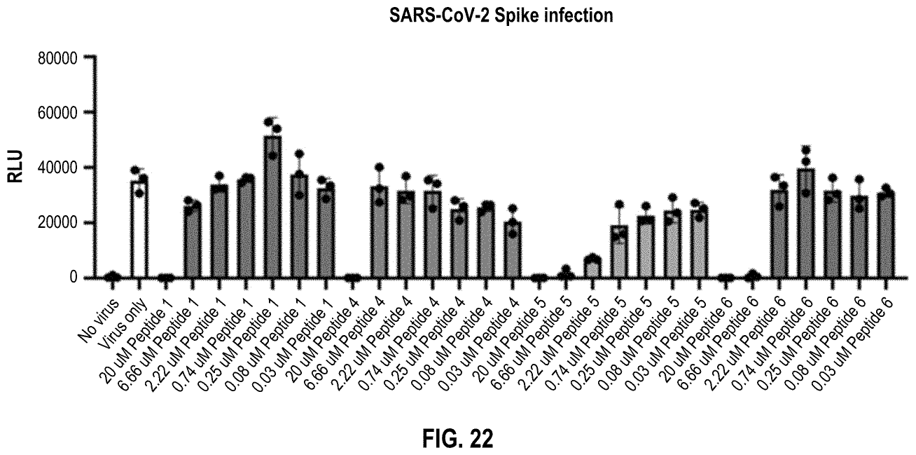

As shown FIG. 22, Scaffold #4 and Scaffold #7 did not block SARS-CoV-2 spike protein pseudotyped virus infection of ACE2-HEK293 cells at concentrations below 20 μM, as assessed by luciferase activity 60 hours post-infection. Yet, Scaffold #8 and Scaffold #9 both impeded viral infection at 6.66 μM, with Scaffold #8 significantly exhibiting this blocking effect in the nanomolar range (80 nM and 30 nM, p<0.05, t-test comparison with virus only). (*, p<0.05; ***, p<0.001; unpaired student's t-test, technical triplicates).

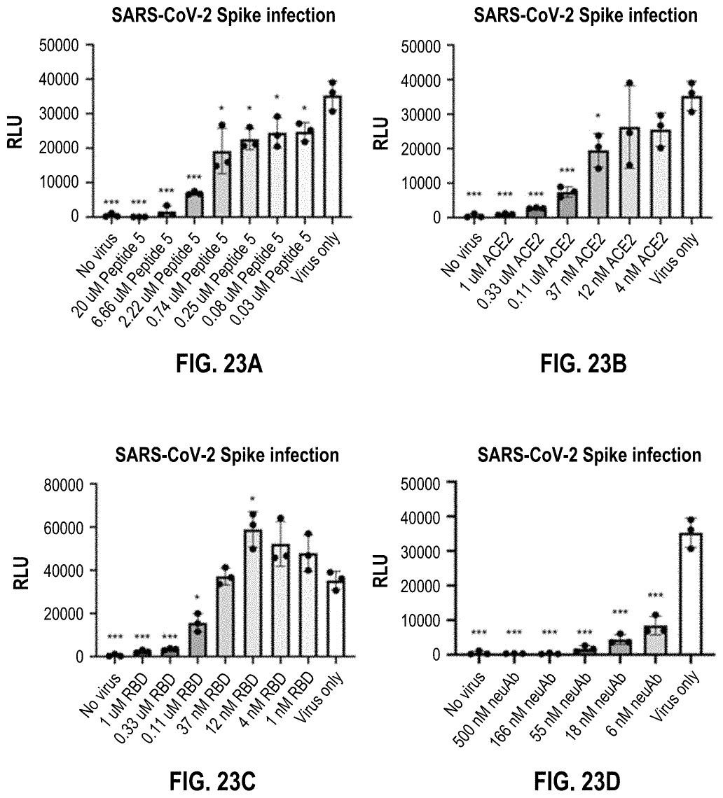

FIGS. 23A-23D show a virtually complete inhibition of SARS-CoV-2 spike protein pseudotyped virus infection by soluble RBD and soluble ACE2 at 0.33 uM, while a SARS-CoV-2 neutralizing antibody inhibited infection to a similar extent at concentration as low as 6 nM. Intriguingly, 12 nM RBD enhanced infection. (*, p<0.05; ***, p<0.001; unpaired student's t-test, technical triplicates).

Importantly, with the exceptions of 20 μM dose of Scaffold #8 causing cell death and leading to visible aggregation of the scaffold in solution and 166 nM neutralizing antibody enhancing cell survival, the addition of the scaffolds, soluble ACE2, soluble RBD, or SARS-CoV-2 neutralizing antibody at different concentrations did not result in statistically significant changes in cell viability in the presence of virus, ca. 50%.

Accordingly, the novel synthetic peptide scaffolds disclosed herein have been demonstrated to block a virus from associating with cells, while also demonstrating epitopes for antibody and T cell receptor formation. This experiment demonstrates that the tested scaffolds effectively blocked >95% of infection of pseudotyped lentiviruses displaying the SARS-CoV-2 spike protein infecting ACE2-expressing cells without toxicity at EC95 dose, and that the tested scaffolds prevented association of the SARS-CoV-2 receptor binding domain (RBD) with ACE2 even at extremely high RBD concentrations (35 μM). The tested scaffolds exhibited an IC50 in sub-micromolar range with statistically significant viral inhibition at 30 nM.

### Example 9. Effects of CoV-2 Scaffolds in Live Virus

The inhibitory effects of Scaffold #4, Scaffold #7, Scaffold #8, and Scaffold #9 were tested in live virus in CaCo2 cells, followed by toxicity test. See Table 3 for the antiviral activity of the tested scaffolds against SARS-CoV-2 shown as 50% cell culture infectious dose (CCID50) determined by endpoint dilution on Vero 76 cells, and the percent toxicity of the tested scaffolds determined by neutral red dye uptake on Caco-2 cells.

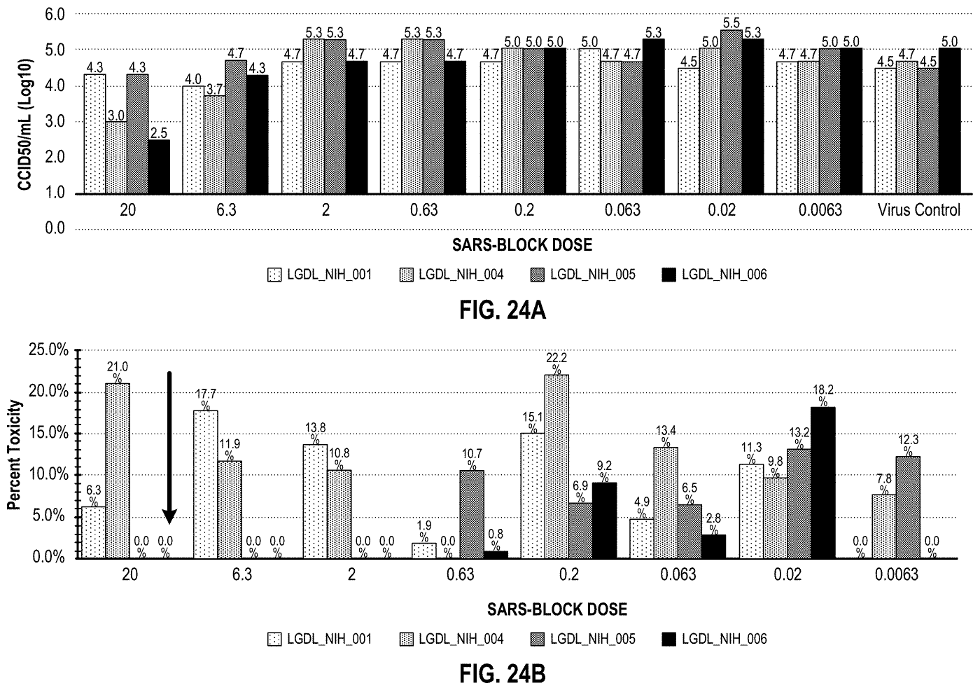

FIG. 24A shows that the tested scaffolds exhibited over 90% inhibition of viral load (EC90) in live virus at micromolar concentrations. FIG. 24B shows no toxicity with up to 2.5 log inhibition of viral load with live SARS-CoV-2 virus.

### Example 10. Molecular Dynamics and Modeling of Scaffolds

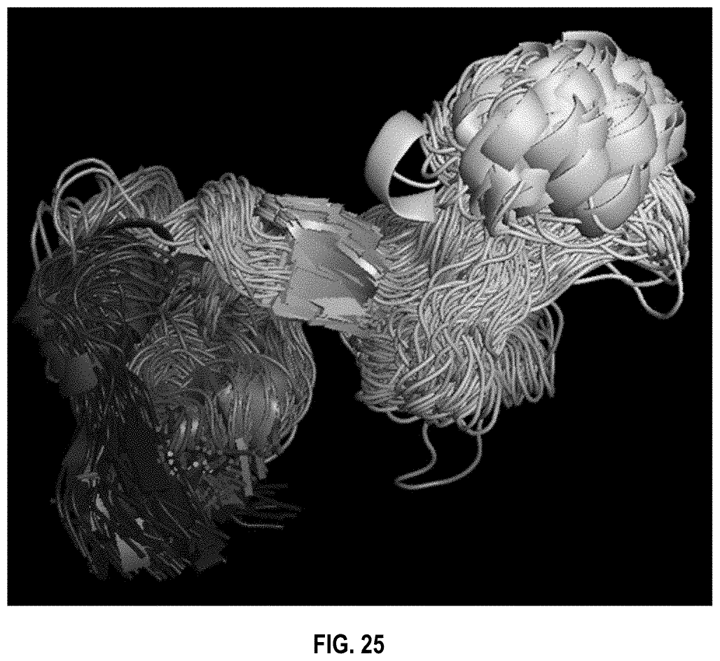

As shown in FIG. 25, molecular dynamics modeling was used to model the folding of Scaffold #4 having the amino acid sequence VIAWNSNNLDSKVGGNYNYLYRCFRKSNLKPFERDISTEIYQAGSTPGNGVEGFNGYFCL QSYGFQPTNGVGYQPYRVV (SEQ ID NO:75). It was investigated whether the best-scoring structure was stable by itself in solution, and how flexible it would be. Peptides can sometimes refold very quickly; if the starting point is not near a local minimum that would be evident from modeling. Whether the peptide is stable at short to intermediate time scales is a much easier question than whether that is the global minimum.

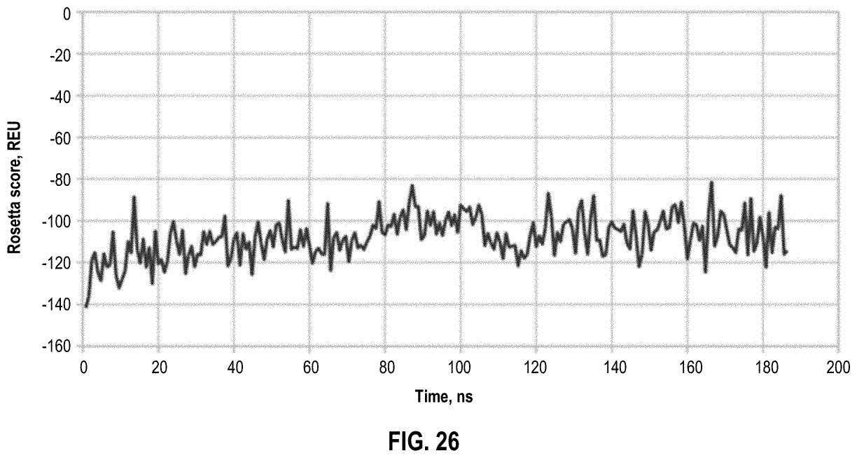

Qualitatively, the big loop (RKSNLKPFERDISTE; SEQ ID NO:128) folded under and up until it touched the 13 sheet in the middle, <1 ns; the TNG binding loop folded down and towards the middle, about 20 ns; then the structure was basically stable for the rest of the simulation but remained very flexible. The binding loops were highly flexible—they unfolded and refolded constantly, the motion was 5-6 A rmsd; and the middle of the structure was fairly rigid, <1 A rmsd. Rosetta scores during folding are shown in FIG. 26. These data indicate that about 20 units were lost relative to the idealized structure. Rosetta has a not-entirely physical energy function which is optimized for well-ordered proteins with stable folds but does not perfectly model solvent effects, which drive the loop to fold under. It is possible that there are not particularly good specific binding interactions and that the analysis has trouble with disordered regions. Annealing structures from near the end of the run carefully may be able to find even better Rosetta scores.

All scaffold structures can be run through the above-disclosed steps in replicas. Improvements can be made to sample longer time scales efficiently. For example, AMD can give 100×effective speedup vs plain MD, such that even folding from scratch or tracing binding pathways can be analyzed.

In designing peptides, rigidity may be the most important consideration. Crosslinking chains, either by H-bond or by covalent link (e.g., stapled peptides), can increase the effective concentration of peptides in a ready-to-bind conformation, and reduce the likelihood of unbinding of a peptide due to flexing. There is probably strong selection pressure to make the biological designs more flexible, especially in surface-exposed regions. The flexibility is taken into consideration in subsequent designs, for example, by adding multiple prolines, or determining how to make the two β sheet bits into one bigger β sheet. Some exemplary peptides including Scaffold #s 4, 5, 6, 7, 8, and 9 (SEQ ID NOs:75-80, respectively) were used in further structural analysis and modification.

The sequence or partial sequence of the scaffolds was tested initially without the receptor binding domain (RBD) to determine whether it produces expected structure. An initial test can be performed using the sequence CKMSECVLGQSKRVQALLFNKVTLAGFNGYFC (SEQ ID NO:129), which is the loop only from Scaffold #8, with cysteine residues at the N-terminus and C-terminus to ensure closure; and the partial sequence of KMSECVLGQSKRVDFC (SEQ ID NO:70), which is slightly larger than the immuno-epitope by adding three amino acid residues to the C-terminus (bold and underlined), also looped.

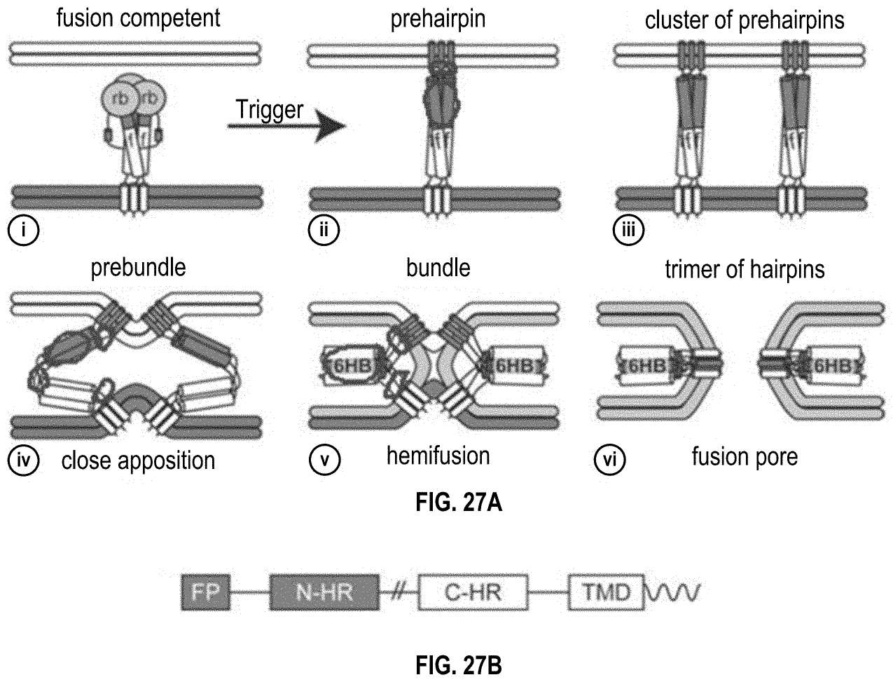

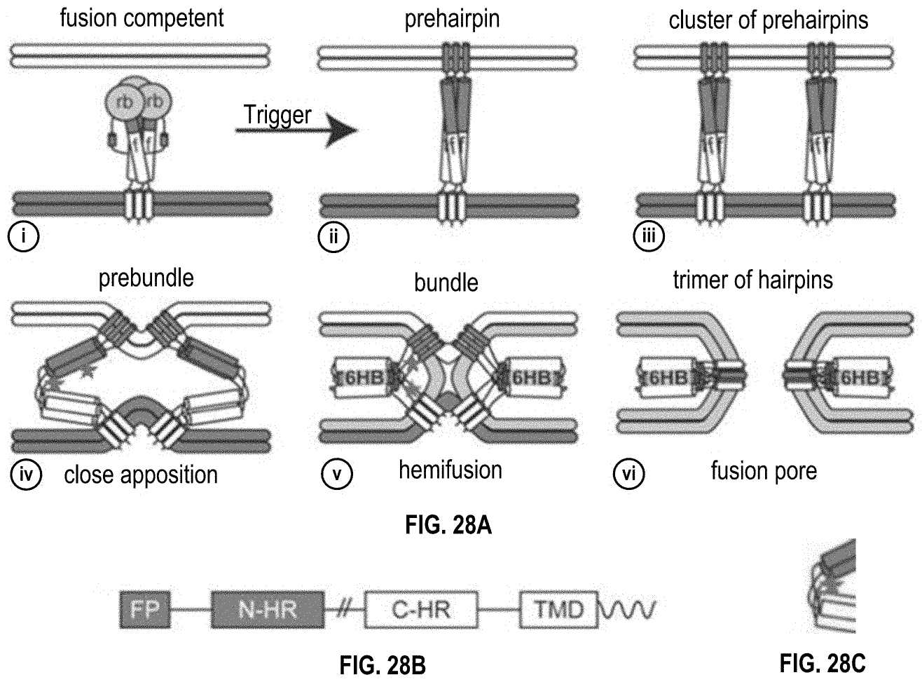

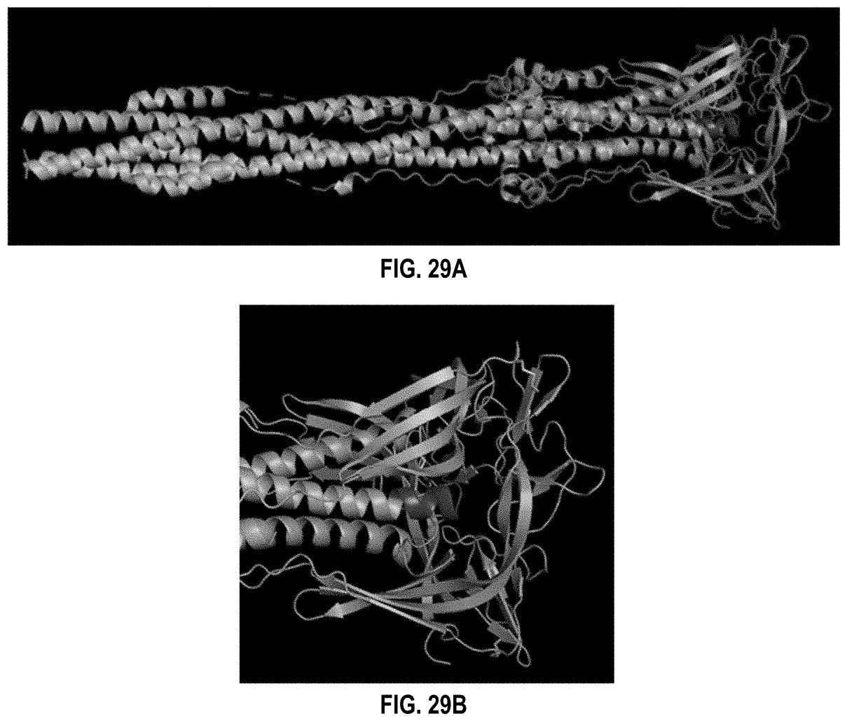

As shown in FIG. 27, the unique epitopes on S protein that are only exposed during fusion were examined. The binding sites which would prevent the process from moving to the next step of neutralizing were also examined and some hidden epitopes were exposed indefinitely (FIG. 28). The sequence 6XRA from Protein Data Bank (PDB, distinct conformational shapes of SARS-CoV-2 spike protein, www.rcsb.org/structure/6XRA), which is the bundle configuration of the S protein during fusion, was investigated. The sequence of KMSECVLGQSKRV (SEQ ID NO:71) was fitted to the protein structure as depicted in FIG. 29A and shown enlarged in FIG. 29B. It was determined that this was exactly the location of one of the binding sites identified in FIGS. 27-28 located in the hinge between HR1 and HR2 during the pre-bundle stage, i.e., the binding site enlarged in FIG. 29B. Thus, it was predicted that Scaffold #8 prevented fusion/infection with pseudo-typed virus at nanomolar concentrations because some of it bound at this site, using a mechanism completely independent of ACE2. This hypothesis is supported by the determination that the binding of Scaffold #8 to ACE2 was not much better than for the other peptides; but the effects at very low (nM) concentrations were notably different, which suggests a second mechanism of action. The second mechanism of action only kicks in with actual spike protein and actual virus. Therefore, it is probably binding to the spike protein. This binding pocket is surrounded by the other two chains in the bundle. Any peptide that manages to get in the binding pocket would likely have an ultra-tight binding, maybe at a concentration of single digit nanomolar or less and it would also completely disrupt fusion. Additional analysis is required to determine the series of rearrangements the spike protein goes through to go from its original folded up form to the bound form. There may be multiple pathways, and only some of them may have this site temporarily open during binding. Also, there are many other binding sites where a small fragment of the 6XRA structure may compete with the whole structure. An in silico or in vitro screen of every 10-20 amino acids linear fragments may find more sites.

While Scaffold #8 itself is unlikely to be optimal because the RBD bits do not appear to do anything here other than provide bulk or steric hindrance, which probably makes binding less tight, although also disrupts the hairpin structure more, it serves as proof of concept. The sequence of KMSECVLGQSKRVDFC (SEQ ID NO:70) having a disulfide bond was tested initially and subsequently optimized.

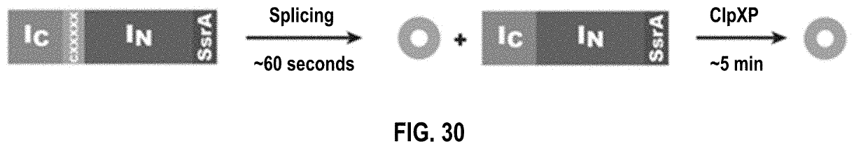

The genetically encoded cyclic peptides, self-catalyzing as described previously, were also utilized (16). An extein of choice is inserted in the region identified in FIG. 30 with a Cys or Ser residue in position 1, which is necessary for intein splicing). An example of the sequence is as follows: HHHHHHGENLYFKLQAMGMIKIATRKYLGKQNVYDIGVERYHNFALKNGFIASNCAAAAA CLSYDTEILTVEYGILPIGKIVEKRIECTVYSVDNNGNIYTQPVAQWHDRGEQEVFEYCLED GCLIRATKDHKFMTVDGQMMPIDEIFERELDLMRVDNLPNGTAANDENYALA (SEQ ID NO:152). The bold sequence is the resulting cyclic peptide, the rest splices itself out—cyclic 6 amino acids or longer, and the first amino acid can be Cys or Ser. Intein-extein fusion can be used as a mechanism for fusing a peptide or recombinant/synthetic sequence with a self-catalyzing and self-spliced out sequence to create fusion between two peptide sequences.

### Example 11. Further Optimization of Scaffold Sequences

Additional sequences for designing the scaffold were identified based on consensus of the highest scores, and the scores were a combination of stability and binding affinity, with heavy emphasis on the affinity.

For a peptide to act as a “super binder” having very high affinity, it is desirable to have a longer loop that sticks out on the ACE2 side and makes more contacts with it. The goal is to improve stability of the scaffold by itself without compromising binding affinity. Preferably, binding affinity is improved by nudging the same binding residues into better positions.

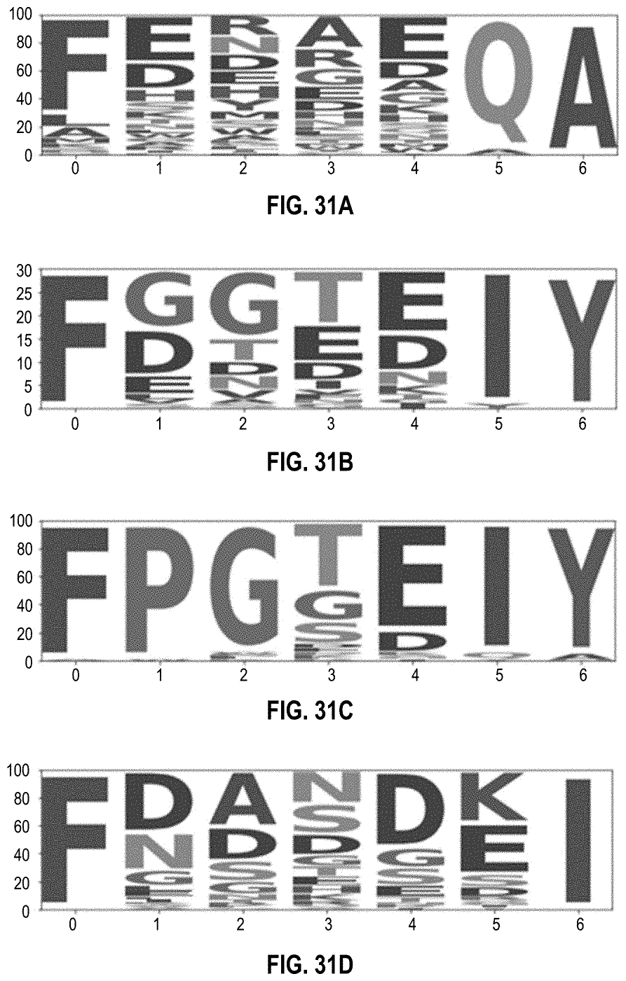

FIGS. 31A-31D illustrate how to screen and optimize the peptide sequence. Step 1: Length 5 loop in RLxxxxxQA, about 60k tries. F at the first position was most prevalent. FIG. 31A. Step 2: Fix the first F—at this point -YQA seemed to be closer to the next residue than -QA, and therefore, a length 5 loop RLFxxxxxYQA was built, about 15k tries. Very nicely, this reproduced the native -TEIYQA bit; the single best sequence from that run was the RLFDGTEIYQA. FIG. 31B. Step 3: The geometry of the second residue was not too incompatible with proline, but the loop builder algorithm had trouble inserting that; tried fixing a proline in that position. Build length 4 loop with RLFPxxxxYQA, about 22k tries. The loop sequence including RLFPGTEIYQA scored equal to RLFDGTEIYQA. The loop sequence including RLFPGTDIYQA was good as well. FIG. 31C. Step 4. Build a slightly longer loop, 5 residues with RLFxxxxxIYQA, about 41k tries. This run was less conclusive. The scoring function favored D or E at every position. This may be because they can form hydrogen bond to their own backbone when the loop is facing out into the solvent. Additionally, they did not seem to be necessarily interacting with non-adjacent residues, but they may still stabilize the loop. The best candidate from this batch was RLFNANDKIYQA or RLFNANDEIYQA. FIG. 31D.

### Example 12. Use of Scaffold for siRNA Delivery

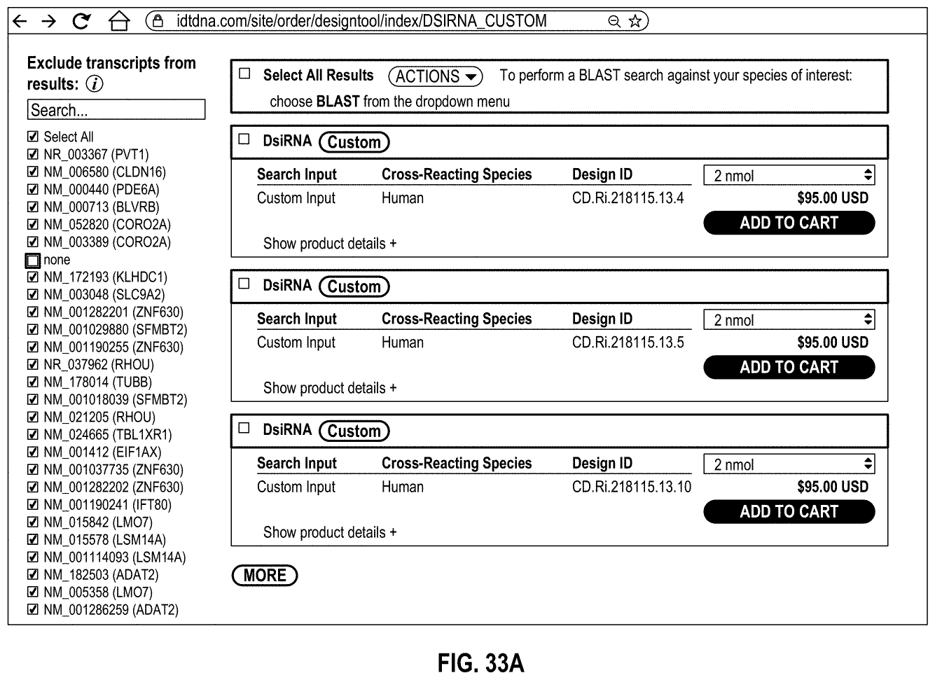

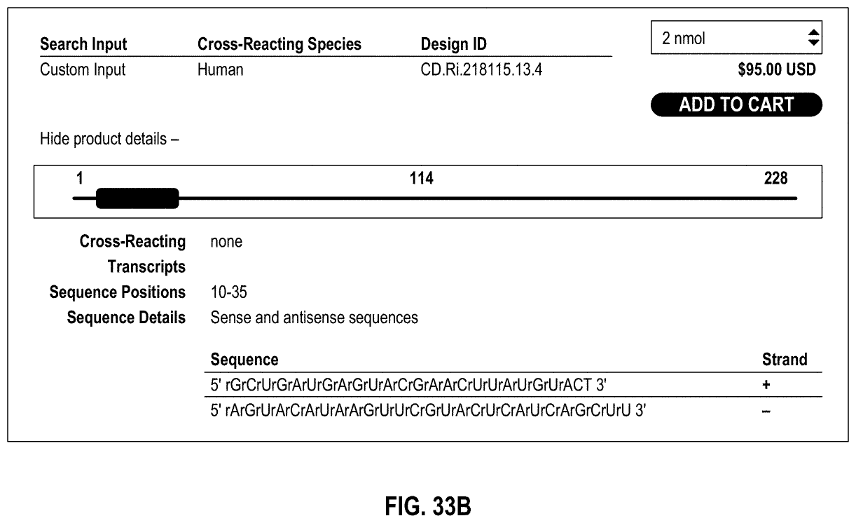

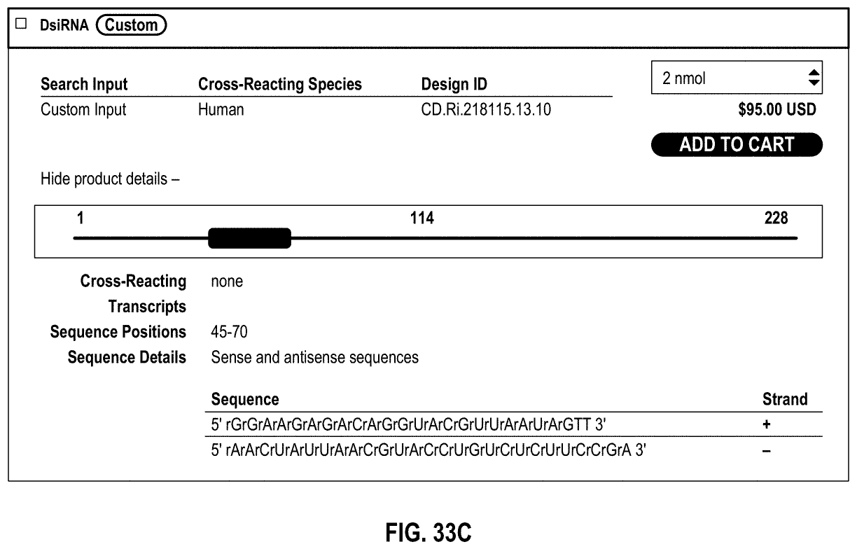

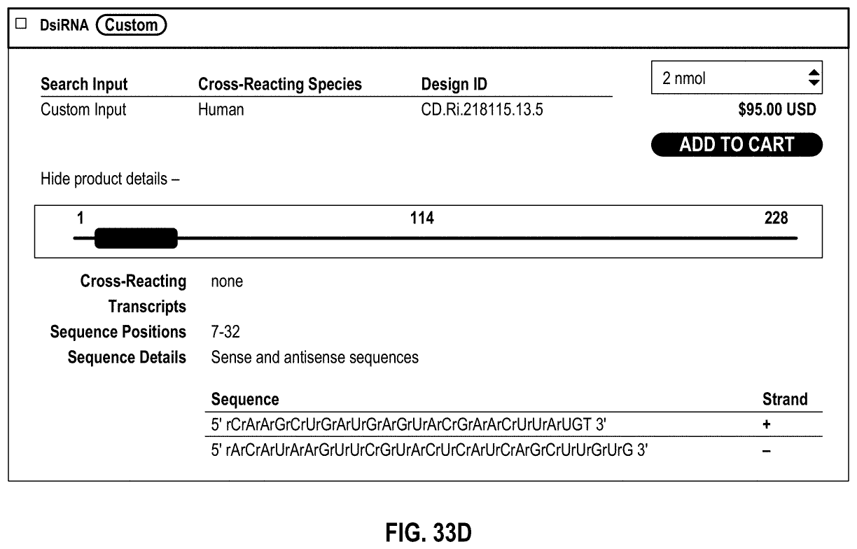

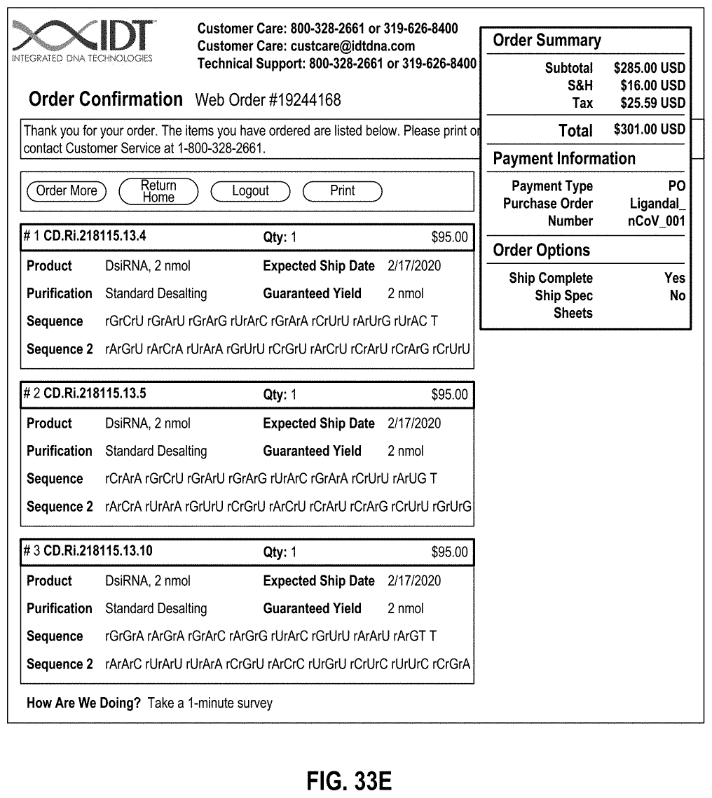

An siRNA was designed for the envelope protein of SARS-CoV-2 using IDT's silencing RNA design tool. The envelope protein is encoded by nts 26,191-26,288 of SEQ ID NO:1. The following sequences were utilized: 13.4 sense (SEQ ID NO:143) and 13.4 antisense (SEQ ID NO:144) (corresponding to nts 26,200-26,224 of SEQ ID NO:1); 13.10 sense (SEQ ID NO:145) and 13.10 antisense (SEQ ID NO:146 (corresponding to nts 26,235-26,259 of SEQ ID NO:1); and 13.5 sense (SEQ ID NO:147) and 13.5 antisense (SEQ ID NO:148) (corresponding to nts 26,207-26,231 of SEQ ID NO:1 FIGS. 33A-33E illustrate the process of designing using the IDT siRNA design tool, including the locations and sequences of selected sense and anti-sense strands.

The CoV-2 scaffold, with or without modification, or with or without immuno-epitope(s), is mixed with the siRNA according to previously developed methods to create a gene vector with a) immune priming activity and vaccine behavior, and b) silencing RNA behavior for the viral replication. See U.S. Provisional Patent Application No. 62/889,496. This approach can also be used for gene editing, RNA editing, and other protein-based Cas tools to treat a variety of viruses.

### Example 13. Computer Simulation of CoV-2 Scaffold Binding to ACE2

This example demonstrates simulation of Scaffold #4, Scaffold #7, Scaffold #8, and Scaffold #9 binding to ACE2.

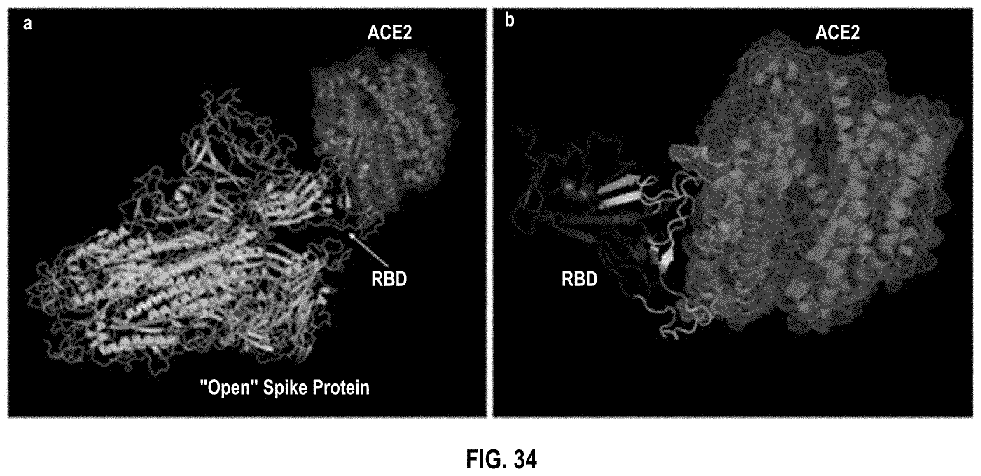

An “align” command was utilized in PyMOL with SARS-CoV-1 bound to ACE2 (PDB ID 6CS2) to approximate the binding interface of the SWISS-MODEL simulated SARS-CoV-2 (left); selected MHC-I and MHC-II epitope regions for inclusion in Scaffold #4 were colored pink and represent P807-K835 and A1020-Y1047 in the 51 spike protein, and were further refined by IEDB immune epitope analysis. FIG. 34. Next, the receptor-binding domain (RBD, blue and multicolored) binding to ACE2 (red) shown on the right in FIG. 34 of the SARS-CoV-2 S1 spike protein was truncated from the larger structure. The resulting RBD structure was run through PDBePISA to determine interacting residues. In the model on the right (FIG. 34), green residues indicate predicted thermodynamically favorable interactions between ACE2 and the S1 spike protein RBD, while yellow indicate predicted thermodynamically neutral and orange indicate predicted thermodynamically unfavorable interactions. Cyan residues indicate the outer bounds of amino acids used to generate SARS-BLOCK™ peptides (V433-V511). While the predicted binding residues did not overlap completely with subsequently empirically-validated sequences, the stretches of amino acids reflected in the simulated motifs accurately reflected binding behavior, whereby N439, Y449, Y453, Q474, G485, N487, Y495, Q498, P499, and Q506 were suggested to be critical ACE2-interfacing residues by the disclosed PDBePISA simulation. Other mutagenesis studies have determined that G446, Y449, Y453, L455, F456, Y473, A475, G476, E484, F486, N487, Y489, F490, Q493, G496, Q498, T500, N501, G502, and Y505 are critical for binding within the stretch of 5425-Y508. (40). Accordingly, residue predictions provided herein can be assessed as being precise, and accurate to within a few amino acids of actual binding behaviors—and represent a rapid and computationally minimalistic way to predict binding protein stretches without a structure when sufficiently long amino acid sequences are employed.

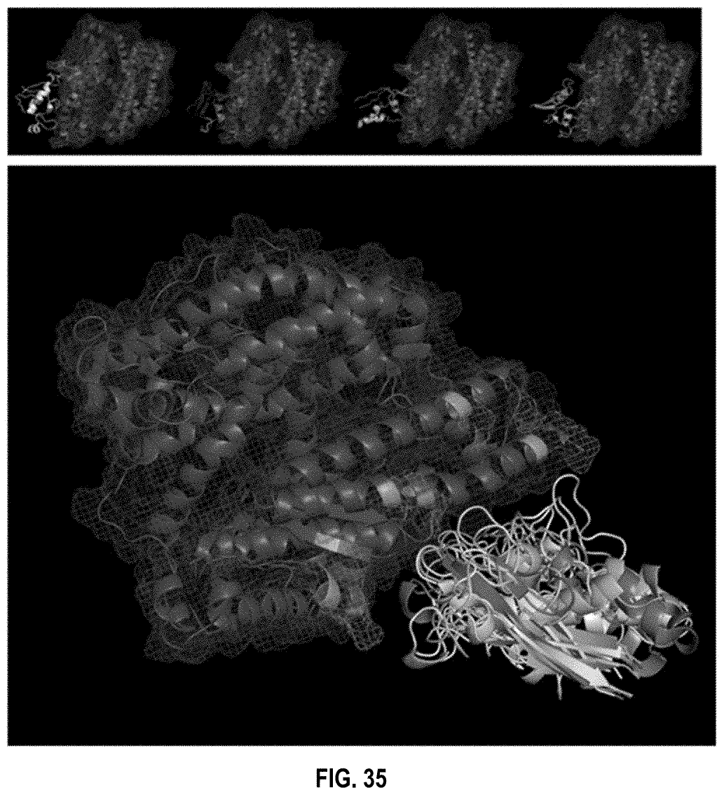

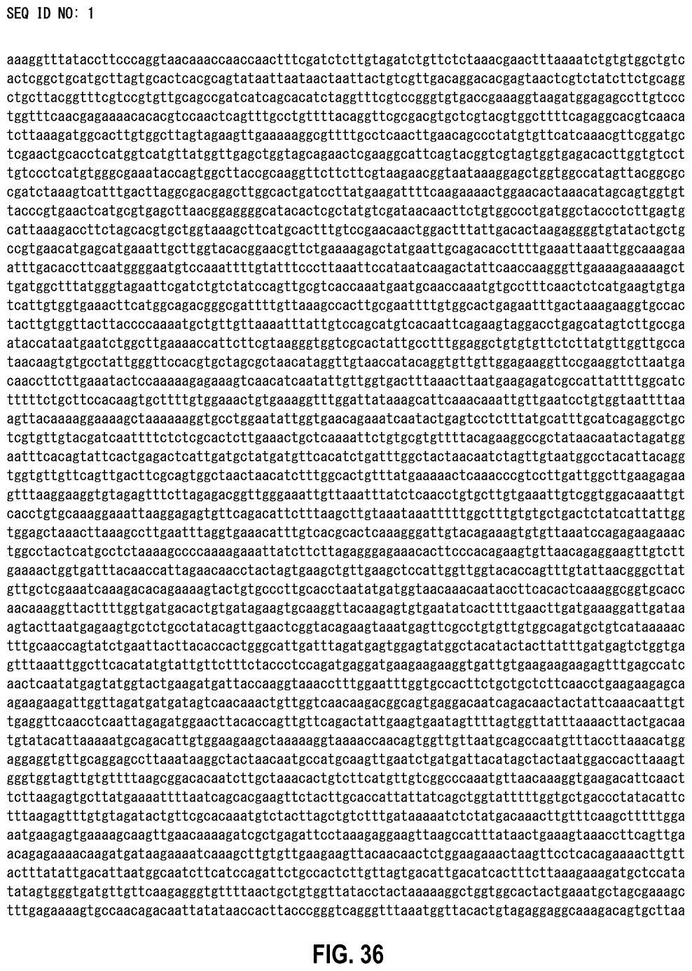

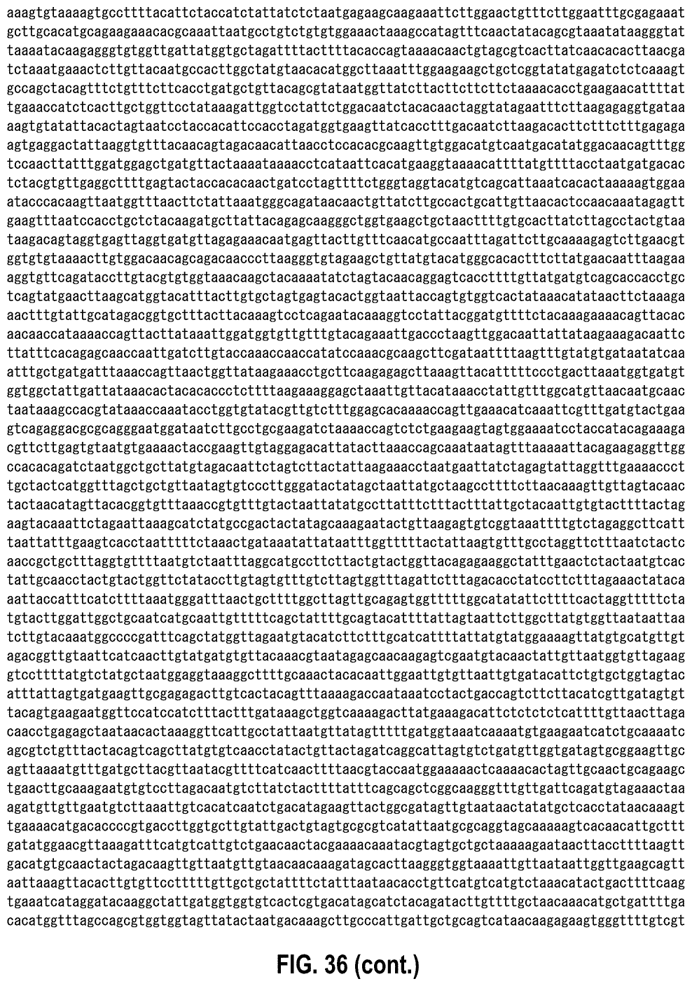

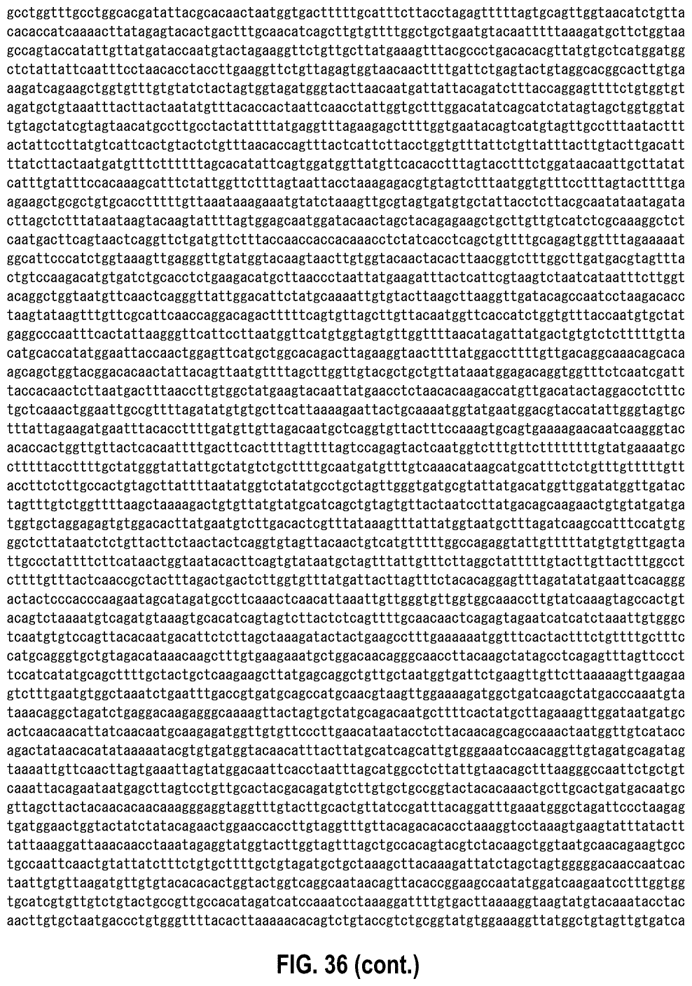

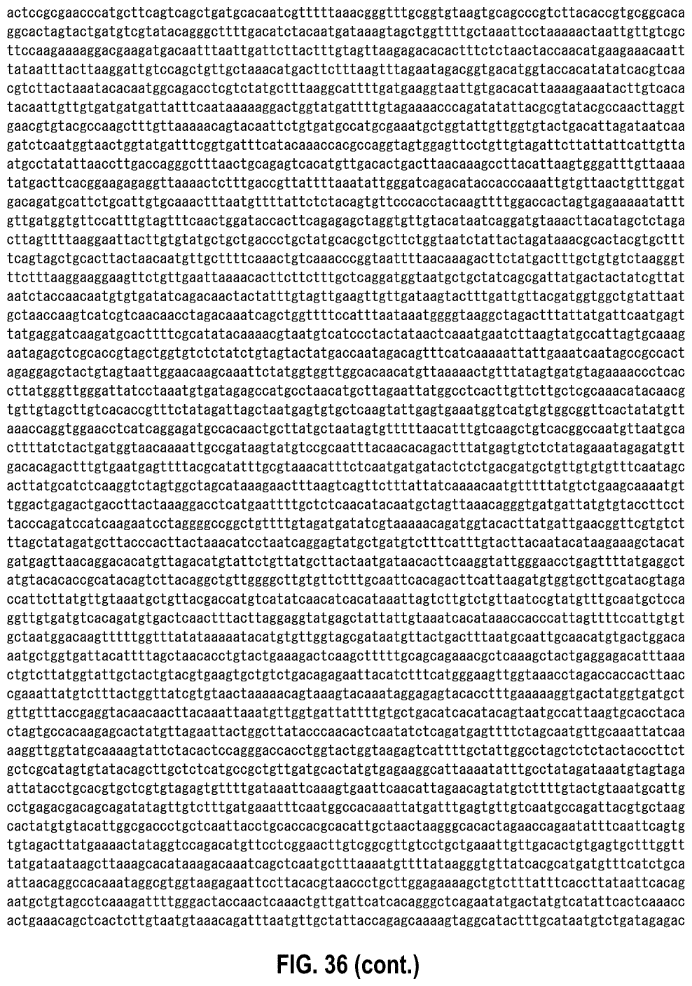

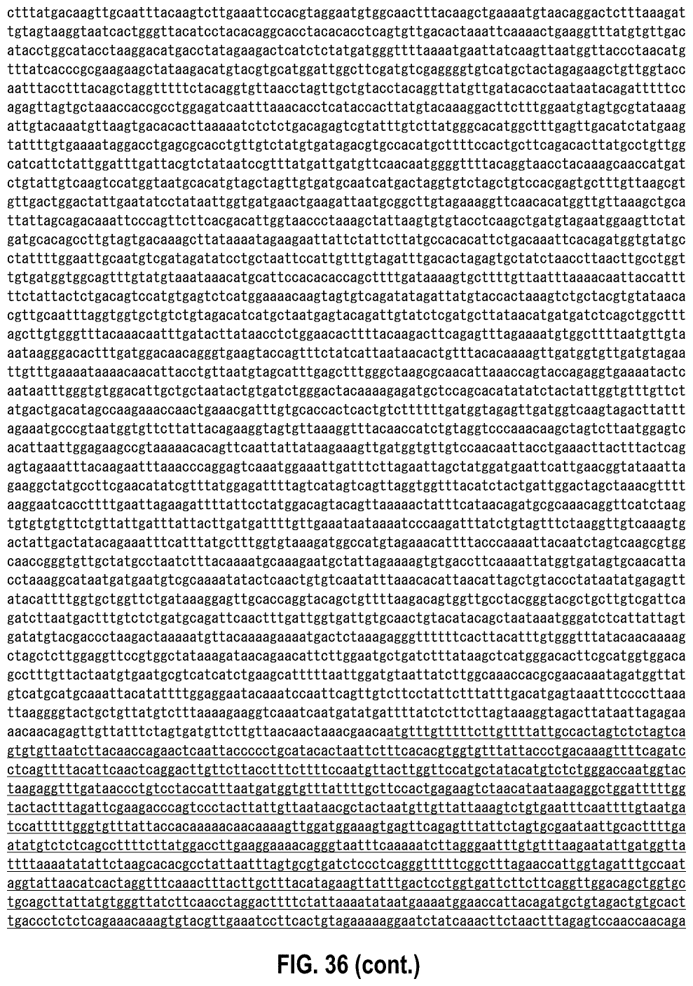

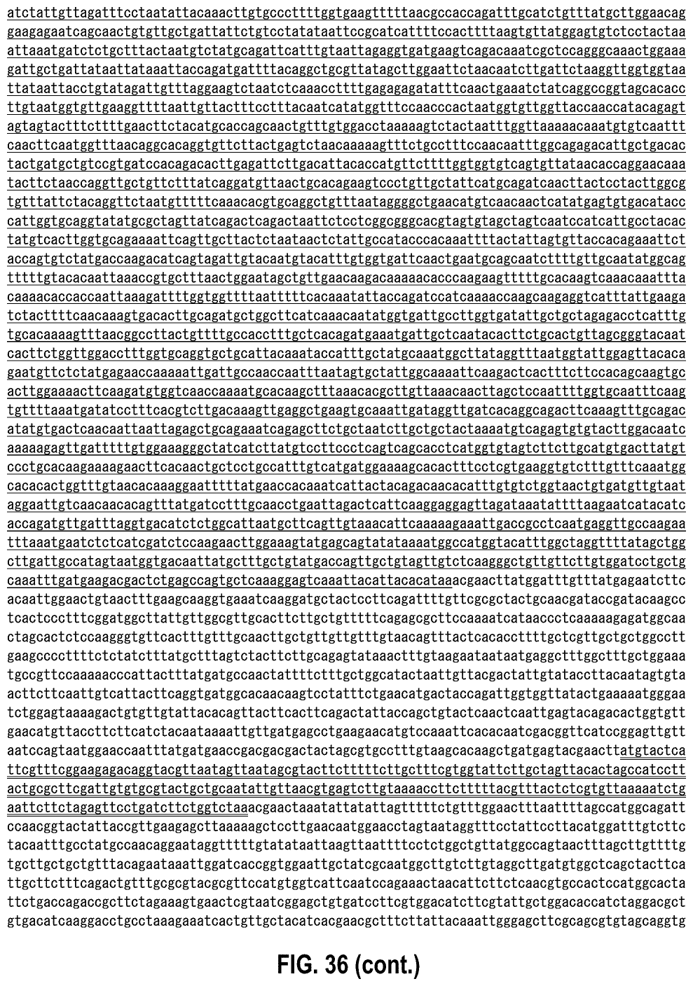

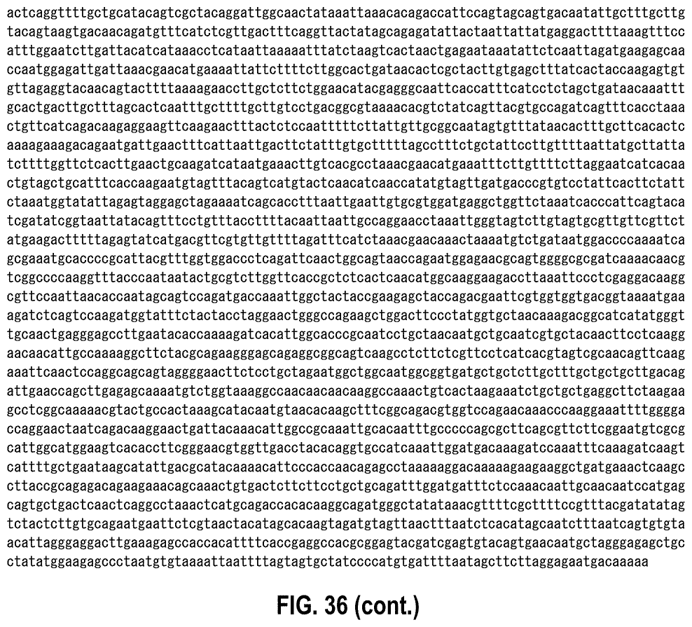

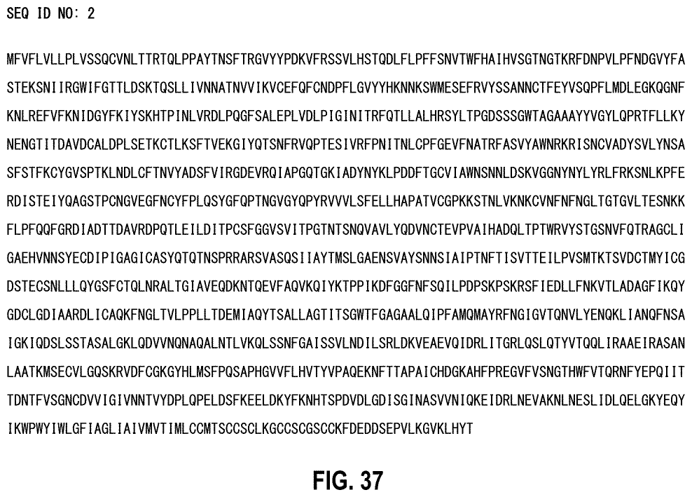

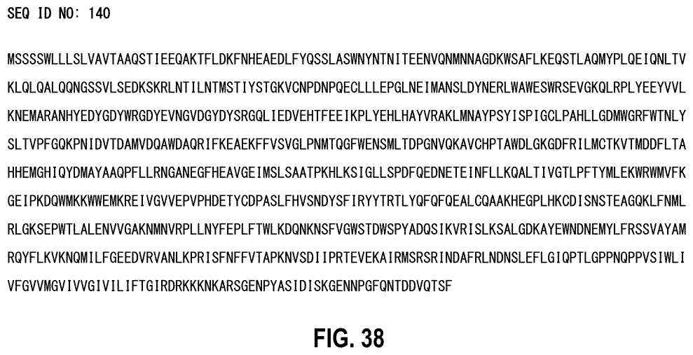

The scaffolds simulated via RaptorX were aligned with the ACE2 receptor (red, with PDBePISA-predicted binding interfaces in green) using the “align” command in PyMOL. See FIG. 35, shown from left to right (top) are Scaffold #4, Scaffold #7, Scaffold #8, and Scaffold #9. Scaffold #4 and Scaffold #7 included the wildtype sequence with two cross-linking motif substitutions. Scaffold #8 included MHC-I and MHC-II epitopes, and Scaffold #9 included a GSGSG linker (white) in one of its non-ACE2-interfacing loop regions. Taking into account all possible folded states generated for each peptide (shown for Scaffold #8 on bottom), these simple align commands can take into account multiple potential conformations of each peptide and may serve as a basis for future studies exploring more advanced molecular dynamics approaches for relaxing and simulating intramolecular interactions at the binding interface. The overlay of many possible folded states represents an electron distribution cloud of possible states that can be simulated for their minimal interfacial free energies with vastly fewer computational resources than are typically required for modeling binding pockets of de novo peptides or protein-protein interfaces that lack existing structures.

From the foregoing, it will be appreciated that specific embodiments of the invention have been described herein for purposes of illustration, but that various modifications may be made without deviating from the scope of the invention. Accordingly, the invention is not limited except as by the appended claims.

### REFERENCES

1. Chi et al. Humanized Single Domain Antibodies Neutralize SARS-CoV-2 by Targeting Spike Receptor Binding Domain. Nat Commun 11(1):4528 (2020)
2. Choy et al. Synthetic peptide studies on the Severe Acute Respiratory Syndrome (SARS) coronavirus spike glycoprotein: Perspective for SARS vaccine development. Clinical Chemistry 50(6):1036-1042 (2004)
3. Bertoni et al. Modeling protein quaternary structure of homo- and hetero-oligomers beyond binary interactions by homology. Scientific Reports 7:10480 (2017)
4. Epelman et al. Detection of soluble angiotensin-converting enzyme 2 in heart failure: insights into the endogenous counter-regulatory pathway of the renin-angiotensin-aldosterone system. J Am Coll Cardiol 52(9):750-754 (2008)
5. Ferretti et al. COVID-19 patients form memory CD8+ T cells that recognize a small set of shared immunodominant epitopes in SARS-CoV-2. Immunity 53(5):P1095-1107 (2020)
6. Hisatake et al. Serum angiotensin-converting enzyme 2 concentration and angiotensin-(1-7) concentration in patients with acute heart failure patients requiring emergency hospitalization. Heart and Vessels 32(3):303-308 (2017)
7. Hoffmann et al. A multibasic cleavage site in the spike protein of SARS-CoV-2 is essential for infection of human lung cells. Mol Cell 78(4):779-784 (2020)
8. Krissinel E and Henrick K. Interference of macromolecular assemblies from crystalline state. J Mol Biol 372(3):774-797 (2007)
9. Mansbach et al. The SARS-CoV-2 Spike Variant D614G Favors an Open Conformational State. bioRxiv (preprint) 2020.07.26.219741 (2020)
10. Nguyen et al. Human leukocyte antigen susceptibility map for Severe Acute Respiratory Syndrome coronavirus 2. J Virol 94(13):e00510-20 (2020)
11. Ogawa et al. The D614G mutation in the SARS-CoV2 Spike protein increases infectivity in an ACE2 receptor dependent manner. bioRxiv (preprint) 2020.07.21.214932 (2020)
12. Poh et al. Two linear epitopes on the SARS-CoV-2 spike protein that elicit neutralizing antibodies in COVID-19 patients. Nature Commun 11:2806 (2020)
13. Remmert et al. HHblits: lightning-fast iterative protein sequence searching by HMM-HMM alignment. Nature Methods 9:173-175 (2012)
14. Soro-Paavonen et al. Circulating ACE2 activity is increased in patients with type 1 diabetes and vascular complications. J Hypertens 30(2):375-383 (2012)
15. Studer et al. QMEANDisCo—distance constraints applied on model quality estimation. Bioinformatics 36(6):1765-1771 (2020)
16. Townend & Tavassoli. Traceless production of cyclic peptide libraries in E. coli. ACS Chemical Biology 11:1624-1630 (2016)
17. Walls et al. Structure, function, and antigenicity of the SARS-CoV-2 spike glycoprotein. Cell 181(2):281-292 (2020)
18. Wang et al. Identification of an HLA-A*0201-restricted CD8+T-cell epitope SSp-1 of SARS-CoV spike protein. Blood 104(1):200-206 (2004)
19. Wang et al. Accurate de novo prediction of protein contact map by ultra-deep learning model. PLoS Computational Biology 13(1):e1005324 (2017)
20. Wang et al. Detection of SARS-CoV-2 in different types of clinical specimens. JAMA 323(18):1843-1844 (2020)
21. Waterhouse et al. SWISS-MODEL: homology modelling of protein structures and complexes. Nucl Acids Res 46:W296-W303 (2018)
22. Wrapp et al. Cryo-EM structure of the 2019-n Co V spike in the prefusion conformation. Science 367:1260-1263 (2020)
23. Zhang et al. The D614G mutation in the SARS-CoV-2 spike protein reduces S1 shedding and increases infectivity. bioRxiv (preprint) 2020.06.12.148726 (2020)
24. Zheng & Song. Novel antibody epitopes dominate the antigenicity of spike glycoprotein in SARS-CoV-2 compared to SARS-CoV. Cell Mol Immunol 17:538-538 (2020)
25. Ou et al. Emergence of RBD mutations in circulating SARS-CoV-2 strains enhancing the structural stability and human ACE2 receptor affinity of the spike protein (2020)
26. Nami et al. The effect of ACE2 inhibitor MLN-4760 on the interaction of SARS-CoV-2 spike protein with human ACE2: a molecular dynamics study (2020)
27. Chour et al. Shared antigen-specific CD8+ T cell Responses against the SARS-CoV-2 spike protein in HLA-A*02:01 COVID-19 participants. medRxiv preprint 2020.05.04.20085779 (2020)
28. Gutierrez et al. Deciphering the TCR repertoire to solve the COVID-19 mystery. Trends in Pharmacological Science 41(8) (2020)
29. Tseng et al. Immunization with SARS coronavirus vaccines leads to pulmonary immunopathology on challenge with the SARS virus. PLoS ONE 7(4):e35421 (2012)
30. Tang et al. Lack of peripheral memory B cell responses in recovered patients with severe acute respiratory syndrome: a six-year follow-up study. The Journal of Immunology 186(12):7264-7268 (2011)
31. Zhang et al. The ORF8 Protein of SARS-CoV-2 Mediates Immune Evasion through Potently Downregulating MHC-I. bioRxiv preprint 2020.05.24.111823 (2020)
32. Diao et al. Reduction and functional exhaustion of T cells in patients with coronavirus disease 2019 (COVID-19). Frontiers in Immunology 11(827) (2020)
33. Zheng et al. Elevated exhaustion levels and reduced functional diversity of T cells in peripheral blood may predict severe progression in COVID-19 patients. Cellular & molecular immunology 17(5):541-543 (2020)
34. Kumar et al. An in-silico based clinical insight on the effect of noticeable CD4 conserved residues of SARS-CoV-2 on the CD4-MHC-11 interactions. bioRxiv preprint 2020.06.19.161802 (2020)
35. Woodruff et al. Critically ill SARS-CoV-2 patients display lupus-like hallmarks of extrafollicular B cell activation. medRxiv preprint 2020.04.29.20083717 (2020)
36. Woodruff et al. Extra follicular B cell responses correlate with neutralizing antibodies and morbidity in COVID-19. Nature Immunol 21:1506-1516 (2020)
37. Kellam & Barclay. The dynamics of humoral immune responses following SARS-Co V-2 infection and the potential for reinfection. Journal of General Virology 101:791-797 (2020)
38. Seow et al. Longitudinal evaluation and decline of antibody responses in SARS-CoV-2 infection. medRxiv preprint 2020.07.09.20148429 (2020)
39. Robbiani et al. Convergent antibody responses to SARS-CoV-2 infection in convalescent individuals. Nature 584(7821):437-442 (2020)
40. Yi et al. Key residues of the receptor binding motif in the spike protein of SARS-CoV-2 that interact with ACE2 and neutralizing antibodies. Cellular & Molecular Immunology 17:621-630 (2020)

TABLE 1
Representative SARS-CoV-2 scaffolds
Representative examples of scaffolds derived from SARS-CoV-2 S protein
are set forth in Table 1 below. Critical binding motifs are underlined,
immune-epitopes are bolded and italicized, linkers are bolded, substi-
tutions are double underlined, poly charged N- and C-terminus amino
acid residues are squiggly underlined, and EPEA C-term tags are
italicized.

| | | SEQ | |
|---|---|---|---|
| | | ID | |
| Name | Sequence | NO | Modifications |
| Scaf- | VIAW [symbol] SKVGGNYNYLYRLFRKSN [symbol] | 72 | Corresponds to residues 433-511 |
| fold | [symbol] STEIYQAGSTPCNGVEGFNCYF | | of wildtype SARS-CoV-2 S |
| #1 | PLQSY [symbol] NGVG [symbol] RVV | | protein. |
| Scaf- | VIAW [symbol] SKVGGNYNYLYRL [symbol] | 73 | Corresponds to residues 433-511 |
| fold | [symbol] QA [symbol] GFNCYFPLQS | | of CoV-2 S protein, but with |
| #2 | Y [symbol] NGVG [symbol] RVV | | backbone region between two |
| | | | critical binding motifs and |
| | | | partial sequence of the second |
| | | | critical binding motif replaced |
| | | | with two immuno-epitopes. |
| Scaf- | VIAW [symbol] SKVGGNYNYLYRLGSGSG | 74 | Corresponds to residues 433-511 |
| fold | QAGSTPCNGVEGFNCYFPLQSY [symbol] N | | of CoV-2 S protein, but with |
| #3 | GVG [symbol] RVV | | backbone region between two |
| | | | critical binding motifs replaced |
| | | | with GS linker and repulsory |
| | | | residue Y473 deleted. |
| | | | Identical to Scaffold #41 but for |
| | | | absence of I434K substitution. |
| | | | Identical to Scaffold #42 but for |
| | | | absence of A435K substitution. |
| Scaf- | VIAW [symbol] SKVGGNYNYLYRCFRKSN [symbol] | 75 | Corresponds to residues 433-511 |
| fold | [symbol] STEIYQAGSTPGNGVEGFNGYF | | of CoV-2 S protein, but with |
| #4 | CLQSY [symbol] NGVG [symbol] RVV | | L455C and P491C substitutions to |
| | | | introduce a disulfide bond and |
| | | | C480G and C488G substitutions. |
| | | | Identical to Scaffold #7 but for |
| | | | absence of two poly charged amino |
| | | | acid residues added to the N- |
| | | | terminus and three poly charged |
| | | | amino acid residues added to the |
| | | | C-terminus. |
| Scaf- | VIAW [symbol] SKVGGNYNYLYRC [symbol] | 76 | Corresponds to residues 433-511 |
| fold | [symbol] QA [symbol] GFNGYFCLQ | | of CoV-2 S protein, but with |
| #5 | SY [symbol] NGVG [symbol] RVV | | backbone region between two |
| | | | critical binding motifs and |
| | | | partial sequence of the second |
| | | | critical binding motif replaced |
| | | | with two immuno-epitopes, L455C |
| | | | and P491C substitutions to |
| | | | introduce a disulfide bond, and |
| | | | C488G substitution. |
| Scaf- | VIAW [symbol] SKVGGNYNYLYRCGSGSG | 77 | Corresponds to residues 433-511 |
| fold | QAGSTPGNGVEGFNGYFCLQSYN | | of CoV-2 S protein, but with |
| #6 | GVG [symbol] RVV | | backbone region between two |
| | | | critical binding motifs replaced |
| | | | with GS linker, repulsory residue |
| | | | Y473 deleted, L455C and P491C |
| | | | substitutions to introduce a |
| | | | disulfide bond, and C480G and |
| | | | C488G substitutions. |
| | | | Identical to Scaffold #9 but for |
| | | | absence of two poly charged amino |
| | | | acid residues added to the N- |
| | | | terminus and three poly charged |
| | | | amino acid residues added to the |
| | | | C-terminus. |
| Scaf- | [symbol] VIAW [symbol] SKVGGNYNYLYRCFRK | 78 | Corresponds to residues 433-511 |
| fold | SN [symbol] STEIYQAGSTPGNGVEGFN | | of CoV-2 S protein, but with |
| #7 | GYFCLQSY [symbol] NGVG [symbol] RVV [symbol] | | L455C and P491C substitutions |
| | | | to introduce a disulfide bond, |
| | | | C480G and C488G substitutions, |
| | | | and two poly charged amino acid |
| | | | residues added to the N-terminus |
| | | | and three poly charged amino acid |
| | | | residues added to the C-terminus. |
| | | | Identical to Scaffold #4 but for |
| | | | addition of two poly charged amino |
| | | | acid residues added to the N- |
| | | | terminus and three poly charged |
| | | | amino acid residues added to the |
| | | | C-terminus. |
| Scaf- | [symbol] VIAW [symbol] SKVGGNYNYLYRC [symbol] | 79 | Corresponds to residues 433-511 |
| fold | [symbol] QA [symbol] GFNGYFC | | of CoV-2 S protein, but with |
| #8 | LQSY [symbol] NGVG [symbol] RVV [symbol] | | backbone region between two |
| | | | critical binding motifs and |
| | | | partial sequence of the second |
| | | | critical binding motif replaced |
| | | | with two immuno-epitopes, L455C |
| | | | and P491C substitutions to in- |
| | | | troduce a disulfide bond, C488G |
| | | | substitution, and two poly |
| | | | charged amino acid residues added |
| | | | to the N-terminus and three poly |
| | | | charged amino acid residues added |
| | | | to the C-terminus. |
| | | | Identical to Scaffold #40 but for |
| | | | absence of C to S substitution in |
| | | | the first inserted immune- |
| | | | epitope. |
| Scaf- | [symbol] VIAW [symbol] SKVGGNYNYLYRCGSG | 80 | Corresponds to residues 433-511 |
| fold | SGQAGSTPGNGVEGFNGYFCLQSY [symbol] | | of CoV-2 S protein, but with |
| #9 | [symbol] NGVG [symbol] RVV [symbol] | | backbone region between two |
| | | | critical binding motifs replaced |
| | | | with GS linker, repulsory residue |
| | | | Y473 deleted, L455C and P491C |
| | | | substitutions to introduce a di- |
| | | | sulfide bond, C480G and C488G |
| | | | substitutions, and two poly |
| | | | charged amino acid residues added |
| | | | to the N-terminus and three poly |
| | | | charged amino acid residues added |
| | | | to the C-terminus. |
| | | | Identical to Scaffold #6 but for |
| | | | addition of two poly charged |
| | | | amino acid residues added to the |
| | | | N-terminus and three poly charged |
| | | | amino acid residues added to the |
| | | | C-terminus. |
| Scaf- | [symbol] SKVGGNYNYLYRLFRKSN [symbol] | 81 | Corresponds to residues 437-508 |
| fold | [symbol] STEIYQAGSTPCNGVEGFNCYFPLQS | | of wildtype SARS-CoV-2 S protein. |
| #10 | Y [symbol] NGVG [symbol] | | |
| Scaf- | [symbol] SKVGGNYNYLYRLGSGSGSQAG | 82 | Corresponds to residues 437-508 |
| fold | STPCNGVEGFNCYFPLQSY [symbol] NGVG | | of SARS-CoV-2 S protein, but |
| #11 | [symbol] | | with backbone region between |
| | | | two critical binding motifs |
| | | | replaced with GS linker and |
| | | | repulsory residue Y473 deleted. |
| Scaf- | [symbol] SKVGGNYNYLYRLGSGSGQAGS | 83 | Corresponds to residues 437-508 |
| fold | TPCNGVEGFNCYFPLQSY [symbol] NGVG [symbol] | | of SARS-CoV-2 S protein, but |
| #12 | [symbol] | | with backbone region between |
| | | | two critical binding motifs |
| | | | replaced with GS linker and |
| | | | repulsory residue Y473 deleted. |
| Scaf- | [symbol] SKVGGNYNYLYRLGSGSQAGST | 84 | Corresponds to residues 437-508 |
| fold | PCNGVEGFNCYFPLQSY [symbol] NGVG [symbol] | | of SARS-CoV-2 S protein, but |
| #13 | [symbol] | | with backbone region between |
| | | | two critical binding motifs |
| | | | replaced with GS linker and |
| | | | repulsory residue Y473 deleted. |
| Scaf- | [symbol] SKVGGNYNYLYRLGSGQAGSTP | 85 | Corresponds to residues 437-508 |
| fold | CNGVEGFNCYFPLQSY [symbol] NGVG [symbol] | | of SARS-CoV-2 S protein, but |
| #14 | [symbol] | | with backbone region between |
| | | | two critical binding motifs |
| | | | replaced with GS linker and |
| | | | repulsory residue Y473 deleted. |
| Scaf- | [symbol] SKVGGNYNYLYRL [symbol] | 86 | Corresponds to residues 437-508 |
| fold | [symbol] QA [symbol] GFNCYFPLQSY [symbol] | | of SARS-CoV-2 S protein, but |
| #15 | [symbol] NGVG [symbol] | | with backbone region between |
| | | | two critical binding motifs |
| | | | and partial sequence of the |
| | | | second critical binding motif |
| | | | replaced with two immuno- |
| | | | epitopes. |
| | | | Identical to Scaffold #2 but |
| | | | for absence of N-terminal VIAW |
| | | | and C-terminal RVV. |
| Scaf- | [symbol] SKVGGNYNYLYRLFR [symbol] | 87 | Corresponds to residues 437-508 |
| fold | [symbol] QA [symbol] GFNCYFPLQSY | | of SARS-CoV-2 S protein, but |
| #16 | [symbol] NGVG [symbol] | | with majority of backbone region |
| | | | between two critical binding |
| | | | motifs and partial sequence of |
| | | | the second critical binding motif |
| | | | replaced with two immuno- |
| | | | epitopes. |
| Scaf- | [symbol] SKVGGNYNYLYRLFRKS [symbol] | 88 | Corresponds to residues 437-508 |
| fold | [symbol] QA [symbol] GFNCYFPLQS | | of SARS-CoV-2 S protein, but |
| #17 | Y [symbol] NGVG [symbol] | | with majority of backbone region |
| | | | between two critical binding |
| | | | motifs and partial sequence of |
| | | | the second critical binding motif |
| | | | replaced with two immuno- |
| | | | epitopes. |
| Scaf- | [symbol] SKVGGNYNYLYRLFRKSN [symbol] | 89 | Corresponds to residues 437-508 |
| fold | [symbol] QA [symbol] GFNCYFPL | | of SARS-CoV-2 S protein, but |
| #18 | QSY [symbol] NGVG [symbol] | | with majority of backbone region |
| | | | between two critical binding |
| | | | motifs and partial sequence of |
| | | | the second critical binding motif |
| | | | replaced with two immuno- |
| | | | epitopes. |
| Scaf- | [symbol] SKVGGNYNYLYRLFRKSNL [symbol] | 90 | Corresponds to residues 437-508 |
| fold | [symbol] QA [symbol] GFNCYFPL | | of SARS-CoV-2 S protein, but |
| #19 | QSY [symbol] NGVG [symbol] | | with majority of backbone region |
| | | | between two critical binding |
| | | | motifs and partial sequence of |
| | | | the second critical binding motif |
| | | | replaced with two immuno- |
| | | | epitopes. |
| Scaf- | [symbol] SKVGGNYNYLYRLFRKSNLK [symbol] | 91 | Corresponds to residues 437-508 |
| fold | [symbol] QA [symbol] GFNCYF | | of SARS-CoV-2 S protein, but |
| #20 | PLQSY [symbol] NGVG [symbol] | | with majority of backbone region |
| | | | between two critical binding |
| | | | motifs and partial sequence of |
| | | | the second critical binding motif |
| | | | replaced with two immuno- |
| | | | epitopes. |
| Scaf | VIAW [symbol] SKVGGNYNYKYRLFRKSN [symbol] | 92 | Corresponds to residues 433-511 |
| fold- | [symbol] SNEIYQAGSTPCNGVPGFNCYF | | of SARS-CoV-2 S protein, but |
| #21 | PLQSY [symbol] TGVG [symbol] RVV | | with N439R, L452K, T470N, E484P, |
| | | | and N501T substitutions to |
| | | | increase affinity for ACE2 and |
| | | | antibodies. |
| Scaf- | [symbol] VIAW [symbol] SKVGGNYNYLYRC [symbol] | 93 | Corresponds to residues 433-511 |
| fold | [symbol] QA [symbol] QA [symbol] | | of SARS-CoV-2 S protein, but |
| #22 | [symbol] [symbol] RVV [symbol] | | with backbone region between |
| | | | two critical binding motifs and |
| | | | partial sequence of the second |
| | | | critical binding motif replaced |
| | | | with two immuno-epitopes, L455C |
| | | | and P491C substitutions to in- |
| | | | troduce a disulfide bond, C488G |
| | | | substitution, and two poly |
| | | | charged amino acid residues |
| | | | added to the N-terminus and |
| | | | three poly charged amino acid |
| | | | residues added to the C-terminus. |
| | | | Identical to Scaffold #8 but for |
| | | | addition of QA between second |
| | | | inserted immune-epitope and |
| | | | second critical binding motif. |
| | | | Identical to Scaffold #24 but |
| | | | for absence of EPEA C-term tag. |
| Scaf- | [symbol] VIAW [symbol] SKVGGNYNYLYRCFRK | 94 | Corresponds to residues 433-511 |
| fold | SN [symbol] STEIYQAGSTPCNGVEGFN | | of CoV-2 S protein, but with |
| #23 | GYFCLQSY [symbol] NGVG [symbol] RVV [symbol] | | L455C and P491C substitutions |
| | | | to introduce a disulfide bond, |
| | | | C488G substitution, and two poly |
| | | | charged amino acid residues added |
| | | | to the N-terminus and three poly |
| | | | charged amino acid residues added |
| | | | to the C-terminus. |
| | | | Identical to Scaffold #7 but for |
| | | | absence of C480G substitution. |
| Scaf- | [symbol] VIAW [symbol] SKVGGNYNYLYRC [symbol] | 95 | Corresponds to residues 433-511 |
| fold | [symbol] QA [symbol] QA [symbol] | | of SARS-CoV-2 S protein, but |
| #24 | [symbol] RVV [symbol] EPE | | with backbone region between |
| | A | | two critical binding motifs and |
| | | | partial sequence of the second |
| | | | critical binding motif replaced |
| | | | with two immuno-epitopes, L455C |
| | | | and P491C substitutions to in- |
| | | | troduce a disulfide bond, C488G |
| | | | substitution, two poly charged |
| | | | amino acid residues added to the |
| | | | N-terminus and three poly charged |
| | | | amino acid residues added to the |
| | | | C-terminus, and EPEA C-tag. |
| | | | Identical to Scaffold #22 but for |
| | | | addition of EPEA C-term tag. |
| Scaf- | [symbol] VIAW [symbol] SKVGGNYNYLYRC [symbol] | 96 | Corresponds to residues 433-511 |
| fold | [symbol] QA [symbol] QA [symbol] | | of SARS-CoV-2 S protein, but |
| #25 | [symbol] RVV [symbol] EPE | | with backbone region between |
| | A | | two critical binding motifs and |
| | | | partial sequence of the second |
| | | | critical binding motif replaced |
| | | | with two immuno-epitopes, C to |
| | | | S substitution in the first in- |
| | | | serted immune-epitope, L455C |
| | | | and P491C substitutions to |
| | | | introduce a disulfide bond, |
| | | | C488G substitution, two poly |
| | | | charged amino acid residues |
| | | | added to the N-terminus and |
| | | | three poly charged amino acid |
| | | | residues added to the C- |
| | | | terminus, and EPEA C-tag. |
| Scaf- | [symbol] VIAW [symbol] SKVGGNYNYLYRL [symbol] | 97 | Free self-folded peptide. |
| fold | [symbol] QA [symbol] QA [symbol] | | Corresponds to residues 433-511 |
| #26 | [symbol] RVV [symbol] EPE | | of SARS-CoV-2 S protein, but |
| | A | | with backbone region between |
| | | | two critical binding motifs and |
| | | | partial sequence of the second |
| | | | critical binding motif replaced |
| | | | with two immuno-epitopes, P491C |
| | | | substitution, C488G substitution, |
| | | | two poly charged amino acid |
| | | | residues added to the N-terminus |
| | | | and three poly charged amino acid |
| | | | residues added to the C-terminus, |
| | | | and EPEA C-tag. |
| Scaf- | EVEVEFEVEVIAW [symbol] SKVGGNYNYL | 98 | Free self-folded peptide. |
| fold | YRLFGSGSGSGSGSGSGSGSYQAGSTPC | | Corresponds to residues 433-511 |
| #27 | NGVEGFNSYFPLQSY [symbol] NGVG [symbol] | | of SARS-CoV-2 S protein, but |
| | RVVRVRFRVRVREPEA | | with majority of backbone region |
| | | | between two critical binding |
| | | | motifs replaced with GS linker, |
| | | | C488S substitution, filler |
| | | | residues added to N- and C- |
| | | | termini, and EPEA C-tag. |
| | | | Identical to Scaffold #28 but |
| | | | for absence of L455C and P491C |
| | | | substitutions. |
| Scaf- | EVEVEFEVEVIAW [symbol] SKVGGNYNYL | 99 | Free disulfide bonded peptide. |
| fold | YRCFGSGSGSGSGSGSGSGSYQAGSTP | | Corresponds to residues 433-511 |
| #28 | CNGVEGFNSYFCLQSY [symbol] NGVG [symbol] | | of SARS-CoV-2 S protein, but |
| | [symbol] RVVRVRFRVRVREPEA | | with majority of backbone region |
| | | | between two critical binding |
| | | | motifs replaced with GS linker, |
| | | | L455C and P491C substitutions to |
| | | | introduce a disulfide bond, |
| | | | C488S substitution, filler |
| | | | residues added to N- and C- |
| | | | termini, and EPEA C-tag. |
| | | | Identical to Scaffold #27 but |
| | | | for addition of L455C and P491C |
| | | | substitutions. |
| | | | Identical to Scaffold #30, but |
| | | | with GS linker in place of |
| | | | three inserted TCR epitopes. |
| Scaf- | EVEVEFEVEVIAW [symbol] SKVGGNYNYL | 100 | Free self-folded peptide. |
| fold | YRLFKLWAQCVQLYLQPRTFLLLLYDANY | | Corresponds to residues 433-511 |
| #29 | FLYQAGSTPCNGVEGFNSYFPLQSY [symbol] | | of SARS-CoV-2 S protein, but |
| | [symbol] NGVG [symbol] RVVRVRFRVRVREPEA | | with majority of backbone region |
| | | | between two critical binding |
| | | | motifs replaced with three TCR |
| | | | epitopes (KLWAQCVQL, LLYDANYFL, |
| | | | and YLQPRTFLL), C488S substi- |
| | | | tution, filler residues added |
| | | | to N- and C-termini, and EPEA |
| | | | C-tag. |
| | | | Identical to Scaffold #30 but |
| | | | for absence of L455C and P491C |
| | | | substitutions. |
| Scaf- | EVEVEFEVEVIAW [symbol] SKVGGNYNYL | 101 | Free disulfide bonded peptide. |
| fold | YRCFKLWAQCVQLYLQPRTFLLLLYDANY | | Corresponds to residues 433-511 |
| #30 | FLYQAGSTPCNGVEGFNSYFCLQSY [symbol] | | of SARS-CoV-2 S protein, but |
| | [symbol] NGVG [symbol] RVVRVRFRVRVREPEA | | with majority of backbone region |
| | | | between two critical binding |
| | | | motifs replaced with three TCR |
| | | | epitopes (KLWAQCVQL, LLYDANYFL, |
| | | | and YLQPRTFLL), L455C and P491C |
| | | | substitutions to introduce a |
| | | | disulfide bond, C488S substitu- |
| | | | tion, filler residues added to |
| | | | N- and C-termini, and EPEA C-tag. |
| | | | Identical to Scaffold #28, but |
| | | | with three TCR epitopes in place |
| | | | of inserted GS linker. |
| | | | Identical to Scaffold #29 but |
| | | | for addition of L455C and P491C |
| | | | substitutions. |
| Scaf- | CEVEVEFEVEVIAW [symbol] SKVGGNYN | 102 | Conjugatable peptide. |
| fold | YKYRLFKLWAQCVQLYLQPRTFLLLLYDA | | Corresponds to residues 433-511 |
| #31 | NYFLYQAGSTPCNGVEGFNSYFPLQSY [symbol] | | of SARS-CoV-2 S protein, but |
| | [symbol] TGVG [symbol] RRREPEA | | with majority of backbone region |
| | | | between two critical binding |
| | | | motifs replaced with three TCR |
| | | | epitopes (KLWAQCVQL, LLYDANYFL, |
| | | | and YLQPRTFLL), N439R, L452K, |
| | | | C488S, and N501T substitutions, |
| | | | filler residues added to N- and |
| | | | C-termini, and EPEA C-tag. |
| | | | Identical to Scaffold #32, but |
| | | | with three TCR epitopes in place |
| | | | of inserted GS linker. |
| Scaf- | CEVEVEFEVEVIAW [symbol] SKVGGNYN | 103 | Conjugatable peptide. |
| fold | YKYRLFGSGSGSGSGSGSGSGSYQAGST | | Corresponds to residues 433-511 |
| #32 | PCNGVEGFNSYFPLQSY [symbol] TGVG [symbol] | | of SARS-CoV-2 S protein, but |
| | [symbol] RRREPEA | | with majority of backbone region |
| | | | between two critical binding |
| | | | motifs replaced with GS linker, |
| | | | N439R, L452K, C488S, and N501T |
| | | | substitutions, filler residues |
| | | | added to N- and C-termini, |
| | | | and EPEA C-tag. |
| | | | Identical to Scaffold #31, but |
| | | | with GS linker in place of three |
| | | | inserted TCR epitopes. |
| Scaf- | CEVEVEFEVEVIAW [symbol] SKVGGNYN | 104 | Conjugatable peptide. |
| fold | YKYRLFKLWAQCVQLYLQPRTFLLLLYDA | | Corresponds to residues 433-511 |
| #33 | NYFLNEIYQAGSTPCNGVEGFNSYFPLQS | | of SARS-CoV-2 S protein, but |
| | Y [symbol] TGVG [symbol] RRREPEA | | with majority of backbone region |
| | | | between two critical binding |
| | | | motifs replaced with three TCR |
| | | | epitopes (KLWAQCVQL, LLYDANYFL, |
| | | | and YLQPRTFLL) and N residue, |
| | | | N439R, L452K, C488S, and N501T |
| | | | substitutions, filler residues |
| | | | added to N- and C-termini, and |
| | | | EPEA C-tag. |
| | | | Identical to Scaffold #34, but |
| | | | with three TCR epitopes in place |
| | | | of inserted GS linker. |
| Scaf- | CEVEVEFEVEVIAW [symbol] SKVGGNYN | 105 | Conjugatable peptide. |
| fold | YKYRLFGSGSGSGSGSGSGSGSNEIYQA | | Corresponds to residues 433-511 |
| #34 | GSTPCNGVEGFNSYFPLQSY [symbol] TGV | | of SARS-CoV-2 S protein, but |
| | G [symbol] RRREPEA | | with majority of backbone region |
| | | | between two critical binding |
| | | | motifs replaced with GS linker |
| | | | and N residue, N439R, L452K, |
| | | | C488S, and N501T substitutions, |
| | | | filler residues added to N- and |
| | | | C-termini, and EPEA C-tag. |
| | | | Identical to Scaffold #33, but |
| | | | with GS linker in place of |
| | | | three inserted TCR epitopes. |
| Scaf- | EVEVEFEVEVIAW [symbol] SKVGGNYNYL | 106 | Free self-folded peptide. |
| fold | YRLF [symbol] QA [symbol] | | Corresponds to residues 433-511 |
| #35 | Q [symbol] [symbol] | | of SARS-CoV-2 S protein, but |
| | [symbol] RVVRVRFRVRVREPEA | | with majority of backbone region |
| | | | between two critical binding |
| | | | motifs replaced with two immune- |
| | | | epitopes, C to S substitution in |
| | | | the first inserted immune- |
| | | | epitope, C488S substitution, |
| | | | filler residues added to N- and |
| | | | C-termini, and EPEA C-tag. |
| | | | Identical to Scaffold #36 but |
| | | | for absence of L455C and P491C |
| | | | substitutions. |
| Scaf- | EVEVEFEVEVIAW [symbol] SKVGGNYNYL | 107 | Free disulfide bonded peptide. |
| fold | YRCF [symbol] QA [symbol] | | Corresponds to residues 433-511 |
| #36 | Q [symbol] [symbol] | | of SARS-CoV-2 S protein, but |
| | [symbol] RVVRVRFRVRVREPEA | | with majority of backbone region |
| | | | between two critical binding |
| | | | motifs replaced with two immune- |
| | | | epitopes, C to S substitution in |
| | | | the first inserted immune- |
| | | | epitope, L455C and P491C substi- |
| | | | tutions to introduce a disulfide |
| | | | bond, C488S substitution, filler |
| | | | residues added to N- and C- |
| | | | termini, and EPEA C-tag. |
| | | | Identical to Scaffold #35 but |
| | | | for addition of L455C and P491C |
| | | | substitutions. |
| Scaf- | CEVEVEFEVEVIAW [symbol] SKVGGNYN | 108 | Conjugatable peptide. |
| fold | YKYRLF [symbol] QA [symbol] | | Corresponds to residues 433-511 |
| #37 | [symbol] Q [symbol] [symbol] | | of SARS-CoV-2 S protein, but |
| | P [symbol] RRREPEA | | with majority of backbone region |
| | | | between two critical binding |
| | | | motifs replaced with two immune- |
| | | | epitopes, C to S substitution in |
| | | | the first inserted immune- |
| | | | epitope, N439R, L452K, C488S, |
| | | | and N501T substitutions, filler |
| | | | residues added to N- and C- |
| | | | termini, and EPEA C-tag. |
| Scaf- | CEVEVEFEVEVIAW [symbol] SKVGGNYN | 109 | Conjugatable peptide. |
| fold | YKYRLF [symbol] QA [symbol] | | Corresponds to residues 433-511 |
| #38 | [symbol] QNEI [symbol] [symbol] | | of SARS-CoV-2 S protein, but |
| | [symbol] RRREPEA | | with majority of backbone region |
| | | | between two critical binding |
| | | | motifs replaced with two immune- |
| | | | epitopes, C to S substitution in |
| | | | the first inserted immune- |
| | | | epitope, N439R, L452K, C488S, |
| | | | and N501T substitutions, filler |
| | | | residuesadded to N- and C- |
| | | | termini, and EPEA C-tag. |
| Scaf- | [symbol] VIAW [symbol] SKVGGNYNYLYRCGSG | 110 | Corresponds to residues 433-511 |
| fold | SGQAGSTPCNGVEGFNGYFCLQSY [symbol] | | of SARS-CoV-2 S protein, but |
| #39 | [symbol] NGVG [symbol] RVV [symbol] EPEA | | with backbone region between |
| | | | two critical binding motifs |
| | | | replaced with GS linker, |
| | | | repulsory residue Y473 deleted, |
| | | | L455C and P491C substitutions |
| | | | to introduce a disulfide bond, |
| | | | C488G substitution, two poly |
| | | | charged amino acid residues |
| | | | added to the N-terminus and |
| | | | three poly charged amino |
| | | | acid residues added to the |
| | | | C-terminus, and EPEA C-tag. |
| Scaf- | [symbol] VIAW [symbol] SKVGGNYNYLYRC [symbol] | 111 | Corresponds to residues 433-511 |
| fold | [symbol] QA [symbol] [symbol] | | of SARS-CoV-2 S protein, but |
| #40 | [symbol] RVV [symbol] | | with backbone region between |
| | | | two critical binding motifs |
| | | | and partial sequence of the |
| | | | second critical binding motif |
| | | | replaced with two immuno- |
| | | | epitopes, C to S substitution |
| | | | in the first inserted immune- |
| | | | epitope, L455C and P491C |
| | | | substitutions to introduce a |
| | | | disulfide bond, C488G substi- |
| | | | tution, and two poly charged |
| | | | amino acid residues added to |
| | | | the N-terminus and three |
| | | | poly charged amino acid res- |
| | | | idues added to the C-terminus. |
| | | | Identical to Scaffold #8 but |
| | | | for inclusion of C to S |
| | | | substitution in the first |
| | | | inserted immune-epitope. |
| Scaf- | VKAW [symbol] SKVGGNYNYLYRLGSGSG | 112 | Corresponds to residues 433-511 |
| fold | QAGSTPCNGVEGFNCYFPLQSY [symbol] N | | of SARS-CoV-2 S protein, but |
| #41 | GVG [symbol] RVV | | with backbone region between |
| | | | two critical binding motifs |
| | | | replaced with GS linker, |
| | | | repulsory residue Y473 deleted, |
| | | | and I434K. |
| | | | Identical to Scaffold #3 but |
| | | | for I434K substitution. |
| Scaf- | VIKW [symbol] SKVGGNYNYLYRLGSGSG | 113 | Corresponds to residues 433-511 |
| fold | QAGSTPCNGVEGFNCYFPLQSY [symbol] N | | of SARS-CoV-2 S protein, but |
| #42 | GVG [symbol] RVV | | with backbone region between |
| | | | two critical binding motifs , |
| | | | replaced with GS linker |
| | | | repulsory residue Y473 deleted, |
| | | | and A435K. |
| | | | Identical to Scaffold #3 but |
| | | | for A435K substitution. |
| Scaf- | [symbol] SKVGGNYNYLYRLGSGSGQAGST | 114 | Corresponds to residues 438-507- |
| fold | PCNGVEGFNCYFPLQSY [symbol] NGVG [symbol] | | OF SARS-CoV-2 S protein, but |
| #43 | [symbol] | | with backbone region between |
| | | | two critical binding motifs |
| | | | replaced with GS linker and |
| | | | repulsory Y473 deleted. |
| | | | Truncated version of Scaffolds |
| | | | #3, #12, #41, and #42. |
| Scaf- | [symbol] SKVGGNYNYLYRCGSGSGQAGST | 115 | Corresponds to residues 438-507- |
| fold | PGNGVEGFNGYFCLQSY [symbol] NGVG [symbol] | | OF SARS-CoV-2 S protein, but |
| #44 | [symbol] | | with backbone region between |
| | | | two critical binding motifs |
| | | | replaced with GS linker, |
| | | | repulsory Y473 deleted, L455C |
| | | | and P491C substitutions to |
| | | | introduce a disulfide bond, |
| | | | and C480G and C488G |
| | | | substitutions. |
| Scaf- | [symbol] SKVGGNYNYLYRLFDGTEIYQAGS | 116 | Corresponds to residues 438-507- |
| fold | TPCNGVEGFNCYFPLQSY [symbol] NGVG [symbol] | | OF SARS-CoV-2 S protein, but |
| #45 | [symbol] | | with majority of backbone |
| | | | region between two critical |
| | | | binding motifs replaced with |
| | | | DGT. |
| Scaf- | [symbol] SKVGGNYNYLYRLFNANDEIYQAG | 117 | Corresponds to residues 438-507- |
| fold | STPCNGVEGFNCYFPLQSY [symbol] NGVG | | OF SARS-CoV-2 S protein, but |
| #46 | [symbol] | | with majority of backbone |
| | | | region between two critical |
| | | | binding motifs replaced with |
| | | | NAND. |
| Scaf- | [symbol] SKVGGNYNYLYRLFNAHDKIYQAG | 118 | Corresponds to residues 438-507- |
| fold | STPCNGVEGFNCYFPLQSY [symbol] NGVG | | OF SARS-CoV-2 S protein, but |
| #47 | [symbol] | | with majority of backbone |
| | | | region between two critical |
| | | | binding motifs replaced with |
| | | | NAHDK. |
| Scaf- | [symbol] SKVGGNYNYLYRLFNANDKIYQAG | 119 | Corresponds to residues 438-507- |
| fold | STPCNGVEGFNCYFPLQSY [symbol] NGVG | | OF SARS-CoV-2 S protein, but |
| #48 | [symbol] | | with majority of backbone |
| | | | region between two critical |
| | | | binding motifs replaced with |
| | | | NANDK. |
| Scaf- | [symbol] SKVGGNYNYLYRLFDAHDKIYQAG | 120 | Corresponds to residues 438-507- |
| fold | STPCNGVEGFNCYFPLQSY [symbol] NGVG | | OF SARS-CoV-2 S protein, but |
| #49 | [symbol] | | with majority of backbone |
| | | | region between two critical |
| | | | binding motifs replaced with |
| | | | DAHDK. |
| Scaf- | [symbol] SKVGGNYNYLYRLFPKPEQAGST | 121 | Corresponds to residues 438-507- |
| fold | PCNGVEGFNCYFPLQSY [symbol] NGVG [symbol] | | OF SARS-CoV-2 S protein, but |
| #50 | [symbol] | | with majority of backbone |
| | | | region between two critical |
| | | | binding motifs replaced with |
| | | | PKPE and repulsory Y473 deleted. |
| Scaf- | [symbol] SKVGGNYNYLYRLFPGTEIYQAGS | 122 | Corresponds to residues 438-507- |
| fold | TPCNGVEGFNCYFPLQSY [symbol] NGVG [symbol] | | OF SARS-CoV-2 S protein, but |
| #51 | [symbol] | | with majority of backbone |
| | | | region between two critical |
| | | | binding motifs replaced with |
| | | | PGT. |
| Scaf- | [symbol] SKVGGNYNYLYRLFPATEIYQAGS | 123 | Corresponds to residues 438-507- |
| fold | TPCNGVEGFNCYFPLQSY [symbol] NGVG [symbol] | | OF SARS-CoV-2 S protein, but |
| #52 | [symbol] | | with majority of backbone |
| | | | region between two critical |
| | | | binding motifs replaced with |
| | | | PAT. |
| Scaf- | [symbol] SKVGGNYNYLYRLFPKPEIYQAGS | 124 | Corresponds to residues 438-507- |
| fold | TPCNGVEGFNCYFPLQSY [symbol] NGVG [symbol] | | OF SARS-CoV-2 S protein, but |
| #53 | [symbol] | | with majority of backbone |
| | | | region between two critical |
| | | | binding motifs replaced with |
| | | | PKP. |
| Scaf- | [symbol] SKVGGNYNYLYRLFPGTDIYQAGS | 125 | Corresponds to residues 438-507- |
| fold | TPCNGVEGFNCYFPLQSY [symbol] NGVG [symbol] | | OF SARS-CoV-2 S protein, but |
| #54 | [symbol] | | with majority of backbone |
| | | | region between two critical |
| | | | binding motifs replaced with |
| | | | PGTD. |
| Scaf- | [symbol] SKVGGNYNYLYRLFPAHDKIYQAG | 126 | Corresponds to residues 438-507- |
| fold | STPCNGVEGFNCYFPLQSY [symbol] NGVG | | OF SARS-CoV-2 S protein, but |
| #55 | [symbol] | | with majority of backbone |
| | | | region between two critical |
| | | | binding motifs replaced with |
| | | | PAHDK. |
| Scaf- | [symbol] VIAW [symbol] SKVGGNYNYLYRLFXXX | 127 | Corresponds to residues 433-511 |
| fold | XXXXXXXXXXXXXXXXXXXXXXXXXXXA [symbol] | | of SARS-CoV-2 S protein, but |
| #56 | [symbol] [symbol] RVV [symbol] | | with majority of backbone region |
| | [symbol] | | between two critical binding |
| | | | motifs and partial sequence of |
| | | | the second critical binding motif |
| | | | replaced with 5-30 AAs, C488G and |
| | | | P491S substitutions, and two poly |
| | | | charged amino acid residues added |
| | | | to the N-terminus and three poly |
| | | | charged amino acid residues added |
| | | | to the C-terminus. |

TABLE 3
Toxicity and antiviral activity of various CoV-2 scaffolds
Toxicity and antiviral activity of Ligandal compounds against SARS-CoV-2

| | | | | | | | | | | | Virus |
|---|---|---|---|---|---|---|---|---|---|---|---|
| | Percent Toxicity | | | | Virus Titer-CCID50/mL (Log10) | | | | | | Titer- |
| | Scaffold | Scaffold | Scaffold | Scaffold | Scaffold | Scaffold | Scaffold | Scaffold | | | CCID50/ |
| Concen- | #4 | #7 | #8 | #9 | #4 | #7 | #8 | #9 | Concen- | Percent | mL |
| tration | (SEQ ID | (SEQ ID | (SEQ ID | (SEQ ID | (SEQ ID | (SEQ ID | (SEQ ID | (SEQ ID | tration | Toxicity | (Log10) |
| (μg/ml) | NO: 75) | NO: 78) | NO: 79) | NO: 80) | NO: 75) | NO: 78) | NO: 79) | NO: 80) | (μg/ml) | M128533 | M128533 |
| 20 | 6.3% | 21.0% | 0.0% | 0.0% | 4.3 | 3.0 | 4.3 | 2.5 | 100 | 16.1% | <1.7 |
| 6.3 | 17.7% | 11.9% | 0.0% | 0.0% | 4.0 | 3.7 | 4.7 | 4.3 | 32 | 0.0% | <1.7 |
| 2 | 13.8% | 10.8% | 0.0% | 0.0% | 4.7 | 5.3 | 5.3 | 4.7 | 10 | 0.0% | 3.0 |
| 0.63 | 1.9% | 0.0% | 10.7% | 0.8% | 4.7 | 5.3 | 5.3 | 4.7 | 3.2 | 0.0% | 4.3 |
| 0.2 | 15.1% | 22.2% | 6.9% | 9.2% | 4.7 | 5.0 | 5.0 | 5.0 | 1 | 8.4% | 4.7 |
| 0.063 | 4.9% | 13.4% | 6.5% | 2.8% | 5.0 | 4.7 | 4.7 | 5.3 | 0.32 | 10.7% | 5.0 |
| 0.02 | 11.3% | 9.8% | 13.2% | 18.2% | 4.5 | 5.0 | 5.5 | 5.3 | 0.1 | 9.5% | 5.0 |
| 0.0063 | 0.0% | 7.8% | 12.3% | 0.0% | 4.7 | 4.7 | 5.0 | 5.0 | 0.032 | 10.0% | 4.7 |
| | | | | Virus | 4.5 | 4.7 | 4.5 | 5.0 | | | |
| | | | | Control | | | | | | | |

Percent toxicity determined by neutral red dye uptake on Caco-2 cells
50% cell culture infectious dose (CCID50) determined by endpoint dilution on Vero 76 cells

1.-43. (canceled)

44. A method of obtaining a scaffold that mimics the binding of the native protein from which the scaffold is derived, comprising:
producing a three-dimensional binding model of a first binding partner and a second binding partner,
determining the binding interface on each binding partner based on the binding model,
analyzing the binding interface to preserve the structure and/or conformation of each binding partner in its native, free, or bound state,
determining the critical binding residues based on thermodynamic calculation (ΔG), and
determining the amino acid sequence of the binding interface of each binding partner to obtain the scaffold.

45. The method of claim 44, wherein the three-dimensional binding is based on homology of either the first binding partner or the second binding partner to a protein of known sequence and/or structure.

46. The method of claim 45, further comprising designing scaffolds of various conformations or folding states to fit with the corresponding binding partner.

47. The method of claim 46, wherein the first binding partner and the second binding partner are SARS-CoV-2 spike protein and ACE2, respectively.

48. The method of claim 44, wherein the scaffold comprises a truncated peptide fragment from the binding interface of each of SARS-CoV-2 spike protein and ACE2 receptor, wherein the scaffold substantially maintains the structure, conformation, or binding affinity of the native SARS-CoV-2 spike protein or ACE2 receptor.

49. The method of claim 48, wherein the scaffold has a size of between 10 and 200 amino acid residues, from about 50 to about 100 amino acid residues, from about 55 to about 95 amino acid residues, from about 60 to about 90 amino acid residues, from about 65 to about 85 amino acid residues, from about 70 to about 80 amino acid residues.

50. The method of claim 49, wherein the scaffold has an amino acid sequence at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or 100% identical to the amino acid sequence of residues 433-511 of SEQ ID NO: 2, or to the amino acid sequence of residues 19-84 of SEQ ID NO: 140.

51. The method of claim 50, wherein the scaffold comprises a truncated peptide fragment from the binding interface of SARS-CoV-2 spike protein and maintains the β sheet structure, or comprises a truncated peptide fragment from the binding interface of ACE2 and maintains the α-helix structure.

52. The method of claim 51, wherein the scaffold comprises one or more modifications including insertions, deletions, or substitutions, provided that the one or more modifications do not substantially decrease the binding affinity of the scaffold to its binding partner.

53. The method of claim 52, wherein the scaffold further comprises one or more immuno-epitopes.

54. The method of claim 53, wherein the immuno-epitope is a T cell epitope or a B cell epitope.

55. The method of claim 54, wherein the immuno-epitope is selected from the group consisting of SEQ ID NOs: 7-64 and 67-71.

56. The method of claim 55, wherein the scaffold further comprises one or more conjugatable domains, and is attached to a nanoparticle, a chip, another substrate, another peptide, or another therapeutic agent via the conjugatable domain.

57. A multi-valent scaffold comprising two or more scaffolds, wherein the two or more scaffolds further comprise one or more conjugatable domains, and are attached to a nanoparticle, a chip, another substrate, another peptide, or another therapeutic agent via the conjugatable domain.

58. A composition comprising:
(a) one or more scaffolds comprising a truncated peptide fragment from the binding interface of each of SARS-CoV-2 spike protein and ACE2 receptor, wherein the one or more scaffolds substantially maintain the structure, conformation, or binding affinity of the native SARS-CoV-2 spike protein or ACE2 receptor;
(b) one or more multi-valent scaffolds comprising two or more scaffolds, wherein the two or more scaffolds comprise a truncated peptide fragment from the binding interface of each of SARS-CoV-2 spike protein and ACE2 receptor, wherein the two or more scaffolds substantially maintain the structure, conformation, or binding affinity of the native SARS-CoV-2 spike protein or ACE2 receptor;
(c) one or more fusion proteins comprising (a) and an immune-response eliciting domain; and
(d) one or more conjugates comprising (a) which are conjugated to another peptide, or another therapeutic agent.

59. The composition of claim 58, further comprising one or more pharmaceutically acceptable carriers, excipients, or diluents; wherein the composition is formulated into an injectable, inhalable, oral, nasal, topical, transdermal, uterine, or rectal dosage form, and is administered to a subject by a parenteral, oral, pulmonary, buccal, nasal, transdermal, rectal, or ocular route.

60. The composition of claim 59, wherein the composition is a vaccine composition.

61. A method of treating or preventing SAR-CoV-2 infection or blocking SAR-CoV-2 virus entry in a subject comprising administering to the subject a therapeutically effective amount of the composition of claim 59.

62. A method of targeted delivery of one or more therapeutic agents comprising conjugating the one or more therapeutic agents to the one or more scaffolds of (a) in claim 58 or to the multi-valent scaffold of (b) in claim 58 and delivering the conjugate to a subject in need thereof.

63. A method of targeted delivery of one or more therapeutic agents comprising (i) conjugating the one or more therapeutic agents to the one or more fusion proteins of (c) in claim 58, and delivering the conjugate of (i) or the conjugate of (d) of claim 58 to a subject in need thereof.
